SEQUENCE LISTING (18 sequences)

SEQ ID NO: 1 - protein, 6 aa, Artificial Sequence
  Description of Artificial Sequence Synthetic peptide
  MOD_RES (2)..(2): Phosphorylated Tyr
PYLKTK

SEQ ID NO: 2 - protein, 6 aa, Artificial Sequence
  Description of Artificial Sequence Synthetic peptide
  MOD_RES (1)..(1): Phosphorylated Tyr
YLPQTV

SEQ ID NO: 3 - DNA, 15 bp, Artificial Sequence
  Description of Artificial Sequence Synthetic oligonucleotide
  modified_base (1)..(1): Phosphorothioated c
  modified_base (2)..(2): Phosphorothioated a
  modified_base (3)..(3): Phosphorothioated t
  modified_base (12)..(12): Phosphorothioated a
  modified_base (13)..(13): Phosphorothioated a
  modified_base (14)..(14): Phosphorothioated t
catttcccgttaatc

SEQ ID NO: 4 - protein, 12 aa, Artificial Sequence
  Description of Artificial Sequence Synthetic peptide
SAVDRRAPAGRR

SEQ ID NO: 5 - protein, 14 aa, Artificial Sequence
  Description of Artificial Sequence Synthetic peptide
CMVDGARGPQILHR

SEQ ID NO: 6 - protein, 17 aa, Artificial Sequence
  Description of Artificial Sequence Synthetic peptide
LQEEDAGEYGCMVDGAR

SEQ ID NO: 7 - protein, 6 aa, Artificial Sequence
  Description of Artificial Sequence Synthetic peptide
LQEEDA

SEQ ID NO: 8 - protein, 6 aa, Artificial Sequence
  Description of Artificial Sequence Synthetic peptide
EDAGEY

SEQ ID NO: 9 - protein, 6 aa, Artificial Sequence
  Description of Artificial Sequence Synthetic peptide
GEYGCM

SEQ ID NO: 10 - protein, 12 aa, Artificial Sequence
  Description of Artificial Sequence Synthetic peptide
LQEEDAGEYGCM

SEQ ID NO: 11 - protein, 17 aa, Artificial Sequence
  Description of Artificial Sequence Synthetic peptide
LQVTDSGLYRCVIYHPP

SEQ ID NO: 12 - protein, 9 aa, Artificial Sequence
  Description of Artificial Sequence Synthetic peptide
GFLSKSLVF

SEQ ID NO: 13 - protein, 31 aa, Artificial Sequence
  Description of Artificial Sequence Synthetic polypeptide
  MOD_RES (23)..(23): Methionine sulfoxide
GFLSKSLVFPYLDDFQKKWQEEMELYRQKVE

SEQ ID NO: 14 - protein, 31 aa, Artificial Sequence
  Description of Artificial Sequence Synthetic polypeptide
  MOD_RES (15)..(15): Methionine sulfoxide
GFLSKSLVFPLGEEMRDRARAHVDALRTHLA

SEQ ID NO: 15 - protein, 7 aa, Artificial Sequence
  Description of Artificial Sequence Synthetic peptide
LSKSLVF

SEQ ID NO: 16 - protein, 7 aa, Artificial Sequence
  Description of Artificial Sequence Synthetic peptide
RGFFRGG

SEQ ID NO: 17 - protein, 17 aa, Artificial Sequence
  Description of Artificial Sequence Synthetic peptide
FPGVPLDTHDVFQYREK

SEQ ID NO: 18 - protein, 8 aa, Artificial Sequence
  Description of Artificial Sequence Synthetic peptide
VMDGYPMP

(19) United States
(12) Patent Application Publication
(10) Pub. No.: US 2023/0398146 A1
(43) Pub. Date: Dec. 14, 2023

(54) IMMUNE MODULATION OF MYELOID DERIVED SUPPRESSIVE CELL FUNCTION FOR CANCER TREATMENT

(71) Applicant: SURGE Therapeutics, Inc., Cambridge, MA (US)

(72) Inventor: Michael Solomon Goldberg, Brookline, MA (US)

(21) Appl. No.: 18/021,634

(22) PCT Filed: Aug. 17, 2021
(86) PCT No.: PCT/US2021/046392

§ 371 Date: Feb. 16, 2023

Related U.S. Application Data
(60) Provisional application No. 63/066,806, filed on Aug. 17, 2020.
(60) Provisional application No. 63/066,807, filed on Aug. 17, 2020.

(51) Int. Cl. A61K 35/17; A61K 31/728; A61K 31/722; A61K 47/69; A61P 35/00

(52) U.S. Cl. CPC A61K 35/17; A61K 31/728; A61K 31/722; A61K 47/6903; A61P 35/00

(57) ABSTRACT

The present disclosure provides technologies related to compositions each comprising a biomaterial preparation and a modulator of myeloid-derived suppressive cell function (e.g., a modulator of neutrophil function) as well as uses thereof for cancer treatment.

### CROSS REFERENCE TO RELATED APPLICATIONS

This application claims the benefit of U.S. Provisional Application No. 63/066,806 filed Aug. 17, 2020 and U.S. Provisional Application No. 63/066,807 filed Aug. 17, 2020, the contents of which are hereby incorporated herein in their entirety.

### BACKGROUND

Systemic administration of medication, nutrition, or other substances into the circulatory system affects the entire body. Systemic routes of administration include enteral (e.g., oral dosage resulting in absorption of the drug through the gastrointestinal tract) and parenteral (e.g., intravenous, intramuscular, and subcutaneous injections) administration. Administration of immunotherapeutics typically relies on these systemic administration routes, which can lead to unwanted side effects. In some instances, certain promising therapeutics are extremely difficult to develop due to associated toxicities and the limitations of current administration methods and systems.

Surgery is often the first-line of treatment for solid tumor cancers and is generally used in combination with systemic administration of anti-cancer therapy. However, surgery-induced immunosuppression has been implicated in the development of post-operative septic complications and tumor metastasis due to changes in a variety of metabolic and endocrine responses, ultimately resulting in the death of many patients (Hiller, J. G. et al. Nature Reviews Clinical Oncology, 2018, 15, 205-218).

### SUMMARY

Systemic administration of immunotherapies can result in adverse side effects, e.g., inducing toxicities that are undesirable for non-cancerous cells and/or tissues such as non-tumor-specific immune cells, and/or requiring high doses in order to achieve sufficient concentration at a target site to induce a therapeutic response; and surgical resection of tumors can result in immunosuppression. Surgery can also induce cellular stress, which may involve, for example, activation of one or more physiological responses that promote wound healing after injury. Such responses include, e.g., activation of neural, inflammatory, and/or pro-angiogenic signaling pathways, which can also promote the growth and/or metastatic spread of cancer. Inflammatory changes that may occur at a surgical site following tumor resection can include, e.g., recruitment of immune and/or inflammatory cell type(s) and/or release of humoral factor(s). Such changes in immune responses that may occur at a surgical site following tumor resection might promote or facilitate activation of dormant micrometastases and/or propagation of residual cancer cells, thus increasing the risk of cancer recurrence.

The present inventor has previously described various systems involving an immunomodulatory biomaterial independent of an immunomodulatory payload (see, for example, PCT/US20/31169, filed May 1, 2020 and now published as WO 2020/223698) or a combination of a biomaterial and an immunomodulatory payload (see, for example WO 2018/045058 or WO 2019/183216) that can be remarkably useful, among other things, when administered to subjects who have undergone or are undergoing tumor resection. Attributes of this system addressed the source of one or more problems associated with certain prior technologies including, for example, certain conventional approaches to cancer treatment. For example, this system could reduce and/or avoid certain adverse events (e.g., skin rashes, hepatitis, diarrhea, colitis, hypophysitis, thyroiditis, and adrenal insufficiency) that can be associated with systemic administration of immunotherapeutic agents. Among other things, this system could reduce or eliminate exposure of non-tumor-specific immune cells to systemically-administered immunotherapeutic drug(s) and/or to high doses of such drug(s) that are often required in order for systemic administration to achieve sufficient concentration in the tumor to induce a desired response; among other things, the system could provide local immunomodulation (e.g., local agonism of innate immunity) following tumor resection, which, among other things, can improve efficacy by concentrating the immunomodulatory effect where it is needed. Additionally or alternatively, such systems that provide local immunomodulation (e.g., agonism of innate immunity) following resection can, among other things, break local immune tolerance toward cancer and allow for development of systemic antitumor immunity, which can, for example, in some embodiments, lead to eradiation of disseminated disease.

The present disclosure provides a further surprising insight that local modulation of recruitment, survival, and/or immune effector function of immune cells following resection can be particularly useful and/or may provide particular beneficial effects, e.g., as described herein.

In certain aspects, without wishing to be bound by a particular theory, the present disclosure observes that inflammatory changes that occur at a surgical tumor resection can induce recruitment of numerous immune and/or inflammatory cell types and/or the release of humoral factors, thus promoting tumor capture and growth; moreover, recruited immune cells (e.g., MDSCs, neutrophils and/or macrophages) can secrete factors (e.g., VEGF and matrix metalloproteinases (MMPs)) that are known to promote growth and/or dissemination of cancer. See, e.g., Hiller et al. “Perioperative events influence cancer recurrence risk after surgery” Nature Reviews: Clinical Oncology (2018) 15: 205-218; and Tohme et al. “Surgery for Cancer: A Trigger for Metastases” Cancer Research (2017) 77: 1548-1552; the contents of which are incorporated herein in their entirety by reference for the purposes described herein. Further, in certain aspects, without wishing to be bound by a particular theory, the present disclosure observes that recruited neutrophils may react to injured tissues around a tumor resection site, for example, by forming neutrophil extracellular traps that facilitate entrapment and accumulation of circulating tumor cells; moreover, such web-like DNA neutrophil extracellular traps may contain a variety of molecules (e.g., proinflammatory molecules) that are useful for capture of tumor cells and/or augmented growth of metastases in surgically manipulated sites. See id.

The present disclosure, among other things, provides an insight that intraoperative modulation of neutrophil immune effector function(s) at a tumor resection site may be particularly useful and/or effective for cancer treatment. In some embodiments, such modulation may be useful and/or effective to reduce tumor relapse and/or regrowth. In some embodiments, such modulation may be useful and/or effective to reduce tumor metastasis. Indeed, among other things, the present disclosure teaches that intraoperative administration of a combination of a biomaterial (e.g., polymeric biomaterial, which in some embodiments may comprise a poloxamer) and a modulator of myeloid-derived suppressor cells (MDSCs) and, more particularly a combination of a biomaterial (e.g., polymeric biomaterial, which in some embodiments, may comprise a poloxamer) and a modulator of neutrophils as described herein, at a tumor resection site can provide beneficial therapeutic effects (e.g., ones as described herein). In some embodiments, such modulators of MDSCs and more particularly neutrophils that are useful for technologies described herein can inhibit recruitment and/or survival of such immune cells. Additionally or alternatively, in some embodiments such modulators of MDSCs and more particularly neutrophils that are useful for technologies described herein can modulate effector function, e.g., in some embodiments inhibit production of certain pro-tumorigenic factors and/or in some embodiments induce production of certain anti-tumorigenic factors.

In some aspects, provided are methods comprising intraoperatively administering at a target site (e.g., at or near a tumor resection site) of a subject suffering from cancer, a composition comprising a biomaterial (e.g., polymeric biomaterial) and a modulator of myeloid-derived suppressive cells (e.g., MDSCs, neutrophils, macrophages, monocytes, etc.). In some embodiments, a biomaterial (e.g., a polymeric biomaterial) may comprise one or more polymers, at least one of which is or comprises a poloxamer.

In some embodiments, the present disclosure provides compositions that can localize delivery of one or more modulators of myeloid-derived suppressive cells such as modulators of MDSCs and/or more particularly modulators of neutrophils to a target site (e.g., at or near a site at which a tumor has been removed and/or cancer cells have been treated or killed, e.g., by chemotherapy or radiation) and thereby concentrate the action of such modulators to a target site in need thereof. Such compositions can be particularly useful for treating cancer. In particular, compositions described herein may deliver one or more therapeutic agents that act on (e.g., modulate) one or more attributes of MDSCs and/or neutrophils such as recruitment, survival, and/or immune effector function of neutrophils, e.g., following a tumor resection, for the treatment of cancer, such as, for example, by preventing (e.g., delaying onset of, reducing extent of) tumor recurrence and/or metastasis, in some embodiments while minimizing adverse side effects and/or systemic exposure.

One aspect provided herein relates to a method comprising a step of intraoperative administration at a tumor resection site of a subject suffering from cancer: a combination of a biomaterial preparation and a modulator of myeloid-derived suppressive cell function. In particular embodiments, such a modulator of myeloid-derived suppressive cell function is or comprises a modulator of neutrophil function. In some embodiments, such a modulator of neutrophil function is or comprises an agent that (i) inhibits neutrophil survival and/or proliferation, and/or (ii) modulates neutrophil-associated effector function.

In certain embodiments, compositions described herein to be administered may deliver one or more agents that are characterized by their ability to modulate production and/or secretion of one or more immunomodulatory molecules produced by neutrophils. In certain embodiments, compositions described herein to be administered may deliver one or more agents that are characterized by their ability to modulate production and/or secretion of one or more immunomodulatory cytokines and/or chemokines, e.g., in some embodiments produced by neutrophils. In certain embodiments, such a modulator of neutrophil function is characterized in that it has the ability to inhibit production and/or secretion of one or more immunosuppressive cytokines and/or chemokines, e.g., in some embodiments produced by neutrophils. In certain embodiments, such a modulator of neutrophil function is characterized in that it has the ability to stimulate production and/or secretion of one or more immunostimulatory cytokines and/or chemokines, e.g., in some embodiments produced by neutrophils.

In certain embodiments, a modulator of neutrophil function that is useful in accordance with the present disclosure is characterized in that it has the ability to modulate recruitment, survival, and/or proliferation of neutrophils to a target site (e.g., a tumor resection site). For example, in some embodiments, such a modulator is characterized by its ability to modulate production and/or secretion of one or more cytokines and/or chemokines produced by immune cells (including, e.g., neutrophils).

In certain embodiments, a modulator of neutrophil function that is useful in accordance with the present disclosure is characterized in that it has the ability to modulate neutrophil-associated effector function. For example, in some embodiments, such a modulator is characterized by its ability to inhibit modification of extracellular matrix by neutrophils at a target site (e.g., a tumor resection site) of a subject in need thereof. In certain embodiments, such a modulator is characterized by its ability to inhibit formation of neutrophil extracellular trap (NET) that promote localization of tumor associated cells (e.g., by NETosis).

In certain embodiments, a modulator of MDSC and/or neutrophil function that may be useful in accordance with the present disclosure is or comprises at least one of the following: cathepsin G inhibitors, elastase inhibitors, CD74 inhibitors, CD47 inhibitors, adenosine pathway (CD39, CD73, A2AR, A2BR) inhibitors, ADAR1 inhibitors, matrix metalloproteinase (MMP) inhibitors, protein arginine deiminases 4 (PAD4) inhibitors, tyrosine kinases inhibitors, inhibitors of apoptosis proteins (IAP) inhibitors, bruton tyrosine kinase (BTK) inhibitors, purinergic receptor P2X 7 (P2RX7) inhibitors, colony stimulating factor 1 receptor (CSF1R) inhibitors, phosphodiesterase-5 (PDE5) inhibitors, activators of specialized pro-resolving mediators (SPMs), TGFβR1 inhibitors, CC chemokine inhibitors (e.g., CCR inhibitors, CCL inhibitors), CXC chemokine inhibitors (e.g., CXCR inhibitors, CXCL inhibitors), metformin, TREM-1 and/or TREM-2 inhibitors, interleukin 34 (IL-34) signaling inhibitors, purinergic receptor P2X4 (P2RX4) inhibitors, interleukin 1α (IL-1α) signaling inhibitors, dopaminergic receptor inhibitors and/or antipsychotic agents, neutropenia causing agents, TAM family receptor tyrosine kinase signaling pathway inhibitors, leukocyte-associated immunoglobulin-like receptor 1 (LAIR-1) inhibitors, leukocyte immunoglobulin-like receptor (LILR)-associated signaling pathway modulators, c-Kit related signaling pathway inhibitors, MET related signaling pathway inhibitors, interleukin-4 receptor (IL-4R) signaling inhibitors, monoamine oxidase A (MAO-A) inhibitors, complement component C5a and/or C5a receptor inhibitors, corticosteroids, glutamate-gated chloride channel activator and/or P2RX4, P2RX7, and/or alpha7 nicotinic acetylcholine receptor (a7 nAChR) positive allosteric effectors, beta-adrenergic receptor antagonists, renin-angiotensin system inhibitors, angiopoietin signaling modulators, or any combinations thereof.

In certain embodiments, a biomaterial preparation included in a composition described herein comprises one or more polymers. In certain embodiments, such a biomaterial preparation is temperature-responsive. For example, in certain embodiments, a temperature-responsive biomaterial preparations may be characterized by a critical gelation temperature (CGT) of 18-39° C. or 20-39° C. In certain embodiments, a temperature-responsive biomaterial preparation comprises a poloxamer (e.g., ones described herein). In certain embodiments, a temperature-responsive biomaterial preparation comprises a poloxamer (e.g., ones described herein) at a concentration of 12.5% (w/w) or below (e.g., 11% (w/w), 10.5% (w/w), 10% (w/w), 9% (w/w), 8% (w/w), 7% (w/w), 6% (w/w), 5% (w/w), 4% (w/w), or lower). In some embodiments, a poloxamer is present in a temperature-responsive biomaterial preparation at a concentration of 4% (w/w) to 11% (w/w), or 4% (w/w) to 10.5% (w/w), or 4% (w/w) to 10% (w/w). In some embodiments, a poloxamer is present in a temperature-responsive biomaterial preparation at a concentration of 5% (w/w) to 11% (w/w), or 5% (w/w) to 10.5% (w/w), or 5% (w/w) to 10% (w/w). In some embodiments, a poloxamer is present in a temperature-responsive biomaterial preparation at a concentration of 6% (w/w) to 11% (w/w), or 6% (w/w) to 10.5% (w/w), or 6% (w/w) to 10% (w/w). In some embodiments, a poloxamer that is useful in accordance with the present disclosure is or comprises poloxamer 407.

In certain embodiments, a temperature-responsive biomaterial preparation comprises a poloxamer (e.g., ones described herein) and at least one second polymer component that is not a poloxamer (e.g., ones described herein). In certain embodiments, such a second polymer component is or comprises a carbohydrate polymer. Examples of such a carbohydrate polymer may include but are not limited to hyaluronic acid, chitosan (including, e.g., a modified chitosan), and combinations thereof. In certain embodiments, at least one second polymer component (e.g., at least one carbohydrate polymer) may be present in a temperature-responsive biomaterial preparation at a concentration of below about 5% (w/w). In some embodiments, at least one second polymer (e.g., at least one carbohydrate polymer) may be present in a temperature-responsive biomaterial preparation at a concertation of 0.5% (w/w) to 10% (w/w), or 0.50% (w/w) to 5% (w/w), or 1% (w/w) to 10% (w/w), or 1% (w/w) to 5% (w/w), or 2% to 10% (w/w).

In certain embodiments where a second polymer component is or comprises hyaluronic acid, such hyaluronic acid can have an average molecular weight of about 50 kDa to about 2 MDa. In some embodiments, such hyaluronic acid may have an average molecular weight of 100 kDa to 500 kDa. In some embodiments, such hyaluronic acid may have an average molecular weight of 125 kDa to 375 kDa. In some embodiments, such hyaluronic acid may have an average molecular weight of 100 kDa to 400 kDa. In some embodiments, such hyaluronic acid may have an average molecular weight of 500 kDa to 1.5 MDa. In some embodiments, molecular weight of hyaluronic acid is characterized by weight average molecular weight. In some embodiments, molecular weight of hyaluronic acid is characterized by viscosity average molecular weight, which in some embodiments can be determined by converting intrinsic viscosity of hyaluronic acid to average molecular weight, for example, using the Mark-Houwink Equation. In some embodiments, molecular weight of hyaluronic acid can be measured by Size Exclusion Chromatography-Multiple Angle Laser Light Scattering (SEC-MALLS).

In some embodiments, number average molecular weight (Mn), weight average molecular weight (Mw), and/or dispersity (as characterized by polydispersity index) can be determined using SEC-MALLS.

In certain embodiments where a second polymer component is or comprises a chitosan or a modified chitosan, carboxymethyl chitosan may be used.

In certain embodiments, a biomaterial preparation has a storage modulus of about 100 Pa to about 50,000 Pa. In certain embodiments, a biomaterial preparation that is useful in accordance with the present disclosure is administered in a polymer network state. In some embodiments, a biomaterial preparation in a polymer network state is a hydrogel. In some embodiments, a biomaterial preparation in a polymer network state is a viscous solution or colloid. In certain embodiments, a biomaterial preparation that is useful in accordance with the present disclosure is administered in a precursor state such that the precursor state transitions to a polymer network state upon the administration at the tumor resection site.

In certain embodiments, a biomaterial preparation is biodegradable in vivo. In certain embodiments, a biomaterial preparation comprises at least one polymer component that is biodegradable in vivo. In certain embodiments, such a biomaterial preparation is characterized in that, when tested in vivo by administering the biomaterial preparation at a mammary fat pad of a mouse subject, less than or equal to 10% of the biomaterial (e.g., polymeric biomaterial) remains in vivo 4 months after the administration.

In certain embodiments, compositions described herein comprise a biomaterial preparation that forms a matrix or depot and a modulator of myeloid-derived suppressive cell function that is within the biomaterial preparation. In certain embodiments, a modulator of myeloid-derived suppressive cell function (e.g., a modulator of neutrophil function) is released from a biomaterial preparation after administration at a target site (e.g., a tumor resection site) by diffusion. For example, in certain embodiments, a polymer network state of a biomaterial preparation may be characterized in that, when tested in vitro by placing a composition comprising a biomaterial and a modulator of myeloid-derived suppressive cell function in PBS (pH 7.4), less than 100% of the modulator of myeloid-derived suppressive cell function is released within 3 hours from the biomaterial preparation. In certain embodiments, a polymer network state of a biomaterial preparation is characterized in that, when tested in vitro by placing a composition comprising a biomaterial and a modulator of myeloid-derived suppressive cell function in PBS (pH 7.4), at least 40% of the modulator of myeloid-derived suppressive cell function is released within 12 hours from the biomaterial preparation. In certain embodiments, a polymer network state of a biomaterial preparation is characterized in that, when tested in vivo by administering a composition comprising a biomaterial and a modulator of myeloid-derived suppressive cell function at a mammary fat pad of a mouse subject, less than or equal to 50% of the modulator of myeloid-derived suppressive cell function is released in vivo 8 hours after the administration. In certain embodiments, a polymer network state of a biomaterial preparation is characterized in that it extends release of a modulator of myeloid-derived suppressive cell function that is present in the biomaterial preparation so that, when assessed at 24 hours after administration, more modulator of myeloid-derived suppressive cell function is present at a target site (e.g., a tumor resection site than is observed when the modulator of myeloid-derived suppressive cell function is administered in solution.

In certain embodiments, compositions as described herein are monotherapeutic compositions in which a single modulator of myeloid-derived suppressive cell function is present in the absence of any other therapeutic agents. In some embodiments, compositions described herein may further comprise an additional therapeutic agent, which in some embodiments may be or comprise an immunomodulatory payload. Examples of such an additional immunomodulatory payload include but are not limited to modulators of innate immunity, modulators of myeloid cell function, modulators of adaptive immunity, modulators of inflammation, and/or combinations thereof.

In certain embodiments, a composition described herein is administered within 2 cm of a tumor resection site. In certain embodiments, a composition described herein is delivered to a tumor resection site that is characterized by the absence of gross residual tumor antigen.

In some embodiments, administration may be performed by implantation. For example, in some embodiments, a composition comprising a biomaterial preparation in a polymer network state (e.g., a hydrogel) may be administered by implantation.

In some embodiments, administration may be performed by injection. In some embodiments, injection may be performed by a robotic arm. For example, in some embodiments, a composition comprising a biomaterial preparation in a precursor state (e.g., a liquid state or an injectable state) is administered by injection, wherein the precursor state transitions to a polymer network state (e.g., a more viscous solution or colloid state or a hydrogel) upon the administration.

In some embodiments, administration may be performed concurrently with or subsequent to laparoscopy. In some embodiments, administration may be performed concurrently with or subsequent to a minimally invasive surgery (MIS), e.g., robot-assisted MIS, robotic surgery, and/or laparoscopic surgery, for tumor resection.

In certain embodiments, methods provided herein do not include administering adoptive transfer of T cells to a subject in need thereof. In certain embodiments, methods provided herein do not include administering a tumor antigen to a subject in need thereof. In certain embodiments, methods provided herein do not include administering a microparticle to a subject in need thereof.

Technologies provided herein are amenable to patients with cancer. In certain embodiments, such a cancer is metastatic. In certain embodiments, a cancer subject (e.g., with metastatic cancer) who has been administered a composition described herein may be monitored for indications of metastasis thereafter. For example, in some embodiments, a method provided herein may further comprise a step of monitoring at least one metastatic site in a subject in need thereof after administration of a provided composition.

These, and other aspects encompassed by the present disclosure, are described in more detail below and in the claims.

### BRIEF DESCRIPTION OF THE DRAWINGS

FIG. 1 is a graphical representation showing in vivo survival data of tumor resection animals administered with an exemplary composition comprising a polymeric biomaterial (e.g., comprising a combination of poloxamer, e.g., P407, with a low MW (e.g., −187 kDa) hyaluronic acid (HA)) and a modulator of myeloid-derived suppressive cell function such as, e.g., a Burton's tyrosine kinase (BTK) inhibitor (e.g., Zanubrutinib). Shown are results from a composition comprising 10% w/w poloxamer 407 and 3% w/w 187 kDa HA with a BTK inhibitor (e.g., Zanubrutinib, for example, in some embodiments at a dose of 1.25 mg/mouse), a control composition comprising 10% poloxamer 407 and 3% 187 kDa HA without a BTK inhibitor, and a control composition comprising 15% poloxamer 407. The x-axis indicates time post-tumor inoculation. Tumor resection was performed at Day 10 post-tumor inoculation, and an exemplary composition was administered following the tumor resection.

FIG. 2A-2B are graphical representations showing in vivo survival data of tumor resection animals administered with an exemplary compositions comprising a polymeric biomaterial (e.g., comprising a combination of poloxamer, e.g., P407, with a low MW (e.g., −187 kDa) hyaluronic acid (HA)) and a modulator of myeloid-derived suppressive cell function such as, e.g., a COX1 and/or COX2 inhibitor (e.g., Ketorolac). Shown are results from compositions comprising 10% w/w poloxamer 407 and 3% w/w 187 kDa HA with a COX1 and/or COX2 inhibitor (e.g., Ketorolac), control compositions comprising 10% poloxamer 407 and 3% 187 kDa HA without a COX1 and/or COX2 inhibitor, and control compositions comprising 15% poloxamer 407. FIG. 2A: Composition comprising 10% w/w poloxamer 407 and 3% w/w 187 kDa HA with a COX1 and/or COX2 inhibitor (e.g., Ketorolac, for example, in some embodiments at a dose of 6 mg/mouse). FIG. 2B: Composition comprising 10% w/w poloxamer 407 and 3% w/w 187 kDa HA with a COX1 and/or COX2 inhibitor (e.g., Ketorolac, for example, in some embodiments at a dose of 9 mg/mouse). The x-axis indicates time post-tumor inoculation. Tumor resection was performed at Day 10 post-tumor inoculation, and an exemplary composition was administered following the tumor resection.

FIG. 3 is a graphical representation showing in vivo survival data of tumor resection animals administered with an exemplary composition comprising a polymeric biomaterial (e.g., comprising a combination of poloxamer, e.g., P407, with a high MW (e.g., −766 kDa) hyaluronic acid (HA)) and a modulator of myeloid-derived suppressive cell function such as, e.g., a COX1 and/or COX2 inhibitor (e.g., Ketorolac). Shown are results from a composition comprising 9% w/w poloxamer 407 and 2.2% w/w 766 kDa HA with a COX1 and/or COX2 inhibitor (e.g., Ketorolac, for example, in some embodiments at a dose of 1.2 mg/mouse), and a control composition comprising 9% poloxamer 407 and 2.2% 766 kDa HA without a COX1 and/or COX2 inhibitor. The x-axis indicates time post-tumor inoculation. Tumor resection was performed at Day 10 post-tumor inoculation, and an exemplary composition was administered following the tumor resection.

FIG. 4 is a graphical representation showing in vivo survival data of tumor resection animals administered with an exemplary composition comprising a polymeric biomaterial (e.g., comprising a combination of poloxamer, e.g., P407, with a low MW (e.g., −187 kDa) hyaluronic acid (HA)) and a modulator of myeloid-derived suppressive cell function such as, e.g., a specialized pro-resolving mediator (e.g., Resolvin D2 (RvD2)). Shown are results from a composition comprising 10% w/w poloxamer 407 and 3% w/w 187 kDa HA with a specialized pro-resolving mediator (e.g., Resolvin D2 (RvD2), for example, in some embodiments at a dose of 2.5 μg/mouse), a control composition comprising 10% poloxamer 407 and 3% 187 kDa HA without a specialized pro-resolving mediator, and a control composition comprising 15% poloxamer 407. The x-axis indicates time post-tumor inoculation. Tumor resection was performed at Day 10 post-tumor inoculation, and an exemplary composition was administered following the tumor resection.

FIG. 5 is a graphical representation showing in vivo survival data of tumor resection animals administered with an exemplary composition comprising a polymeric biomaterial (e.g., comprising a combination of poloxamer, e.g., P407, with a low MW (e.g., −187 kDa) hyaluronic acid (HA)) and a modulator of myeloid-derived suppressive cell function such as, e.g., a CXCR4/CXCL12 signaling inhibitor (e.g., Plerixafor). Shown are results from a composition comprising 10% w/w poloxamer 407 and 3% w/w 187 kDa HA a CXCR4/CXCL12 signaling inhibitor (e.g., Plerixafor, for example, in some embodiments at a dose of 1.25 mg/mouse), a control composition comprising 10% poloxamer 407 and 3% 187 kDa HA without a CXCR4/CXCL12 signaling inhibitor, and a control composition comprising 15% poloxamer 407. The x-axis indicates time post-tumor inoculation. Tumor resection was performed at Day 10 post-tumor inoculation, and an exemplary composition was administered following the tumor resection.

FIG. 6 is a graphical representation showing in vivo survival data of tumor resection animals administered with an exemplary composition comprising a polymeric biomaterial (e.g., comprising a combination of poloxamer, e.g., P407, with a low MW (e.g., ˜187 kDa) hyaluronic acid (HA)) and a modulator of myeloid-derived suppressive cell function such as, e.g., an A2A and/or A2B adenosine receptor inhibitor (e.g., AB928, aka etrumadenant). Shown are results from a composition comprising 10% w/w poloxamer 407 and 3% w/w 187 kDa HA with an A2A and/or A2B adenosine receptor inhibitor (e.g., AB928, for example, in some embodiments at a dose of 1.25 mg/mouse), a control composition comprising 10% poloxamer 407 and 3% 187 kDa HA without an A2A and/or A2B adenosine receptor inhibitor, and a control composition comprising 15% poloxamer 407. The x-axis indicates time post-tumor inoculation. Tumor resection was performed at Day 10 post-tumor inoculation, and an exemplary composition was administered following the tumor resection.

FIG. 7 is a graphical representation showing in vivo survival data of tumor resection animals administered with an exemplary composition comprising a polymeric biomaterial (e.g., comprising a combination of poloxamer, e.g., P407, with a high MW (e.g., ˜766 kDa) hyaluronic acid (HA)) and a modulator of myeloid-derived suppressive cell function such as, e.g., an angiotensin II receptor antagonist (e.g., Valsartan). Shown are results from a composition comprising 11% w/w poloxamer 407 and 1.8% w/w 766 kDa HA with an angiotensin II receptor antagonist (e.g., Valsartan, for example, in some embodiments at a dose of 1 mg/mouse), and a control composition comprising 11% poloxamer 407 and 1.8% 766 kDa HA without an angiotensin II receptor antagonist. The x-axis indicates time post-tumor inoculation. Tumor resection was performed at Day 10 post-tumor inoculation, and an exemplary composition was administered following the tumor resection.

### CERTAIN DEFINITIONS

It is noted that the concentrations of individual polymer components in biomaterial preparations described herein are each expressed in % (w/w) or wt %. As used herein, the concentration, % (w/w), of a polymer component in a biomaterial preparation is determined based on the mass or weight of the polymer component relative to the sum of (i) total mass or weight of all individual polymer components present in the biomaterial preparation and (ii) total mass or weight solvent used in the biomaterial preparation.

Activator of adaptive immune response: The term “activator of adaptive immune response” refers to an agent that activates (e.g., increases the activity of) an adaptive immune system (and/or one or more features of an adaptive immune system) in a subject (e.g., in a subject to whom it is administered and/or who is otherwise in need thereof), as compared to when the agent is absent. Such activation can restore or enhance antitumor function, for example, by neutralizing inhibitory immune checkpoints and/or by triggering co-stimulatory receptors, ultimately generating helper and/or effector T cell responses against immunogenic antigens expressed by cancer cells and producing memory B cell, and/or T cell populations. In certain embodiments, an activator of adaptive immune response involves modulation of an adaptive immune response and/or leukocyte trafficking. Examples of activators of adaptive immune response include, e.g., ones described in WO 2018/045058, the contents of which are incorporated herein by reference in their entirety for the purposes described herein.

Activator of innate immune response: The term “activator of innate immune response” refers to an agent that activates (e.g., increases the activity of) an innate immune system (and/or one or more features of an innate immune system) in a subject (e.g., in a subject to whom it is administered and/or who is otherwise in need thereof), as compared to when the agent is absent. Such activation can stimulate (e.g., can increase expression level and/or activity of) one or more agents that initiate an inflammatory response (e.g., an immunostimulatory inflammatory response) and/or help to induce adaptive immune responses, for example, leading to the development of antigen-specific acquired immunity. In some embodiments, activation of the innate immune system can lead to recruitment of relevant immune cells including, e.g., but not limited to neutrophils, basophils, eosinophils, natural killer cells, dendritic cells, monocytes, and macrophages, cytokine production, leukocyte proliferation and/or survival, as well as improved T cell priming, for example by augmenting presentation of antigens and/or expression level and/or activity of co-stimulatory molecules by antigen-presenting cells. Examples of activators of innate immune response include, e.g., ones described in WO 2018/045058, the contents of which are incorporated herein by reference in their entirety for the purposes described herein.

Administer: As used herein, the term “administer,” “administering,” or “administration” typically refers to the administration of a composition to a subject to achieve delivery of an agent or payload that is, or is included in, a composition to a target site or a site to be treated. Those of ordinary skill in the art will be aware of a variety of routes that may, in appropriate circumstances, be utilized for administration of different agents to a subject, for example a human. For example, while the terms “administer,” “administering,” or “administration” refer to implanting, absorbing, ingesting, injecting, inhaling, parenteral administration, or otherwise introducing a composition as described herein, in the context of administering a composition comprising a composition described herein, administering may refer to, in some embodiments, implanting, or in some embodiments, injecting.

Agent: As used herein, the term “agent”, may refer to a physical entity or phenomenon. In some embodiments, an agent may be characterized by a particular feature and/or effect. In some embodiments, an agent may be a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Agonist: Those skilled in the art will appreciate that the term “agonist” may be used to refer to an agent, condition, or event whose presence, level, degree, type, or form correlates with increased level and/or activity of another agent (i.e., the agonized agent) and/or an increase in or induction of one or more biological events. In general, an agonist may be or include an agent of various chemical class including, for example, small molecules, polypeptides, nucleic acids, carbohydrates, lipids, metals, inorganic crystals, and/or any other entity that shows the relevant activating activity. In some embodiments, an agonist may be direct (in which case it exerts its influence directly upon its target); in some embodiments, an agonist may be indirect (in which case it exerts its influence by other than binding to its target; e.g., by interacting with a regulator of the target, so that level or activity of the target is altered). A partial agonist can act as a competitive antagonist in the presence of a full agonist, as it competes with the full agonist to interact with its target and/or a regulator thereof, thereby producing (i) a decrease in one or more effects of another agent, and/or (ii) a decrease in one or more biological events, as compared to that observed with the full agonist alone.

Antagonist: Those skilled in the art will appreciate that the term “antagonist” may refer to an agent, condition, or event whose presence, level, degree, type, or form is associated with a decreased level and/or activity of another agent (i.e., the antagonized agent) and/or a decrease in or suppression of one or more biological events. In general, an antagonist may include an agent of various chemical class including, for example, small molecules, polypeptides, nucleic acids, carbohydrates, lipids, metals, and/or any other entity that shows the relevant inhibitory activity. In some embodiments, an antagonist may be a “direct antagonist” in that it binds directly to its target; in some embodiments, an antagonist may be an “indirect antagonist” in that it exerts its influence by means other than binding directly to its target; e.g., by interacting with a regulator of the target, so that the level or activity of the target is altered).

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof, single chain Fvs; polypeptide-Fc fusions; single domain antibodies, alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)2, Fab′, di-sdFv, single domain antibodies, trifunctional antibodies, diabodies, and minibodies. etc. In some embodiments, relevant formats may be or include: Adnectins®; Affibodies®; Affilins®; Anticalins®; Avimers®; BiTE®s; cameloid antibodies; Centyrins®; ankyrin repeat proteins or DARPINs®; dual-affinity re-targeting (DART) agents; Fynomers®; shark single domain antibodies such as IgNAR; immune mobilizing monoclonal T cell receptors against cancer (ImmTACs); KALBITOR®s; MicroProteins; Nanobodies® minibodies; masked antibodies (e.g., Probodies®); Small Modular ImmunoPharmaceuticals (“SMIPs™”); single chain or Tandem diabodies (TandAb®); TCR-like antibodies; Trans-bodies®; TrimerX®; VHHs. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Bioadhesive: The term “bioadhesive” refers to a biocompatible agent that can adhere to a target surface, e.g., a tissue surface. In some embodiments, a bioadhesive can adhere to a target surface, e.g., a tissue surface, and retain on the target surface, e.g., for a period of time. In some embodiments, a bioadhesive may be biodegradable. In some embodiments, a bioadhesive may be a natural agent, which may have been prepared or obtained, for example, by isolation or by synthesis; in some embodiments, a bioadhesive may be a non-natural agent, e.g., as may have been designed and/or manufactured by the hand of man (e.g., by processing, synthetic, and/or recombinant production, depending on the agent, as will be understood by those skilled in the art. In some particular embodiments, a bioadhesive may be or comprise a polymeric material, e.g., as may be comprised of or contain a plurality of monomers such as sugars. Certain exemplary bioadhesives include a variety of FDA-approved agents such as, for example, cyanoacrylates (Dermabond, 2-Octyl cyanoacrylate; Indermil, n-Butyl-2-cyanoacrylate; Histoacryl and Histoacryl Blue, n-Butyl-2-cyanoacrylate), albumin and glutaraldehyde (BioGlue™, bovine serum albumin and 10% glutaraldehyde), fibrin glue (Tisseel™, human pooled plasma fibrinogen and thrombin; Evicel™, human pooled plasma fibrinogen and thrombin; Vitagel™, autologous plasma fibrinogen and thrombin; Cryoseal™ system, autologous plasma fibrinogen and thrombin), gelatin and/or resorcinol crosslinked by formaldehyde and/or glutaraldehyde, polysaccharide-based adhesives (e.g., alginate, chitosan, collagen, dextran, and/or gelatin), PEG, acrylates, polyamines, or urethane variants (isocyanate-terminated prepolymer, and/or combinations thereof. Other examples of bioadhesives that are known in the art, e.g., as described in Mehdizadeh and Yang “Design Strategies and Applications of Tissue Bioadhesives” MacromolBiosci 13:271-288 (2013), can be used for the purposes of the methods described herein. In some embodiments, a bioadhesive can be a degradable bioadhesive. Examples of such a degradable bioadhesive include, but are not limited to fibrin glues, gelatin-resorcinol-formaldehyde/glutaraldehyde glues, poly(ethylene glycol) (PEG)-based hydrogel adhesives, polysaccharide adhesives, polypeptide adhesives, polymeric adhesives, biomimetic bioadhesives, and ones described in Bhagat and Becker “Degradable Adhesives for Surgery and Tissue Engineering” Biomacromolecules 18: 3009-3039 (2017).

Biocompatible: The term “biocompatible”, as used herein, refers to materials that do not cause significant harm to living tissue when placed in contact with such tissue, e.g., in vivo. Biocompatibility of a material can be gauged by the ability of such a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity, and/or immunogenicity. In certain embodiments, materials are “biocompatible” if they themselves are not toxic to cells in an in vivo environment of its intended use. In certain embodiments, materials are “biocompatible” if their addition to cells in vitro results in less than or equal to 20% cell death and/or their administration in vivo does not induce significantly severe inflammation that is clinically undesirable for purposes described herein or other such adverse effects. As will be understood by those skilled in the art that such significantly severe inflammation is distinguishable from mild, transient inflammation, which typically accompanies surgery or introduction of foreign objects into a living organism. Furthermore, one of skill in the art will appreciate, reading the present disclosure, that in some embodiments, biomaterial preparations described herein and/or individual polymer components thereof are biocompatible if extent of immunomodulation (e.g., innate immunity agonism) over a defined period of time is clinically beneficial and/or desirable, e.g., to provide antitumor immunity.

Biodegradable: As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. As will be understood by one of ordinary skill in the art, the term “biodegradable” refers to partial biodegradability in some embodiments and total biodegradability in some embodiments. In certain embodiments, components generated by breakdown of a biodegradable material are biocompatible and therefore do not induce significantly severe inflammation that is clinically undesirable for purposes described herein and/or other adverse effects in vivo. In some embodiments, biodegradable polymer materials break down into their component monomers. In some embodiments, biodegradable polymer materials may be biologically degraded, e.g., by enzymatic activity or cellular machinery, in some cases, for example, through exposure to a lysozyme (e.g., having relatively low pH), or by simple hydrolysis. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid)(PLA), poly(glycolic acid)(PGA), poly(lactic-co-glycolic acid)(PLGA), and copolymers with PEG, polyanhydrides, poly(ortho)esters, polyesters, polyurethanes, poly(butyric acid), poly(valeric acid), poly(caprolactone), poly(hydroxyalkanoates), poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose variants and polyhydroxyalkanoates, for example, polyhydroxybutyrate blends and copolymers thereof. Those of ordinary skill in the art will appreciate or be able to determine when such polymers are biocompatible and/or biodegradable variants thereof (e.g., related to a parent polymer by substantially identical structure that differs only in substitution or addition of particular chemical groups as is known in the art).

Biologic: The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities such as cells and tissues. Biologics may be isolated from a variety of natural sources (e.g., human, animal, microorganism) and/or may be produced by biotechnological methods and/or other technologies.

Biomaterialpreparation: The term “biomaterial preparation” refers to a biocompatible composition characterized in that it can be administered to a subject for a medical purpose (e.g., therapeutic, diagnostic) without eliciting an unacceptable (according to sound medical judgement) reaction. Component(s) in a biomaterial preparation can be obtained or derived from nature or synthesized. In some embodiments, a biomaterial preparation may be or comprise a polymeric biomaterial. For example, in some embodiments, a polymeric biomaterial may comprise at least one or a plurality of (e.g., at least two or more) polymer components. For example, in some embodiments, a biomaterial preparation described herein is a biomaterial of a single polymer component (e.g., hyaluronic acid). In some embodiments, a biomaterial preparation described herein is a polymeric biomaterial comprising a first polymer component and a second first polymer component, wherein the first polymer component is or comprises at least one poloxamer, and the second polymer component is or comprises a polymer that is not poloxamer. In some embodiments, a biomaterial preparation can be in a polymer network state. In some embodiments, a biomaterial preparation can be in an injectable format, e.g., in a precursor state (e.g., a viscous solution). For example, a biomaterial precursor can comprise its precursor components to be formed in situ (e.g., upon administration to a subject). In some embodiments, a biomaterial preparation can be a liquid. In some embodiments, a biomaterial preparation is a viscous solution. In some embodiments, a biomaterial preparation is a colloid. In some embodiments, a biomaterial preparation can be a solid. In some embodiments, a biomaterial preparation can be a crystal (e.g., an inorganic crystal). In some embodiments, a biomaterial is not a nucleic acid. In some embodiments, a biomaterial is not a polypeptide.

Cancer: The term “cancer” refers to a malignant neoplasm (Stedman's Medical Dictionary, 25th ed.; Hensyl ed.; Williams & Wilkins: Philadelphia, 1990). Of particular interest in the context of some embodiments of the present disclosure are cancers treated by cell killing and/or removal therapies (e.g., surgical resection and/or certain chemotherapeutic therapies such as cytotoxic therapies, etc.). In some embodiments, a cancer that is treated in accordance with the present disclosure is one that has been surgically resected (i.e., for which at least one tumor has been surgically resected). In some embodiments, a cancer that is treated in accordance with the present disclosure is one for which resection is standard of care. In some embodiments, a cancer that is treated in accordance with the present disclosure is one that has metastasized. In certain embodiments, exemplary cancers may include one or more of acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bile duct cancer; bladder cancer; bone cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cardiac tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ductal carcinoma in situ; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; multiple myeloma; heavy chain disease (e.g., alpha chain disease, gamma chain disease, mu chain disease); hemangioblastoma; histiocytosis; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); melanoma; midline tract carcinoma; multiple endocrine neoplasia syndrome; muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); nasopharynx cancer; neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); parathyroid cancer; papillary adenocarcinoma; penile cancer (e.g., Paget's disease of the penis and scrotum); pharyngeal cancer; pinealoma; pituitary cancer; pleuropulmonary blastoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; retinoblastoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; stomach cancer; small intestine cancer; sweat gland carcinoma; synovioma; testicular cancer (e.g., seminoma, testicular embryonal carcinoma); thymic cancer; thyroid cancer (e.g., papillary carcinoma of the thyroid, papillary thyroid carcinoma (PTC), medullary thyroid cancer); urethral cancer; uterine cancer; vaginal cancer; and vulvar cancer (e.g., Paget's disease of the vulva).

Carbohydrate polymer: The term “carbohydrate polymer” refers to a polymer that is or comprises one or more carbohydrates, e.g., having a carbohydrate backbone. For example, in some embodiments, a carbohydrate polymer refers to a polysaccharide or an oligosaccharide, or a polymer containing a plurality of monosaccharide units connected by covalent bonds. The monosaccharide units may all be identical, or, in some cases, there may be more than one type of monosaccharide unit present within the carbohydrate polymer. In certain embodiments, a carbohydrate polymer is naturally occurring. In certain embodiments, a carbohydrate polymer is synthetic (i.e., not naturally occurring). In some embodiments, a carbohydrate polymer may comprise a chemical modification. In some embodiments, a carbohydrate polymer is a linear polymer. In some embodiments, a carbohydrate polymer is a branched polymer.

Chemotherapeutic agent: The term “chemotherapeutic agent” refers to a therapeutic agent known to be of use in chemotherapy for cancer. For example, in some embodiments, a chemotherapeutic agent can inhibit the proliferation of rapidly growing cancer cells and/or kill cancer cells. Examples of such chemotherapeutic agents include, but are not limited to alkylating agents, anti-metabolites, topoisomerase inhibitors, and/or mitotic inhibitors.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Colloid: As used herein, the term “colloid” refers to a homogenous solution or suspension of particles (e.g., polymer particles) dispersed though a continuous medium (e.g., an aqueous buffer system). In some embodiments, a colloid is an emulsion. In some embodiments, a colloid is a sol. In some embodiments, a colloid is a gel.

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied. Those of ordinary skill in the art will also understand that when the term “comparable” is used in the context of comparison of two or more values, such values are comparable to one another such that the differences in values do not result in material differences in therapeutic outcomes, e.g., induction of anti-tumor immunity and/or incidence of tumor regrowth and/or metastasis. For example, in some embodiments, comparable release rates refer to values of such release rates within 15% over a period of 48 hours. In some embodiments, comparable release rates refer to values of such release rates within 20% over a period of 48 hours. In some embodiments, comparable release rates refer to values of such release rates within 15% over a period of 24 hours.

Critical gelation temperature: As used herein, the term “critical gelation temperature”, abbreviated as “CGT”, refers to a threshold temperature at or above which a precursor state of a biomaterial preparation (e.g., ones described herein) transitions to a polymer network state described herein (e.g., a hydrogel state). In some embodiments, a critical gelation temperature may correspond to a sol-gel transition temperature. In some embodiments, a critical gelation temperature may correspond to a lower critical solution temperature. See Taylor et al., “Thermoresponsive Gels” Gels (2017) 3:4, for general description of thermoresponsive gels, the contents of which are incorporated herein by reference for purposes described herein. As described in the present disclosure, certain embodiments of biomaterial preparations described herein are demonstrated to form a polymer network state when it is exposed to a temperature of about 35-40° C. One of ordinary skill in the art, reading the present disclosure, will understand that such biomaterial preparations do not necessarily have a CGT of about 35-40° C., but may rather have a CGT that is lower than 35-40° C. For example, in some embodiments, provided biomaterial preparations may have a CGT of about 20-28° C.

Crosslink: As used herein, the term “crosslink” refers to interaction and/or linkage between one entity and another entity to form a network. For example, in some embodiments, crosslinks present in polymer network may be or comprise intra-molecular crosslinks, inter-molecular crosslinks, or both. In some embodiments, crosslinks may comprise interactions and/or linkages between one polymer chain(s) and another polymer chain(s) to form a polymer network. In some embodiments, a crosslink may be achieved using one or more physical crosslinking approaches, including, e.g., one or more environmental triggers and/or physiochemical interactions. Examples of an environmental trigger include, but are not limited to pH, temperature, and/or ionic strength. Non-limiting examples of physiochemical interactions include hydrophobic interactions, charge interactions, hydrogen bonding interactions, stereocomplexation, and/or supramolecular chemistry. In some embodiments, a crosslink may be achieved using one or more covalent crosslinking approaches (e.g., where the linkage between two entities is or comprises a covalent bond) based on chemistry reactions, e.g., in some embodiments which may include reaction of an aldehyde and an amine to form a Schiff base, reaction of an aldehyde and hydrazide to form a hydrazine, and/or Michael reaction of an acrylate and either a primary amine or a thiol to form a secondary amine or a sulfide. Examples of such covalent crosslinking approaches include, but are not limited to small-molecule crosslinking and polymer-polymer crosslinking. Various methods for physical and covalent crosslinking of polymer chains are known in the art, for example, as described in Hoare and Kohane, “Hydrogels in drug delivery: Progress and challenges” Polymer (2008) 49:1993-2007, the entire content of which is incorporated herein by reference for the purposes disclosed herein.

Crosslinker: As used interchangeably herein, the term “crosslinker” or “crosslinking agent” refers to an agent that links one entity (e.g., one polymer chain) to another entity (e.g., another polymer chain). In some embodiments, linkage (i.e., the “crosslink”) between two entities is or comprises a covalent bond. In some embodiments, linkage between two entities is or comprises an ionic bond or interaction. In some embodiments, a crosslinker is a chemical crosslinker, which, e.g., in some embodiments may be or comprise a small molecule (e.g., dialdehydes or genipin) for inducing formation of a covalent bond between an aldehyde and an amino group. In some embodiments, a crosslinker comprises a photo-sensitive functional group. In some embodiments, a crosslinker comprises a pH-sensitive functional group. In some embodiments, a crosslinker comprises a thermal-sensitive functional group.

Effective amount: An “effective amount” is an amount sufficient to elicit a desired biological response, e.g., treating a condition from which a subject may be suffering. As will be appreciated by those of ordinary skill in this art, the effective amount of a composition or an agent included in the composition may vary depending on such factors as the desired biological endpoint, the physical, chemical, and/or biological characteristics (e.g., pharmacokinetics and/or degradation) of agents in the composition, the condition being treated, and the age and health of the subject. In some embodiments, an amount may be effective for therapeutic treatment; alternatively or additionally, in some embodiments, an amount may be effective for prophylactic treatment. For example, in treating cancer, an effective amount may prevent tumor regrowth, reduce the tumor burden, or stop the growth or spread of a tumor. Those skilled in the art will appreciate that an effective amount need not be contained in a single dosage form. Rather, administration of an effective amount may involve administration of a plurality of doses, potentially over time (e.g., according to a dosing regimen). For example, in some embodiments, an effective amount may be an amount administered in a dosing regimen that has been established, when administered to a relevant population, to achieve a particular result with statistical significance.

Hydrate: The term “hydrate”, as used herein, has its art-understood meaning and refers to an aggregate of a compound (which may, for example be a salt form of the compound) and one or more water molecules. Typically, the number of the water molecules contained in a hydrate of a compound is in a definite ratio to the number of the compound molecules in the hydrate. Therefore, a hydrate of a compound may be represented, for example, by the general formula R××H₂O, wherein R is the compound and x is a number greater than 0. A given compound may form more than one type of hydrate, including, e.g., monohydrates (x is 1), lower hydrates (x is a number greater than 0 and smaller than 1, e.g., hemihydrates (R×0.5 H₂O)), and polyhydrates (x is a number greater than 1, e.g., dihydrates (R×2 H₂O) and hexahydrates (R×6 H₂O)).

Hydrogel: The term “hydrogel” has its art-understood meaning and refers to a material formed from a network of polymer chains that are hydrophilic, sometimes found as a colloidal gel in which an aqueous phase is the dispersion medium. In some embodiments, hydrogels are highly absorbent (e.g., they can absorb and/or retain over 90% water) natural or synthetic polymeric networks. In some embodiments, hydrogels possess a degree of flexibility similar to natural tissue, for example due to their significant water content.

Immunotherapy: The term “immunotherapy” refers to a therapeutic agent that promotes the treatment of a disease by inducing, enhancing, or suppressing an immune response. Immunotherapies designed to elicit or amplify an immune response are classified as activation immunotherapies, while immunotherapies that reduce or suppress an immune response are classified as suppression immunotherapies. Immunotherapies are typically, but not always, biotherapeutic agents. Numerous immunotherapies are used to treat cancer. These include, but are not limited to, monoclonal antibodies, adoptive cell transfer, cytokines, chemokines, vaccines, nucleic acids, small molecule inhibitors, and small molecule agonists. For example, useful immunotherapies may include, but are not limited to, inducers of type I interferon, interferons, stimulator of interferon genes (STING) agonists, TLR7/8 agonists, IL-15 superagonists, COX inhibitors (e.g., COX-1 inhibitors and/or COX-2 inhibitors), anti-PD-1 antibodies, anti-CD137 antibodies, and anti-CTLA-4 antibodies. In some embodiments, certain biomaterial preparations provided herein are themselves immunomodulatory (e.g., sufficient to induce anti-tumor immunity) in the absence of immunotherapy and thus do not include administration of such immunotherapy as described herein.

Immunomodulatory payload: As used herein, the term “immunomodulatory payload” refers to a separate immunomodulatory agent (e.g., small molecules, polypeptides (including, e.g., cytokines), nucleic acids, etc.) that can be carried by or distributed in a biomaterial preparation such as ones as provided and/or utilized herein), wherein the immunomodulatory agent provides a therapeutic effect of modulating or altering (e.g., inducing, enhancing, or suppressing, etc.) one or more aspects of an immune response in a subject. Examples of an immunomodulatory payload include, but are not limited to activators of adaptive immune response, activators of innate immune response, inhibitors of a proinflammatory pathway, immunomodulatory cytokines, or immunomodulatory therapeutic agents as well as ones as described in WO 2018/045058 and WO 2019/183216, and any combinations thereof. The contents of the aforementioned patent applications are incorporated herein by reference for the purposes described herein. In some embodiments, an immunomodulatory payload is or comprises an innate immunity modulatory payload (e.g., an immunomodulatory payload that induces or stimulates innate immunity and/or one or more features of innate immunity). In some embodiments, an innate immunity modulatory payload is or comprises an activator of innate immune response. In some embodiments, an immunomodulatory payload is or comprises an adaptive immunity modulatory payload, e.g., an activator of adaptive immune response. In some embodiments, an immunomodulatory payload is or comprises an inhibitor of a proinflammatory pathway, e.g., an inhibitor of proinflammatory immune response mediated by a p38 mitogen-activated protein kinase (MAPK) pathway. In some embodiments, an immunomodulatory payload is or comprises an immunomodulatory cytokine. In some embodiments, an immunomodulatory payload is or comprises an immunomodulatory therapeutic agent. As will be understood by those skilled in the art, an immunomodulatory payload does not include components (e.g., precursor components) and/or by-products of a biomaterial preparation (e.g., as described and/or utilized herein) generated, e.g., by chemical, enzymatic, and/or biological reactions such as, e.g., degradation.

Implanting: The terms “implantable,” “implantation,” “implanting,” and “implant” refer to positioning a composition of interest at a specific location in a subject, such as within a tumor resection site or in a sentinel lymph node, and typically by general surgical methods.

Increased, Induced, or Reduced: As used herein, these terms or grammatically comparable comparative terms, indicate values that are relative to a comparable reference measurement. For example, an assessed value achieved in a subject may be “increased” relative to that obtained in the same subject under different conditions (e.g., prior to or after an event; or presence or absence of an event such as administration of a composition or preparation as described and/or utilized herein, or in a different, comparable subject (e.g., in a comparable subject that differs from the subject of interest in prior exposure to a condition, e.g., absence of administration of a composition or preparation as described and/or utilized herein.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Inhibit: The term “inhibit” or “inhibition” is not limited to only total inhibition. Thus, in some embodiments, partial inhibition or relative reduction is included within the scope of the term “inhibition.” For example, in the context of modulating level (e.g., expression and/or activity) of a target, the term, in some embodiments, refers to a reduction in the level (e.g., expression and/or activity) of a target to a level that is reproducibly and/or statistically significantly lower than an initial or other appropriate reference level, which may, for example, be a baseline level of a target. In some embodiments, the term refers to a reduction in the level (e.g., expression and/or activity) of a target to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of a target. In the context of risk and/or incidence of tumor recurrence and/or metastasis, the term, in some embodiments, refers to a reduction of the risk or incidence of tumor recurrence and/or metastasis to a level that is reproducibly and/or statistically significantly lower than an initial or other appropriate reference level, which may, for example, be a baseline level of risk or incidence of tumor recurrence and/or metastasis in the absence or prior to administration of a composition described herein. In some embodiments, the term refers to a reduction of the risk or incidence of tumor recurrence and/or metastasis to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of risk or incidence of tumor recurrence and/or metastasis in the absence or prior to administration of a composition described herein. In the context of modulation of an immune cell function (e.g., by inhibiting activity and/or expression of a target), the term, in some embodiments, refers to a reduction of the activity and/or expression of a target to a level that is reproducibly and/or statistically significantly lower than an initial or other appropriate reference level, which may, for example, be a baseline level of activity and/or expression of the target in the absence or prior to administration of a composition described herein.

Inhibitor: As used herein, the term “inhibitor” refers to an agent whose presence or level correlates with decreased level or activity of a target to be modulated. In some embodiments, an inhibitor may act directly (in which case it exerts its influence directly upon its target, for example by binding to the target); in some embodiments, an inhibitor may act indirectly (in which case it exerts its influence by interacting with and/or otherwise altering a regulator of a target, so that level and/or activity of the target is reduced). In some embodiments, an inhibitor is one whose presence or level correlates with a target level or activity that is reduced relative to a particular reference level or activity (e.g., that observed under appropriate reference conditions, such as presence of a known inhibitor, or absence of the inhibitor as disclosed herein, etc.). In some embodiments, an inhibitor may be a small molecule, a polynucleotide, an oligonucleotide, a polysaccharide, a polypeptide, a protein, an antibody, and/or a functional portion thereof.

Isomers: It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Metastasis: The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

Microparticle: As used herein, the term “microparticle” refers to a particle having a longest dimension (e.g., diameter) between 1 micrometer and 1000 micrometers (μm). In some embodiments, a microparticle may be characterized by a longest dimension (e.g., a diameter) of between 1 μm and 500 μm. In some embodiments, a microparticle may be characterized by a longest dimension (e.g., a diameter) of between 1 μm and 100 μm. In many embodiments, a population of microparticles is characterized by an average size (e.g., longest dimension) that is below about 1,000 μm, about 500 μm, about 100 μm, about 50 μm, about 40 μm, about 30 μm, about 20 μm, or about 10 m and often above about 1 μm. In many embodiments, a microparticle may be substantially spherical (e.g., so that its longest dimension may be its diameter.

Monosaccharide: As used herein, the term “monosaccharide” is given its ordinary meaning as used in the art and refers to a simple form of a sugar that consists of a single saccharide unit which cannot be further decomposed to smaller saccharide building blocks or moieties. Common examples of monosaccharides include, e.g., glucose (dextrose), fructose, galactose, mannose, ribose, etc. Monosaccharides can be classified according to the number of carbon atoms of the carbohydrate, for example, triose, having 3 carbon atoms such as glyceraldehyde and/or dihydroxyacetone; tetrose, having 4 carbon atoms such as erythrose, threose and/or erythrulose; pentose, having 5 carbon atoms such as arabinose, lyxose, ribose, xylose, ribulose and/or xylulose; hexose, having 6 carbon atoms such as allose, altrose, galactose, glucose, gulose, idose, mannose, talose, fructose, psicose, sorbose and/or tagatose; heptose, having 7 carbon atoms such as mannoheptulose, and/or sedoheptulose; octose, having 8 carbon atoms such as 2-keto-3-deoxy-manno-octonate; nonose, having 9 carbon atoms such as sialose; and decose, having 10 carbon atoms. The above monosaccharides encompass both D- and L-monosaccharides. Alternatively, a monosaccharide can be a monosaccharide variant, in which the saccharide unit comprises one or more substituents (e.g., deoxy, H substituents, heteroatom substituents (e.g., S, Cl, F, etc.), etc.) other than a hydroxyl. Such variants can be, but are not limited to, ethers, esters, amides, acids, phosphates and amines. Amine variants (i.e., amino sugars) include, for example, glucosamine, galactosamine, fructosamine and/or mannosamine. Amide variants include, for example, N-acetylated amine variants of saccharides (e.g., N-acetylglucosamine, and/or N-acetylgalactosamine).

Modulator: As used herein, the term “modulator” may be or comprise an entity whose presence or level in a system in which an activity of interest is observed correlates with a change in level and/or nature of that activity as compared with that observed under otherwise comparable conditions when the modulator is absent. In some embodiments, a modulator is an activator or agonist, in that an activity of interest is increased in its presence as compared with that observed under otherwise comparable conditions when the modulator is absent. In some embodiments, a modulator is an antagonist or inhibitor, in that an activity of interest is reduced in its presence as compared with otherwise comparable conditions when the modulator is absent. In some embodiments, a modulator interacts directly with a target entity whose activity is of interest. In some embodiments, a modulator interacts indirectly (e.g., interacts with one or more entities that interacts and/or are associated with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level. In some embodiments, a modulator may be a small molecule, a polynucleotide, an oligonucleotide, a polysaccharide, a polypeptide, a protein, an antibody, and/or a functional portion thereof.

Modulator of Neutrophil Function: As used interchangeably herein, the terms “modulator of neutrophils” and “modulator of neutrophil function” refer to a modulator of one or more biological functions and/or phenotypes of neutrophils. For example, in some embodiments, a modulator of neutrophil function can inhibit recruitment, survival, and/or proliferation of neutrophils. Additionally or alternatively, in some embodiments, a modulator of neutrophil function can modulate neutrophil-associated effector function, which may include but are not limited to, modulation of production and/or secretion of one or more immunomodulatory molecules (e.g., immunomodulatory cytokines and/or chemokines) and/or alter extracellular-matrix modifying capabilities of neutrophils. In some embodiments, a modulator of neutrophil function (e.g., ones described herein) may act on or target neutrophils only. In some embodiments, a modulator of neutrophil function (e.g., ones described herein) may act on neutrophils and at least one additional type of immune cells, e.g., other subsets of myeloid-derived suppressive cells (MDSCs), macrophages, and/or monocytes. One of ordinary skill in the art will appreciate that at least a subset of neutrophils may exhibit similar immune activities as one or more certain subsets of MDSCs and thus be considered as polymorphonuclear and/or granulocytic MDSCs (for example, as described in: Mehmeti-Ajradini et al., “Human G-MDSCs are neutrophils at distinct maturation stages promoting tumor growth in breast cancer” Life Science Alliance, Sep. 21, 2020; and Brandau et al., “A subset of mature neutrophils contains the strongest PMN-MDSC activity in blood and tissue of patients with head and neck cancer” The Journal of Immunology, May 1, 2020; the contents of each of which are incorporated herein by reference for purposes described herein).

Nanoparticle: As used herein, the term “nanoparticle” refers to a particle having a longest dimension (e.g., a diameter) of less than 1000 nanometers (nm). In some embodiments, a nanoparticle may be characterized by a longest dimension (e.g., a diameter) of less than 300 nm. In some embodiments, a nanoparticle may be characterized by a longest dimension (e.g., a diameter) of less than 100 nm. In many embodiments, a nanoparticle may be characterized by a longest dimension between about 1 nm and about 100 nm, or between about 1 nm and about 500 nm, or between about 1 nm and 1,000 nm. In many embodiments, a population of nanoparticles is characterized by an average size (e.g., longest dimension) that is below about 1,000 nm, about 500 nm, about 100 nm, about 50 nm, about 40 nm, about 30 nm, about 20 nm, or about 10 nm and often above about 1 nm. In many embodiments, a nanoparticle may be substantially spherical so that its longest dimension may be its diameter. In some embodiments, a nanoparticle has a diameter of less than 100 nm as defined by the National Institutes of Health.

Neoplasm and tumor: The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An example of a pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

Payload: In general, the term “payload”, as used herein, refers to an agent that may be incorporated into a biomaterial preparation described herein. In some embodiments, a payload may refer to a compound, molecule, or entity of any chemical class including, for example, a small molecule, a peptide, a polypeptide, a nucleic acid, a saccharide (e.g., a polysaccharide), a lipid, a metal, or a combination or complex thereof. In some embodiments, a payload may be or comprise a biological modifier, a detectable agent (e.g., a dye, a fluorophore, a radiolabel, etc.), a detecting agent, a nutrient, a therapeutic agent, a mineral, a growth factor, a cytokine, an antibody, a hormone, an extracellular matrix protein (such as collagen, vitronectin, fibrin, etc.), an extracellular matrix sugar, a chemoattractant, a polynucleotide (e.g., DNA, RNA, antisense molecule, plasmid, etc.), a microorganism (e.g., a virus), etc., or a combination thereof. In some embodiments, a payload is or comprises a therapeutic agent. Examples of a therapeutic agent include but are not limited to analgesics, antibiotics, antibodies, anticoagulants, antiemetics, cells, coagulants, cytokines, growth factors, hormones, immunomodulatory agents, polynucleotides (e.g., DNA, RNA, antisense molecules, plasmids, etc.), and combinations thereof. In some embodiments, a payload may be or comprise a cell or organism, or a fraction, extract, or component thereof. Alternatively or additionally, in some embodiments, a payload may be or comprise a natural product in that it is found in and/or is obtained from nature. Alternatively or additionally, in some embodiments, the term may be used to refer to one or more entities that is man-made in that it is designed, engineered, and/or produced through action of the hand of man and/or is not found in nature. In some embodiments, a payload may be or comprise an agent in isolated or pure form; in some embodiments, such an agent may be in crude form.

Pharmaceutically acceptable salt: The term “pharmaceutically acceptable salt” refers to those salts which are, within the scope of sound medical judgment, suitable for use in contact with the tissues of, for example, humans and/or animals without undue toxicity, irritation, allergic response, and the like and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, the contents of which are incorporated herein by reference for purposes described herein. Pharmaceutically acceptable salts that may be utilized in accordance with certain embodiments of the present disclosure may include, for example, those derived from suitable inorganic and organic acids and bases. Examples of pharmaceutically acceptable, non-toxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁-C₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Poloxamer: As used herein, the term “poloxamer” refers to a polymer preparation of or comprising one or more poloxamers. In some embodiments, poloxamers in a polymer preparation may be unconjugated or unmodified, for example, which are typically triblock copolymers comprising a hydrophobic chain of polyoxypropylene (polypropylene glycol, PPG) flanked by two hydrophilic chains of polyoxyethylene (polyethylene glycol, PEG). In some embodiments, a polymer preparation of or comprising one or more poloxamer may be unfiltered (e.g., such a polymer preparation may contain impurities and/or relatively low molecular weight polymeric molecules, as compared to a comparable polymer preparation that is filtered). Examples of poloxamers include are not limited to, Poloxamer 124 (P124, also known as Pluronic L44 NF), Poloxamer 188 (P188, also known as Pluronic F68NF), Poloxamer 237 (P237, also known as Pluronic F 87 NF), Poloxamer 338 (P338, also known as Pluronic F108 NF), Poloxamer 407 (P407, also known as Pluronic F127 NF), and combinations thereof.

Polymer: The term “polymer” is given its ordinary meaning as used in the art, i.e., a molecular structure comprising one or more repeat units (monomers), connected by covalent bonds. The repeat units may all be identical, or, in some cases, there may be more than one type of repeat unit present within the polymer (e.g., in a copolymer). In certain embodiments, a polymer is naturally occurring. In certain embodiments, a polymer is synthetic (i.e., not naturally occurring). In some embodiments, a polymer is a linear polymer. In some embodiments, a polymer is a branched polymer. In some embodiments, a polymer for use in accordance with the present disclosure is not a polypeptide. In some embodiments, a polymer for use in accordance with the present disclosure is not a nucleic acid.

Polymeric biomaterial: A “polymeric biomaterial”, as described herein, is a material that is or comprises at least one polymer or at least one polymeric moiety and is biocompatible. In many embodiments, a polymeric biomaterial is or includes at least one polymer; in some embodiments, a polymer may be or comprise a copolymer. In some embodiments, a polymeric biomaterial is or comprises a preparation of at least two distinct polymer components (e.g., a preparation containing poloxamer and a second polymer component that is not a poloxamer). Those skilled in the art will be aware that certain polymers may exist and/or be available in a variety of forms (e.g., length, molecular weight, charge, topography, surface chemistry, degree and/or type of modification such as alkylation, acylation, quaternization, hydroxyalkylation, carboxyalkylation, thiolation, phosphorylation, glycosylation, etc.); in some embodiments, a preparation of such polymers may include a specified level and/or distribution of such form or forms. Additionally or alternatively, those skilled in the art will appreciate that, in some embodiments, one or more immunomodulatory properties of a polymeric biomaterial may be tuned by its biomaterial property(ies), including, e.g., surface chemistry of a polymeric biomaterial (e.g., modulated by hydrophobicity and/or hydrophilicity portions of a polymeric biomaterial, chemical moieties, and/or charge characteristics) and/or topography of a polymeric biomaterial (e.g., modulated by size, shape, and/or surface texture), for example as described in Mariani et al. “Biomaterials: Foreign Bodies or Tuners for the Immune Response?” International Journal of Molecular Sciences, 2019, 20, 636; the contents of which are incorporated herein in their entirety by reference for the purposes described herein.

Polymer network: The term “polymer network” is used herein to describe an assembly of polymer chains interacting with each other. In some embodiments, a polymer network forms a three-dimensional structure material. In some embodiments, a polymer network may be formed by linking polymer chains (“crosslinked polymer network”) using a crosslinker (e.g., as described herein). In some embodiments, a polymer network is transitioned from a precursor state when it is exposed to a temperature that is or above a critical gelation temperature, wherein the polymer network state has a viscosity materially above (e.g., at least 50% or above) that of the precursor state and the polymer network state comprises crosslinks not present in the precursor state. In some embodiments, a polymer network may be formed by non-covalent or non-ionic intermolecular association of polymer chains, e.g., through hydrogen bonding. In some embodiments, a polymer network may be formed by a combination of chemically crosslinking polymer chains and non-covalent or non-ionic intermolecular association of polymer chains.

Proinflammatory cytokine: As used herein, the term “proinflammatory cytokine” refers to a protein or glycoprotein molecule secreted by a cell (e.g., a cell of an immune system) that induces an inflammatory response. As will be appreciated by one of skilled in the art, inflammation may be immunostimulatory or immunosuppressive depending on the biological context.

Proinflammatory immune response: The term “proinflammatory immune response” as used herein refers to an immune response that induces inflammation, including, e.g., production of proinflammatory cytokines (including, e.g., but not limited to CXCL10, IFN-α, IFN-β, IL-1β, IL-6, IL-18, and/or TNF-alpha), increased activity and/or proliferation of Th1 cells, recruitment of myeloid cells, etc. In some embodiments, a proinflammatory immune response may be or comprise one or both of acute inflammation and chronic inflammation.

Proliferative disease: A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as matrix metalloproteinases (e.g., collagenases, gelatinases, and elastases); or 4) pathological angiogenesis as in proliferative retinopathy and tumor metastasis. Exemplary proliferative diseases include cancers (i.e., “malignant neoplasms”), benign neoplasms, angiogenesis or diseases associated with angiogenesis, inflammatory diseases, autoinflammatory diseases, and autoimmune diseases.

Prophylactically effective amount: A “prophylactically effective amount” is an amount sufficient to prevent (e.g., significantly delay onset or recurrence of one or more symptoms or characteristics of, for example so that it/they is/are not detected at a time point at which they would be expected absent administration of the amount) a condition. A prophylactically effective amount of a composition means an amount of therapeutic agent(s), alone or in combination with other agents, that provides a prophylactic benefit in the prevention of the condition. The term “prophylactically effective amount” can encompass an amount that improves overall prophylaxis or enhances the prophylactic efficacy of another prophylactic agent. Those skilled in the art will appreciate that a prophylactically effective amount need not be contained in a single dosage form. Rather, administration of an effective amount may involve administration of a plurality of doses, potentially over time (e.g., according to a dosing regimen).

Risk: As will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more. In some embodiments, risk may reflect one or more genetic attributes, e.g., which may predispose an individual toward development (or not) of a particular disease, disorder and/or condition. In some embodiments, risk may reflect one or more epigenetic events or attributes and/or one or more lifestyle or environmental events or attributes.

Salt: As used herein, the term “salt” refers to any and all salts and encompasses pharmaceutically acceptable salts.

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humor, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchoalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Small molecule: The term “small molecule” or “small molecule therapeutic” refers to a molecule, whether naturally occurring or artificially created (e.g., via chemical synthesis) that has a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, a small molecule is a therapeutically active agent such as a drug (e.g., a molecule approved by the U.S. Food and Drug Administration as provided in the Code of Federal Regulations (C.F.R.)). A small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, a small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, the contents of each of which are incorporated herein by reference for purposes described herein; such listed drugs are typically considered acceptable for use in accordance with the present disclosure.

Solvate: The term “solvate”, as used herein, has its art-understood meaning and refers to an aggregate of a compound (which may, for example, be a salt form of the compound) and one or more solvent atoms or molecules. In some embodiments, a solvate is a liquid. In some embodiments, a solvate is a solid form (e.g., a crystalline form). In some embodiments, a solid-form solvate is amenable to isolation. In some embodiments, association between solvent atom(s) and compound in a solvate is a non-covalent association. In some embodiments, such association is or comprises hydrogen bonding, van der Waals interactions, or combinations thereof. In some embodiments, a solvent whose atom(s) is/are included in a solvate may be or comprise one or more of water, methanol, ethanol, acetic acid, DMSO, THF, diethyl ether, and the like. Suitable solvates may be pharmaceutically acceptable solvates; in some particular embodiments, solvates are hydrates, ethanolates, or methanolates. In some embodiments, a solvate may be a stoichiometric solvate or a non-stoichiometric solvate.

Subject: A “subject” to which administration is contemplated includes, but is not limited to, a human (i.e., a male or female of any age group, e.g., a pediatric subject (e.g., infant, child, adolescent) or adult subject (e.g., young adult, middle-aged adult, or senior adult)) and/or a non-human animal, for example, a mammal (e.g., a primate (e.g., cynomolgus monkey, rhesus monkey); a domestic animal such as a cow, pig, horse, sheep, goat, cat, and/or dog; and/or a bird (e.g., a chicken, duck, goose, and/or turkey). In certain embodiments, the animal is a mammal (e.g., at any stage of development). In some embodiments, an animal (e.g., a non-human animal) may be a transgenic or genetically engineered animal. In some embodiments, a subject is a tumor resection subject, e.g., a subject who has recently undergone tumor resection. In some embodiments, a tumor resection subject is a subject who has undergone tumor resection in less than 72 hours (including, e.g., less than 48 hours, less than 24 hours, less than 12 hours, less than 6 hours, or lower) prior to receiving a composition described herein. In some embodiments, a tumor resection subject is a subject who has undergone tumor resection in less than 48 hours prior to receiving a composition described herein. In some embodiments, a tumor resection subject is a subject who has undergone tumor resection in less than 24 hours prior to receiving a composition described herein. In some embodiments, a tumor resection subject is a subject who has undergone tumor resection in less than 12 hours prior to receiving a composition described herein.

Substantially: As used herein, the term “substantially” refers to the qualitative condition of exhibiting total or near-total extent or degree of a characteristic or property of interest. Those skilled in the art will understand that an agent of interest, if ever, achieves or avoids an absolute result, e.g., an agent of interest that indeed has zero effect on an immune response, e.g., inflammation. The term “substantially” is therefore used herein to capture the potential lack of absoluteness inherent in many biological and chemical effects.

Sustained: As used interchangeably herein, the term “sustained” or “extended” typically refers to prolonging an effect and/or a process over a desirable period of time. For example, in the context of sustained immunomodulation (e.g., in the presence of a composition or preparation as described herein and/or utilized herein), such an immunomodulatory effect may be observed for a longer period of time after administration of oa particular immunomodulatory payload in the context of a composition comprising a biomaterial preparation and otherwise as described herein, as compared to that which is observed with administration of the same payload absent such a biomaterial preparation. In the context of sustained release of one or more agents of interest (e.g., one or more modulators of myeloid-derived suppressive cell function incorporated in biomaterial preparations described herein) from compositions described herein over a period of time, such release may occur on a timescale within a range of from about 30 minutes to several weeks or more. In some embodiments, the extent of sustained release or extended release can be characterized in vitro or in vivo. For example, in some embodiments, release kinetics can be tested in vitro by placing a preparation and/or composition described herein in an aqueous buffered solution (e.g., PBS at pH 7.4). In some embodiments, when a composition described herein is placed in an aqueous buffered solution (e.g., PBS at pH 7.4), less than 100% or lower (including, e.g., less than or equal to 90%, less than or equal to 80%, less than or equal to 70%, less than or equal to 50% or lower) of one or more agents of interest (e.g., one or more modulators of myeloid-derived suppressive cell function incorporated in biomaterial preparations described herein) is released within 3 hours from a biomaterial. In some embodiments, release kinetics can be tested in vivo, for example, by administering (e.g., implanting) a composition at a target site (e.g., mammary fat pad) of an animal subject (e.g., a mouse subject). In some embodiments, when a composition is administered (e.g., implanted) at a target site (e.g., mammary fat pad) of an animal subject (e.g., a mouse subject), less than or equal to 70% or lower (including, e.g., less than or equal to 60%, less than or equal to 50%, less than 40%, less than 30% or lower) of one or more agents of interest (e.g., one or more modulators of myeloid-derived suppressive cell function incorporated in biomaterial preparations described herein) is released in vivo 8 hours after the implantation.

Targeted agent: The term “targeted agent”, when used in reference to an anticancer agent means one that blocks the growth and spread of cancer by interfering with specific molecules (“molecular targets”) that are involved in the growth, progression, and/or spread of cancer. Targeted agents are sometimes called “targeted cancer therapies,” “molecularly targeted drugs,” “molecularly targeted therapies,” or “precision medicines.” Targeted agents differ from traditional chemotherapy in that targeted agents typically act on specific molecular targets that are specifically associated with cancer, and/or with a particular tumor or tumor type, stage, etc., whereas many chemotherapeutic agents act on all rapidly dividing cells (e.g., whether or not the cells are cancerous). Targeted agents are deliberately chosen or designed to interact with their target, whereas many standard chemotherapies are identified because they kill cells.

Tautomers: The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may be catalyzed by acid or base. Exemplary tautomerizations include keto-to-enol, amide-to-imide, lactam-to-lactim, enamine-to-imine, and enamine-to-(a different enamine) tautomerizations.

Test subject: As used herein, the term “test subject” refers to a subject to which technologies provided herein are applied for experimental investigation, e.g., to assess biomaterial degradation, and/or efficacy of compositions and/or preparations described herein in antitumor immunity. In some embodiments, a test subject may be a human subject or a population of human subjects. For example, in some embodiments, a human test subject may be a normal healthy subject. In some embodiments, a human test subject may be a tumor resection subject. In some embodiments, a test subject may be a mammalian non-human animal or a population of mammalian non-human animals. Non-limiting examples of such mammalian non-human animals include mice, rats, dogs, pigs, rabbits, etc., which in some embodiments may be normal healthy subjects, while in some embodiments may be tumor resection subjects. In some embodiments, mammalian non-human animals may be transgenic or genetically engineered animals.

Therapeutic agent: The term “therapeutic agent” refers to an agent having one or more properties that produce a desired, usually beneficial, physiological effect. For example, a therapeutic agent may treat, ameliorate, and/or prevent disease. Those skilled in the art, reading the present disclosure, will appreciate that the term “therapeutic agent”, as used herein, does not require a particular level or type of therapeutic activity, such as might be required for a regulatory agency to consider an agent to be “therapeutically active” for regulatory purposes. As will be understood by those skill in the art, reading the present disclosure, in some embodiments, certain biomaterial preparations described herein (in the absence of an immunomodulatory payload) may have one or more properties that contribute to and/or achieve a desired physiological effect, and therefore may be considered to be a “therapeutic agent” as that term is used here (whether or not such biomaterial would or would not be considered to be pharmaceutically active by any particular regulatory agency). In some embodiments, a therapeutic agent that may be utilized in preparations, compositions and/or methods described herein (e.g., involving biomaterial preparations described herein) may be or comprise an immunomodulatory payload. In some embodiments, a therapeutic agent that may be utilized in preparations, compositions and/or methods described herein (e.g., involving biomaterial preparations described herein) may be or comprise a non-immunomodulatory payload, e.g., comprising a biologic, a small molecule, nucleic acid, polypeptide, or a combination thereof. In some embodiments, a therapeutic agent that may be utilized in preparations, compositions and/or methods described herein (e.g., involving biomaterial preparations described herein) may be or comprise a chemotherapeutic agent, which in some embodiments may be or comprise a cytotoxic agent.

Therapeutically effective amount: A “therapeutically effective amount” is an amount sufficient to provide a therapeutic benefit in the treatment of a condition, which therapeutic benefit may be or comprise, for example, reduction in frequency and/or severity, and/or delay of onset of one or more features or symptoms associated with the condition. A therapeutically effective amount means an amount of therapeutic agent(s), alone or in combination with other therapies, that provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent. Those skilled in the art will appreciate that a therapeutically effective amount need not be contained in a single dosage form. Rather, administration of an effective amount may involve administration of a plurality of doses, potentially over time (e.g., according to a dosing regimen, and particularly according to a dosing regimen that has been established, when applied to a relevant population, to provide an appropriate effect with a desired degree of statistical confidence).

Temperature-responsive: As used herein, the term “temperature-responsive”, in the context of a temperature-responsive polymer or biomaterial (e.g., a polymeric biomaterial), refers to a polymer or biomaterial (e.g., polymeric biomaterial) that exhibits an instantaneous or discontinuous change in one or more of its properties at a critical temperature (e.g., a critical gelation temperature). For example, in some embodiments, one or more of such properties is or comprise a polymer's or biomaterial's solubility in a particular solvent. By way of example only, in some embodiments, a temperature-responsive polymer or biomaterial (e.g., polymeric biomaterial) is characterized in that it is a homogenous polymer solution or colloid that is stable below a critical temperature (e.g., a critical gelation temperature) and instantaneously form a polymer network (e.g., a hydrogel) when the critical temperature (e.g., critical gelation temperature) has been reached or exceeded. In some embodiments, a temperature-responsive polymer or biomaterial (e.g., polymeric biomaterial) may be temperature-reversible, e.g., in some embodiments where a polymer solution may instantaneously form a polymer network at a temperature of or above a critical gelation temperature, and such a resulting polymer network may instantaneously revert to a homogenous polymer solution when the temperature is reduced to below the critical gelation temperature.

Treat: The terms “treatment,” “treat,” and “treating” refer to reversing, alleviating, delaying the onset of, or inhibiting the progress of a “pathological condition” (e.g., a disease, disorder, or condition, including one or more signs or symptoms thereof) described herein, e.g., cancer or tumor. In some embodiments, treatment may be administered after one or more signs or symptoms have developed or have been observed. Treatment may also be continued after symptoms have resolved, for example, to delay or prevent recurrence and/or spread.

Tumor: The terms “tumor” and “neoplasm” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An example of a pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

Tumor removal: As used herein, the term “tumor removal” encompasses partial or complete removal of a tumor, which may be resulted from a cancer therapy, e.g., surgical resection. In some embodiments, tumor removal refers to physical removal of part or all of a tumor by surgery (i.e., “tumor resection”). In some embodiments, tumor removal may be resulted from a surgical tumor resection and an adjuvant therapy (e.g., chemotherapy, immunotherapy, and/or radiation therapy). In some embodiments, an adjuvant therapy may be administered after a surgical tumor resection, e.g., at least 24 hours or more after a surgical tumor resection.

Tumor resection subject: As used herein, the term “tumor resection subject” refers to a subject who is undergoing or has recently undergone a tumor resection procedure. In some embodiments, a tumor resection subject is a subject who has at least 70% or more (including, at least 80%, at least 90%, at least 95%, at least 98%, at least 99%, or higher (including 100%) of gross tumor mass removed by surgical resection. Those of skill in the art will appreciate that, in some cases, there may be some residual cancer cells microscopically present at a visible resection margin even though gross examination by the naked eye shows that all of the gross tumor mass has been apparently removed. In some embodiments, a tumor resection subject may be determined to have a negative resection margin (i.e., no cancer cells seen microscopically at the resection margin, e.g., based on histological assessment of tissues surrounding the tumor resection site). In some embodiments, a tumor resection subject may be determined to have a positive resection margin (i.e., cancer cells are seen microscopically at the resection margin, e.g., based on histological assessment of tissues surrounding the tumor resection site). In some embodiments, a tumor resection subject may have micrometastases and/or dormant disseminated cancer cells that can be driven to progress/proliferate by the physiologic response to surgery. In some embodiments, a tumor resection subject receives a composition (e.g., as described and/or utilized herein) immediately after the tumor resection procedure is performed (e.g., intraoperative administration). In some embodiments, a tumor resection subject receives a composition (e.g., as described and/or utilized herein) postoperatively within 24 hours or less, including, e.g., within 18 hours, within 12 hours, within 6 hours, within 3 hours, within 2 hours, within 1 hour, within 30 mins, or less.

Tumor resection site: The term “tumor resection site” generally means a site in which part or all of a tumor was or is being removed through tumor resection. In some embodiments, the term “tumor resection site” refers to a site in which at least 70% or more (including, at least 80%, at least 90%, at least 95%, at least 98%, at least 99%, or higher (including 100%) of gross tumor mass is removed by surgical resection. Those of skill in the art will appreciate that, in some cases, there may be some residual cancer cells microscopically present at a visible resection margin even though gross examination by the naked eye shows that all of the gross tumor mass has been apparently removed. In some embodiments, a tumor resection site may be determined to have a negative resection margin (i.e., no cancer cells seen microscopically at the resection margin, e.g., based on histological assessment of tissues surrounding the tumor resection site). In some embodiments, a tumor resection site may be determined to have a positive resection margin (i.e., cancer cells are seen microscopically at the resection margin, e.g., based on histological assessment of tissues surrounding the tumor resection site).

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant biomaterial (e.g., a variant polymer or a polymeric biomaterial comprising a variant polymer) may differ from a reference biomaterial (e.g., a reference polymer or polymeric biomaterial) as a result of one or more structural modifications (e.g., but not limited to, additions, deletions, and/or modifications of chemical moieties, and/or grafting) provided that the variant biomaterial (e.g., variant polymer or polymeric biomaterial comprising such a variant polymer) can retain the desired property(ies) and/or function(s) (e.g., immunomodulation and/or temperature-responsiveness) of the reference biomaterial. For example, a variant of an immunomodulatory biomaterial may differ from a reference immunomodulatory biomaterial (e.g., a reference polymer or polymeric biomaterial) as a result of one or more structural modifications (e.g., but not limited to, additions, deletions, and/or modifications of chemical moieties, and/or grafting) provided that the variant biomaterial (e.g., variant polymer or polymeric biomaterial comprising such a variant polymer) can act on an immune system (e.g., by stimulating innate immunity), e.g., when used in a method described herein. In some embodiments, a variant immunomodulatory biomaterial (e.g., a variant polymer or a polymeric biomaterial comprising a variant polymer) is characterized in that, when assessed at 24 hours after administration of such a variant immunomodulatory biomaterial (e.g., a variant polymer or a polymeric biomaterial comprising a variant polymer) to a target site in a subject, an amount of one or more proinflammatory cytokines (e.g., but not limited to CXCL10, IFN-α, IFN-β, IL-1β, IL-6, IL-18, and/or TNF-α) observed at the target site and/or body circulation of the subject is at least 60% or more (e.g., including, e.g., at least 70%, at least 80%, at least 90%, at least 95%, at least 98%, or up to 100%) of that observed when a reference biomaterial (e.g., a reference polymer or polymeric biomaterial) is administered at the target site. In some embodiments, a variant immunomodulatory biomaterial (e.g., a variant polymer or a polymeric biomaterial comprising a variant polymer) is characterized in that, when assessed at 24 hours after administration of such a variant biomaterial (e.g., a variant polymer or a polymeric biomaterial comprising a variant polymer) to a target site in a subject, an amount of one or more proinflammatory cytokines (e.g., but not limited to CXCL10, IFN-α, IFN-β, IL-1β, IL-6, IL-18, and/or TNF-α) observed at the target site and/or body circulation of the subject is at least 1.1-fold or more (e.g., including, e.g., at least 1.5-fold, at least 2-fold, at least 3-fold, at least 4-fold, at least 5-fold, or more) of that observed when a reference biomaterial (e.g., a reference polymeric biomaterial) is administered at the target site.

In some embodiments, a variant biomaterial (e.g., a variant polymeric biomaterial) exhibits at least one physical characteristic that is different from that of a reference biomaterial (e.g., a reference polymeric biomaterial). For example, in some embodiments, a variant biomaterial (e.g., a variant polymeric biomaterial) can exhibit increased water solubility (e.g., at a physiological pH) as compared to that of a reference biomaterial (e.g., a reference polymeric biomaterial). In some embodiments, a variant has 10, 9, 8, 7, 6, 5, 4, 3, 2, or 1 structural modifications as compared with a reference. In some embodiments, a variant has a small number (e.g., fewer than 5, 4, 3, 2, or 1) number of structural modifications (e.g., alkylation, acylation, quaternization, hydroxyalkylation, carboxyalkylation, thiolation, phosphorylation, glycosylation, etc.). In some embodiments, a variant has not more than 5, 4, 3, 2, or 1 additions or deletions of chemical moieties, and in some embodiments has no additions or deletions, as compared with a reference. In some embodiments, a variant is an entity that can be generated from a reference by chemical manipulation. In some embodiments, a variant is an entity that can be generated through performance of a synthetic process substantially similar to (e.g., sharing a plurality of steps with) one that generates a reference.

### DETAILED DESCRIPTION OF CERTAIN EMBODIMENTS

The present disclosure, among other things, provide technologies, including, e.g., compositions each comprising a biomaterial preparation and a modulator of myeloid-derived suppressive cell function (e.g., a modulator of neutrophil function) and methods of uses, that may be particularly useful and/or may provide particular beneficial effects, e.g., as described herein. In some embodiments, such compositions are particularly useful for monotherapy. In some embodiments, such compositions may be useful for combination therapies.

Among other things, the present disclosure provides an insight that local modulation of recruitment, survival, and/or immune effector function of immune cells following resection can be particularly useful and/or may provide particular beneficial effects, e.g., as described herein.

In certain aspects, without wishing to be bound by a particular theory, the present disclosure observes that inflammatory changes that occur at a surgical tumor resection can induce recruitment of numerous immune and/or inflammatory cell types and/or the release of humoral factors, thus promoting tumor capture and growth; moreover, recruited immune cells (e.g., MDSCs, neutrophils and/or macrophages) can secrete factors (e.g., VEGF and matrix metalloproteinases (MMPs)) that are known to promote growth and/or dissemination of cancer; see, e.g., Hiller et al. “Perioperative events influence cancer recurrence risk after surgery” Nature Reviews: Clinical Oncology (2018) 15: 205-218; and Tohme et al. “Surgery for Cancer: A Trigger for Metastases” Cancer Research (2017) 77: 1548-1552; the contents of each of which are incorporated herein in their entirety by reference for the purposes described herein. In further aspects, without wishing to be bound by a particular theory, the present disclosure observes that recruited neutrophils may react to injured tissues around a tumor resection site, for example, by forming neutrophil extracellular traps that facilitate entrapment and accumulation of circulating tumor cells; moreover, such web-like DNA neutrophil extracellular traps may contain a variety of molecules (e.g., proinflammatory molecules) that are useful for capture of tumor cells and/or augmented growth of metastases in surgically manipulated sites. See id.

In some embodiments, the present disclosure, among other things, provides an insight that intraoperative modulation of neutrophil immune effector functions at a tumor resection site may be particularly useful and/or effective for cancer treatment. In some embodiments, such modulation may be useful and/or effective to reduce tumor relapse and/or regrowth. In some embodiments, such modulation may be useful and/or effective to reduce tumor metastasis. Indeed, in some embodiments, the present disclosure, among other things, teaches that intraoperative administration of a combination of a biomaterial (e.g., polymeric biomaterial) and a modulator of myeloid-derived suppressor cells (MDSCs) and, more particularly a combination of a biomaterial (e.g., polymeric biomaterial) and a modulator of neutrophils as described herein, at a target site (e.g., a tumor resection site) can provide beneficial therapeutic effects (e.g., ones as described herein). In some embodiments, such modulators of MDSCs and more particularly neutrophils that are useful for technologies described herein can inhibit recruitment and/or survival of such immune cells. Additionally or alternatively, in some embodiments, such modulators of MDSCs and more particularly neutrophils that are useful for technologies described herein can modulate effector function, e.g., in some embodiments inhibit production of certain pro-tumorigenic factors and/or in some embodiments induce production of certain anti-tumorigenic factors.

In some aspects, provided are methods comprising intraoperatively administering at a target site (e.g., at or near a tumor resection site) of a subject suffering from cancer, a composition comprising a biomaterial (e.g., polymeric biomaterial) and a modulator of myeloid-derived suppressive cells (e.g., MDSCs, neutrophils, macrophages, monocytes, etc.).

In some embodiments, the present disclosure provides compositions that can localize delivery of one or more modulators of myeloid-derived suppressive cells such as modulators of MDSCs and/or more particularly modulators of neutrophils to a target site (e.g., at or near a site at which a tumor has been removed and/or cancer cells have been treated or killed, e.g., by chemotherapy or radiation) and thereby concentrate the action of such modulators to a target site in need thereof. Such compositions can be particularly useful for treating cancer. In particular, compositions described herein may deliver one or more therapeutic agents that act on (e.g., modulate) one or more attributes of MDSCs and/or neutrophils such as recruitment, survival, and/or immune effector function of neutrophils, e.g., following a tumor resection, for the treatment of cancer, such as, for example, by preventing (e.g., delaying onset of, reducing extent of) tumor recurrence and/or metastasis, in some embodiments while minimizing adverse side effects and/or systemic exposure.

### I. Provided Compositions

In some embodiments, the present disclosure, among other things, provides compositions comprising a biomaterial preparation (e.g., ones described herein) and at least one (including, e.g., at least two, at least three, at least four or more) modulator of immune effector cell function and more particularly at least one (including, e.g., at least two, at least three, at least four or more) modulator of myeloid-derived suppressive cell function. In some embodiments, a composition comprises a biomaterial preparation (e.g., ones described herein) and a single modulator of myeloid-derived suppressive cell function. In many embodiments, a modulator of immune cell function (e.g., a modulator of myeloid-derived suppressive cell function) is administered in an amount that is effective to inhibit recruitment, survival, proliferation, and/or effector function of myeloid-derived suppressive cells (e.g., neutrophils). Therefore, in some embodiments, modulators described herein may be administered in an amount that is higher than what is typically used in other therapeutic context. In some embodiments, modulators described herein may be administered in an amount that is lower than what is typically used in other therapeutic context. In some embodiments, a composition comprising or consisting of a biomaterial preparation and a single modulator of myeloid-derived suppressive cell function described herein is particularly useful for cancer treatment as monotherapy following tumor resection in the absence of any other therapeutic agents to be included in the composition. In some embodiments, such a composition may comprise one or more additional therapeutic agents.

### Exemplary Modulators of Myeloid-Derived Suppressor Cells (MDSCs) and Neutrophils

In some embodiments, a modulator of immune effector cell to be present in a composition described herein is or comprises a modulator of a myeloid-derived suppressor cell (MDSC). MDSCs typically refer to a heterogeneous population of myeloid cells that possess immune suppressive capacity, which include granulocytic or polymorphonuclear MDSCs (g-MDSCs or PMN-MDSCs) and monocytic MDSCs (m-MDSCs). These cells are thought to have inhibitory effects on lymphocytes and lymphocyte proliferation. MDSCs have been shown to accumulate in the circulation when tumors are present, and MDSC numbers generally correlate with an inferior prognosis. Thus, MDSCs have been thought to be one of the drivers of not only cancer-associated immune invasion but also tumor progression and metastasis by suppressing anti-tumor immune responses such as, e.g., in some embodiments by reducing or inhibiting proliferative and/or activation capacity of T cells; See e.g., Kumar et al., “The nature of myeloid-derived suppressor cells in the tumor microenvironment” Trends Immunology (2016) 37(3): 208-220; the contents of which are incorporated herein in their entirety by reference for the purposes described herein.

Typically, g-MDSCs or PMN-MDSCs and neutrophils (e.g., mature neutrophils) share similar morphology and expression of cell surface markers, whereas m-MDSCs are similar to monocytes. For example, mature neutrophils can be defined by a CD14(−), CD15(+), CD66b(+), CD16(+) pattern of cell-surface protein expression while PMN-MDSCs are mostly referred to as CD14(−), CD15(+), CD66b(+), CD16(+), CD11b(+), CD33(+), HLA-DR. Because of the similarities between g-MDSCs or PMN-MDSCs and neutrophils (e.g., mature neutrophils) in phenotype and morphology and recent indication that neutrophils are able to exert immune suppressive capacity in certain biological context, one of skill in the art will appreciate that neutrophils under certain biological context may be considered as MDSCs, e.g., in some embodiments where certain neutrophils exhibit immune suppressive capacity as MDSCs. Accordingly, in some embodiments, a modulator of a myeloid-derived suppressor cell (MDSC) described herein may be useful and/or effective as a modulator of neutrophils; See e.g., Shaul & Fridlender “Tumour-associated neutrophils in patients with cancer” Nature Reviews: Clinical Oncology (2019) 16: 601-620; the contents of which are incorporated herein in their entirety by reference for the purposes described herein.

Neutrophils are the most abundant cell type among circulating white blood cells and form the first line of defense against invading pathogens as part of the innate immune response. Neutrophils are remarkably versatile polymorphonuclear cells, which functions include but are not limited to phagocytosis and killing. For example, in some embodiments, neutrophils are involved in primary defense against infections via, for example, phagocytosis, generation of cytotoxic molecules, release of cytotoxic enzymes and/or formation of neutrophil extracellular traps that typically contain extracellular extrusion of web-like DNA to entrap circulating tumor cells. In some embodiments, neutrophils can play a role in the regulation and/or cascading development of inflammatory and/or immune responses. In some embodiments, neutrophils can modulate immune response through production and/or recognition of various cytokines and/or chemokines.

Circulating tumor-associated neutrophils (TANs) are reported to be able to retain some functional plasticity and can undergo “alternative activation” to confer antitumor properties (e.g., cytotoxicity toward tumor cells and/or inhibition of metastasis) or pro-tumor progression properties (e.g., angiogenic switch, stimulating tumor cell motility, migration, and/or invasion) when exposed to various cues found in a tumor micro environment (TME). For example, the presence of transforming growth factor-O (TGFβ) has been demonstrated to promote a pro-tumor phenotype (N2-like phenotype), whereas the presence of interferon-0 (IFNβ) or the inhibition of TGFβ signaling results in TANs of an antitumor phenotype (N1-like phenotype) See e.g., Fridlender et al., “Polarization of Tumor-Associated (TAN) Phenotype by TGFβ: “N1” versus “N2” TAN” Cancer Cell (2009) 16(3): 183-194; and Granot “Neutrophils as a Therapeutic Target in Cancer” frontiers in Immunology (2019) 10:1710; the contents of each of which are incorporated herein in their entirety by reference for the purposes described herein.

In some embodiments, modulators of MDSCs and more particularly neutrophils that are useful for technologies described herein can inhibit recruitment and/or survival of such immune cells. Additionally or alternatively, such modulators of MDSCs and more particularly neutrophils that are useful for technologies described herein can modulate effector function, e.g., in some embodiments inhibit production of certain pro-tumorigenic factors and/or in some embodiments induce production of certain anti-tumorigenic factors.
A) Inhibiting Recruitment, Survival, and/or Proliferation of MDSCs and/or Neutrophils

In some embodiments, a composition described herein comprises a biomaterial (e.g., polymeric biomaterial) and a modulator of MDSCs, and more particularly, a modulator of neutrophils, that modulates their chemotaxis and/or recruitment. In some embodiments, such a modulator of neutrophils and/or MDSCs is or comprises an inhibitor of neutrophil and/or MDSC chemotaxis and/or recruitment. In some embodiments, compositions described herein are useful to inhibit recruitment of neutrophils and/or MDSCs to a tumor resection site.

In some embodiments, a modulator of MDSC/neutrophil recruitment is or comprises an inhibitor of colony stimulating factor 1 (CSF-1) and/or CSF-1 Receptor (CSF-1R) signaling. Without being bound by a particular theory, it is thought that neutrophils are a major source of CSF-1 and CSF-1R and that limiting the production of these molecules reduces immune cell chemotaxis; see e.g., Tang et al., “Neutrophil and Macrophage Cell Surface Colony-Stimulating Factor 1 Shed by ADAM17 Drive mouse Macrophage Proliferation in Acute and Chronic Inflammation” Mol Cell Biol (2018) 38(17): e00103-18; and Cannarile et al., “Colony-stimulating factor 1 receptor (CSF1R) inhibitors in cancer therapy” Journalfor Immuno Therapy of Cancer (2017) 5, 53; and Xun et al., “Small-Molecule CSF1R Inhibitors as Anticancer Agents” Curr Med Chem. (2020) 27(23):3944-3966; the contents of each of which are incorporated herein in their entirety by reference for the purposes described herein. In some embodiments, an inhibitor of CSF-1/CSF-1R signaling can be or comprise pexidartinib (PLX3397), Linifanib (ABT-869), OSI-930, CEP-32496 (RXDX-105), Ki20227, PLX5622, MCS-110, FPA008, RG7155, IMC-CS4, AMG820, UCB6352, GW2580, BLZ945, edicotinib, or any combinations thereof.

In some embodiments, a modulator of MDSC/neutrophil recruitment may be or comprise an inhibitor of interleukin 34 (IL-34) signaling. In some embodiments, such inhibitors may be directed to IL-34. In some embodiments, such inhibitors may be directed to an IL-34 receptor (e.g., colony stimulated factor 1 receptor (CSF-1R) and/or protein-tyrosine phosphatase ξ(PTP-ξ). Without being bound by a particular theory, it is thought that IL-34 signaling promotes neutrophil recruitment; see e.g., Baek et al. “IL-34 mediates acute kidney injury and worsens subsequent chronic kidney disease” The Journal of Clinical Investigation 125(8):3198-3214; the contents of which are incorporated herein in their entirety by reference for the purposes described herein. In some embodiments, an inhibitor of IL-34 signaling may be or comprise an anti-IL-34 antibody, an anti-CSF-1R antibody, an anti-PTP-ξ antibody, or any combination thereof.

In certain embodiments, a modulator of MDSC/neutrophil recruitment may be or comprise an inhibitor of a CD47-signal regulatory protein alpha (SIRPα) signaling pathway. While not being bound by a particular theory, it is thought that CD47-SIRPα signaling may promote mobility of MDSC/neutrophils, while inhibition of such a signaling may reduce their mobility. In certain embodiments, inhibitors of a CD47-SIRPα signaling pathway may be or comprise but are not limited to: Hu5F9-G4, IB1188, SRF231, TTI-621, CC-90002, or any combination thereof.

In certain embodiments, a modulator of MDSC/neutrophil recruitment is or comprises an inhibitor of macrophage migration inhibitory factor (MIF)/CD74 signaling. In certain embodiments, inhibitors of a MIF/CD74 signaling pathway can be or comprise but are not limited to: Orita-13, anti-CD74 monoclonal antibodies, BTZO-1, ISO-1, Alam-4b, ISO-66, Jorgensen-3g, Jorgensen 3h, Dziedzic-3bb (Cisneros-3i), Cisneros-3j, 4-IPP, BITC, NVS-2, MIF098 (Alissa-5), K664-1, T-614, Kok-10, Kok-17, CPSI-2705, CPSI-1306, SCD-19, or any combination thereof; See e.g., Kok et al., “Small molecule inhibitors of macrophage migration inhibitory factor (MIF) as emerging class of therapeutics for immune disorders” Drug Discovery Today (2018), 23(11): 1910-1918; the contents of which are incorporated herein in their entirety by reference for the purposes described herein.

In certain embodiments, a modulator of MDSC/neutrophil recruitment may be or comprise an inhibitor of one or more C-C motif chemokine signaling pathways and/or C-X-C motif chemokine signaling pathway. In certain embodiments, an inhibitor of MDSC/neutrophil recruitment may be an inhibitor of: a CCL2/CCR2 signaling pathway, CCL3/CCR1 signaling pathway, CCL3/CCR4 signaling pathway, CCL3/CCR5 signaling pathway, CCL4/CCR5 signaling pathway, CCL4/CCR8 signaling pathway, CCL5/CCR1 signaling pathway, CCL5/CCR3 signaling pathway, CCL5/CCR5 signaling pathway, CCL8/CCR1 signaling pathway, CCL8/CCR2 signaling pathway, CCL8/CCR3 signaling pathway, CCL8/CCR5 signaling pathway, and/or CXCL12/CXCR4 signaling pathway. In some embodiments, such inhibitors may be directed to CCR1, CCR2, CCR2B, CCR3, CCR4, CCR5, CCR8, CXCR2, CXCR4, and/or combinations thereof. In certain embodiments, such inhibitors may be directed to CCL2, CCL3, CCL4, CCL5, CCL8, CXCL12, and/or combinations thereof. In some embodiments, such inhibitors may be directed to one or more neutrophil-derived chemokines including, e.g., but not limited to CXCL1, CXCL2, CXCL3, CXCL4, CXCL5, CXCL6, CXCL8, CXCL9, CXCL10, CXCL11, CXCL12, CXCL13, CXCL15, CCL2, CCL3, CCL4, CCL5, CCL7, CCL9, CCL12, CCL17, CCL18, CCL19, CCL20, CCL22, and/or combinations thereof. See, e.g., Tecchio and Cassatella, “Neutrophil-derived chemokines on the road to immunity” Seminars in Immunology (2016) 28:119-128; which is incorporated herein in its entirety by reference for the purposes described herein.

In certain embodiments, a modulator of MDSC/neutrophil recruitment may be or comprise an inhibitor of CCR2, CCR5, CXCR2, CXCR4, CXCL12, and/or CCL2. In certain embodiments, the inhibitor of a MDSC/neutrophil recruitment may be or comprise an inhibitor of CCR5, CXCR2, CXCL12, and/or CCL2.

In certain embodiments, a modulator of MDSC/neutrophil recruitment may be or comprise an inhibitor of CCR2. Without being bound by a particular theory, CCR2 is thought to be essential for neutrophil tissue infiltration; see e.g., Souto et al., “Essential role of CCR2 in neutrophil tissue infiltration and multiple organ dysfunction in sepsis” Am J Respir Crit Care Med. (2011): 183(2): 234-242; the contents of which are incorporated herein in their entirety by reference for the purposes described herein. In certain embodiments, an inhibitor of CCR2 signaling pathway may be or comprise but is not limited to: PF-04136309, CCX872-B, MLN1202, BMS-813160, BMS CCR2 22, MK-0812, plozalizumab, or any combination thereof.

In certain embodiments, a modulator of MDSC/neutrophil recruitment and/or function may be an inhibitor of CCR5. While not being bound by a particular theory, it is thought that CCR5 facilitates the release of immature neutrophils from bone marrow and their recruitment to tumorigenic tissues. In certain embodiments, an inhibitor of CCR5 signaling pathway may be or comprise but is not limited to: maraviroc, DAPTA, GSK706769, INCB009471, GW873140, Vicriviroc, PRO 140, or any combination thereof.

In certain embodiments, a modulator of MDSC/neutrophil recruitment may be or comprise an inhibitor of CCR2 and CCR5. In certain embodiments, an inhibitor of CCR2 and CCR5 signaling pathway may be or comprise but is not limited to: PF-04634817, cenicriviroc, BMS-813160, or any combination thereof.

In certain embodiments, a modulator of MDSC/neutrophil recruitment may be or comprise an inhibitor CXCR4/CXCL12 signaling. While not being bound by a particular theory, CXCR4 is thought to function as a master regulator of neutrophil trafficking in health and disease; see e.g., Filippo and Rankin “CXCR4, the master regulator of neutrophil trafficking in homeostasis and disease” European Jof ClinicalInvestigation (2018); the contents of which are incorporated herein in their entirety by reference for the purposes described herein. In certain embodiments, an inhibitor of CXCR4/CXCL12 mediated signaling may be or comprise but is not limited to: plerixafor (AMD-3100), an anti-CXCR4 antibody (e.g., ulocuplumab), Burixafor (TG-0054), TG0054, AMDO70, AMD3465, AMD11070, LY2510924, MSX-122, CTCE-9908, POL6326, CX-01, X4P-001, BL-8040, USL311, SPOlA, or any combination thereof.

In certain embodiments, a modulator of MDSC/neutrophil recruitment may be or comprise an inhibitor of CCL2. While not being limited by a particular theory, CCL2 is thought to mediate neutrophil recruitment, promote cancer metastasis, and/or promote angiogenesis; see e.g., Reichel et al., “Ccl2 and Ccl3 mediate neutrophil recruitment via induction of protein synthesis and generation of lipid mediators” Arterioscler Thromb Vasc Biol. (2009) 29(11): 1787-93; and Bonapace et al., “Cessation of CCL2 inhibition accelerates breast cancer metastasis by promoting angiogenesis” Nature (2014) 515, 130-133; and Mora et al., “Bindarit: an anti-inflammatory small molecule that modulates the NFκB pathway” Cell Cycle (2012) 11(1) 159-169; the contents of each of which are incorporated herein in their entirety by reference for the purposes described herein. In certain embodiments, an inhibitor of CCL2 may be or comprise bindarit.

In certain embodiments, a modulator of MDSC/neutrophil recruitment may be or comprise an inhibitor of CXCR2 and/or CXCR2 ligands. While not being limited by a particular theory, CXCR2 is thought to localize neutrophils to tumors, attenuate granulocytosis, and increase vascular permeability; see e.g., Zarbock et al., “Therapeutic inhibition of CXCR2 by Reparixin attenuates acute lung injury in mice” British Journal of Pharmacology (2008): 155(3): 357-364; the contents of which are incorporated herein in their entirety by reference for the purposes described herein. In certain embodiments, an inhibitor of CXCR2 mediated signaling may be or comprise but is not limited to: Reparixin, Navarixin, Danirixin, AZD5069, DF2156A, SB-656933, QBM076, SB225002, Humax IL8, ABX-IL8, Ladarixin, SX-682, or any combination thereof.

In certain embodiments, a modulator of MDSC/neutrophil recruitment can be or comprise an inhibitor of CXCL1 mediated signaling pathways. In certain embodiments, an inhibitor of CXCL1 mediated signaling pathways can be but is not limited to: a small molecule, an oligonucleotide, a polypeptide and/or a protein. In certain embodiments, an inhibitor of CXCL1 can be or comprise an anti-CXCL1 neutralizing antibody.

In certain embodiments, a modulator of MDSC/neutrophil recruitment can be or comprise an inhibitor of a NF-κB signaling pathway. While not being bound by a particular theory, it is thought that NF-κB signaling may be necessary for CXCL1, CXCL2 and/or CXCL8 expression and/or subsequent neutrophil recruitment. In certain embodiments, an inhibitor of NF-κB mediated signaling pathways can be or comprise but is not limited to: Bithionol, Bortezomib, Cantharidin, Chromomycin A3, Daunorubicinum, Digitoxin, Ectinascidin 743, Emetine, Fluorosalan, Manidipine hydrochloride, Narasin, Lestaurtinib, Ouabain, Sorafenib tosylate, Sunitinib malate, Tioconazole, Tribromsalan, Triclabendazolum, Zafirlukast, BAY11-7082, or any combinations thereof.

In certain embodiments, a modulator of MDSC/neutrophil recruitment may be or comprise an inhibitor of Janus kinase (JAK) related signaling pathways. While not being bound by a particular theory, it is thought that inhibition of JAK reduces CXCL1 expression and can improve efficacy of allergen-specific immunotherapy for conditions such as asthma. In certain embodiments, an inhibitor of JAK mediated signaling pathways may be or comprise but is not limited to: Ruxolitinib (INC424), Tofacitinib (CP-690,550), INCB052793, AZD4205, TD-1473, Givinostat (ITF2357), Pacritinib, Decemotinib (VS-509), Baricitinib, Lestauritinib (CEP-701), BMS-911543, or any combination thereof.

In certain embodiments, a modulator of MDSC/neutrophil recruitment may be or comprise an inhibitor of mitogen-activated protein kinase (MEK) signaling. While not being bound by a particular theory, it is thought that MEK inhibition inhibits CXCL1-induced ERK1/2 phosphorylation, which may lead to reduced cellular proliferation. In certain embodiments, such an inhibitor may be or comprise PD98059 and/or U0126.

In certain embodiments, a modulator of MDSC/neutrophil recruitment can be or comprise an inhibitor of inhibitor of nuclear factor kappa-B kinase (IKK) signaling. While not being bound by a particular theory, it is thought that IKK inhibition may decrease CXCL1, CXCL2, and/or CXCL8 production, potentially suppressing clonogenic growth of cancer cells. In certain embodiments, an inhibitor of MDSC/neutrophil recruitment acting through TKK related signaling pathways can be or comprise TPCA-1, IKK16, Bay65-1942, or any combination thereof.

In some embodiments, a modulator of MDSC/neutrophil recruitment may be or comprise an inhibitor of a TGFβ signaling pathway. While not being bound by a particular theory, TGFβ is thought to function as a potent MDSC/neutrophil chemoattractant, and in some embodiments, a modulator of MDSC/neutrophil recruitment may be or comprise an inhibitor of TGFβ; see e.g., Reibman et al., “Transforming growth factor beta 1, a potent chemoattractant for human neutrophils, bypasses classic signal-transduction pathways” Proc Natl Acad Sci USA (1991) 88(15): 6805-6809; and Brandes et al., “Type I transforming growth factor-beta receptors on neutrophils mediate chemotaxis to transforming growth factor-beta” Journal of Immunology (1991) 147(5): 1600-1606; the contents of each of which are incorporated herein in their entirety by reference for the purposes described herein. In some embodiments, compositions described herein may comprise TGFβ signaling pathway inhibitors including but not limited to: TGFβR1 kinase inhibitors (e.g., galunisertib), and/or TGFβ signaling pathway inhibitors (e.g., vactosertib, RepSox, GW788388, LY364947, SB505124, SB525334, K02288, and/or LDN-193189). In some embodiments, a TGFβ signaling pathway inhibitor may be or comprise an anti-TGFβ antibody (e.g., fresolomumab).

In certain embodiments, a modulator of MDSC/neutrophil recruitment can or comprises an inhibitor of low-molecular mass protein-7 (LMP7). While not being bound by a particular theory, it is thought that LMP7 inhibition may reduce CXCL1, CXCL2, and/or CXCL3 expression. In some embodiments, an inhibitor of LMP7 can be or comprise ONX-0914.

In certain embodiments, an inhibitor of MDSC/neutrophil recruitment can be or comprise one or more inhibitors of at least two or more (including, e.g., at least three, at least four more) cytokines and/or chemokines described herein. While not being bound by a particular theory, it is thought that general and/or multiple cytokine inhibition can decrease the accumulation and/or recruitment of neutrophils. In certain embodiments, such an inhibitor can be or comprise a cytokine release inhibitor, e.g., JTE-607.

In certain embodiments, a modulator of MDSC/neutrophil recruitment can be or comprise an inhibitor of neddylation. While not being bound by a particular theory, it is thought that neddylation promotes CXCL1 production and may inhibit cellular apoptosis. In certain embodiments, an inhibitor of neddylation can be or comprise MLN4924.

In certain embodiments, a modulator of MDSC/neutrophil recruitment can be or comprise an inhibitor of protein kinase C zeta (PKCξ). While not being bound by a particular theory, it is thought that PKC(promotes CXCL1 production. In certain embodiments, an inhibitor of PKC(can be or comprise MA130.

In certain embodiments, a modulator of MDSC/neutrophil recruitment can be or comprise an inhibitor of c-Jun N-terminal kinase (INK). While not being bound by a particular theory, it is thought that JNK signaling promotes CXCL1 and/or CXCL2 expression. In certain embodiments, an inhibitor of INK signaling can be or comprise SP600125.

In certain embodiments, a modulator of MDSC/neutrophil recruitment can be or comprise an inhibitor of purinergic receptor P2Y12 (P2YR12). While not being bound by a particular theory, it is thought that P2YR12 signaling promotes CXCL1 expression and release. In certain embodiments, an inhibitor of P2Y12 receptors can be or comprise PSB0739.

In certain embodiments, a modulator of MDSC/neutrophil recruitment can be or comprise an inhibitor of nicotinamide phosphoribosyltransferase (NAMPT). While not being bound by a particular theory, it is thought that NAMPT promotes CXCL1 and/or CXCL2 expression and release. In certain embodiments, an inhibitor of NAMPT can be or comprise FK866.

In certain embodiments, a modulator of MDSC/neutrophil recruitment can be or comprise an inhibitor of protein tyrosine kinase (PTK). While not being bound by a particular theory, it is thought that PTK signaling promotes CXCL1 expression and release. In certain embodiments, an inhibitor of PTK can be or comprise PP2.

In certain embodiments, a modulator of MDSC/neutrophil recruitment may be or comprise an inhibitor of the proteasome. While not being bound by a particular theory, it is thought that proteasome mediated protein degradation can promote CXCL1 expression and release. In certain embodiments, an inhibitor of the proteasome can be or comprise MG132 and/or bortezomib.

In certain embodiments, a modulator of MDSC/neutrophil recruitment may be or comprise an inhibitor of epidermal growth factor receptor (EGFR). While not being bound by a particular theory, it is thought that CXCL1 and/or CXCL8 can induce EGFR phosphorylation and cellular proliferation, while inhibition of EGFR and/or EGFR kinase can limit CXCL1 and/or CXCL8-induced cell proliferation. In certain embodiments, an inhibitor of EGFR may be or comprise PD153035 and/orAG1478.

In certain embodiments, a modulator of MDSC/neutrophil recruitment can be or comprise an inhibitor of Rho-kinase. While not being bound by a particular theory, it is thought that Rho-kinase inhibition can reduce the formation of CXCL1 and/or CXCL2 and attenuate inflammation. In certain embodiments, a Rho-kinase inhibitor can be or comprise Fasudil and/or Y-27632.

In certain embodiments, a modulator of MDSC/neutrophil recruitment can be or comprise an inhibitor of farnesyltransferase (FTase). While not being bound by a particular theory, it is thought that FTase inhibitors inhibit RET/PTC3-oncogene-induced CXCL1 production. In certain embodiments, FTase inhibitors can be or comprise Chaetomellic acid A, Clavaric acid, FTI-276 trifluoroacetate salt, FTI-277 trifluoroacetate salt, GGTI-297, L-744,832 Dihydrochloride, LNK-754, SCH66336 (Lonafarnib), Manumycin A, R115777 (Zarnestra, Tipifarnib), Gingerol, Gliotoxin, α-hydroxy farnesyl phosphonic acid, or any combination thereof.

In certain embodiments, a modulator of MDSC/neutrophil recruitment can be or comprise an inhibitor of B-cell lymphoma 2 (Bcl-2). While not being bound by a particular theory, inhibition of Bcl-2 is thought to decrease CXCL1 and/or CXCL8 expression and reduces chemokine-associated angiogenesis. In certain embodiments, Bcl-2 inhibitors can be or comprise venetoclax, navitoclax (ABT-263), ABT-199, ABT-737, obatoclax GX-15-070, BL-193, TW37, or any combination thereof.

In certain embodiments, a modulator of MDSC/neutrophil recruitment can be or comprise an inhibitor of P2 nucleotide receptors. While not being bound by a particular theory, inhibition of P2 nucleotide receptors is thought to abrogate neutrophil migration via inhibition of CXCL1. In certain embodiments, an inhibitor of P2 nucleotide receptors can be or comprise: clopidogrel, prasugrel, ticlopidine, ticagrelor, PPADS, or any combination thereof.

In certain embodiments, a modulator of MDSC/neutrophil recruitment can be or comprise an inhibitor of Translocator protein (TSPO). While not being bound by a particular theory, it is thought that agonism of TSPO may inhibit CXCL1 production. In certain embodiments, an agonist of TSPO can be or comprise Ro5-4864.

In certain embodiments, a modulator of MDSC/neutrophil recruitment can be or comprise a microRNA which acts to inhibit and/or antagonize CXCL1 expression and/or signaling. In some embodiments, a microRNA based inhibitor of CXCL1 can be or comprise miR-146a and/or MiR181b.

In certain embodiments, a modulator of MDSC/neutrophil recruitment can be or comprise an inhibitor of AMP-activated protein kinase (AMPK) and/or dachshund family transcription factor 1 (DACH1) signaling. While not being bound by a particular theory, it is thought that disruption of AMPK-DACH1 signaling and/or expression can reduce CXCL1 production. In certain embodiments, an inhibitor of AMPK-DACH1 signaling can be metformin and/or derivatives or variants thereof.

In certain embodiments, a modulator of MDSC/neutrophil recruitment can be or comprise an inhibitor of AMP-activated protein kinase (AMPK). While not being bound by a particular theory, it is thought that AMPK activation can inhibit CXCL8 secretion from cancer cell lines and decrease migration of cancer cells. In certain embodiments, an activator of AMPK can be or comprise AICAR.

In certain embodiments, a modulator of MDSC/neutrophil recruitment can be or comprise an inhibitor of CXCL1 that acts through an as of yet un-defined mechanism. In certain embodiments, an inhibitor of MDSC/neutrophil recruitment can be or comprise Hange-shashin-to (HST), Dexmedetomidine, IMT504 Oligonucleotide, Hes1 transcriptional repressor, Ciglitazone, Fudosteine, Reynosin, Curcumin, DK-139 synthetic chalcone, Angiotensinogen-antisense oligonucleotide, Annexin A1 ligand of formyl peptide receptor 2, dexamethasone corticosteroid, and any combination thereof.

In certain embodiments, a modulator of MDSC/neutrophil recruitment can be or comprise an inhibitor of CXCL2 mediated signaling pathways. In certain embodiments, an inhibitor of CXCL2 mediated signaling pathways is or comprises: a small molecule, an oligonucleotide, a polypeptide and/or a protein. In certain embodiments, an inhibitor of CXCL2 can be or comprise an anti-CXCL2 neutralizing antibody.

In certain embodiments, a modulator of MDSC/neutrophil recruitment may be or comprise an inhibitor of protein kinase B (AKT/PKB). While not being bound by a particular theory, it is thought that disruption of AKT signaling can reduce CXCL2 and/or CXCL8 promoter activity. In certain embodiments, an AKT inhibitor may be or comprise MK2206.

In certain embodiments, a modulator of MDSC/neutrophil recruitment may be or comprise an inhibitor of Mitogen- and stress-activated kinase 1 (MSK1). While not being bound by a particular theory, it is thought that inhibition of MSK1 can enhance CXCL2-included neutrophil adhesion, slow neutrophil migration, and/or potentially inhibit CXCL3 expression. In certain embodiments, an MSK1 inhibitor may be or comprise SB-747651A and/or H89.

In certain embodiments, a modulator of MDSC/neutrophil recruitment may be or comprise an inhibitor of signal transducer and activator of transcription 3 (STAT3) and/or STAT3 mediated signaling pathways. While not being bound by a particular theory, it is thought that STAT3 signaling can promote the expression of inflammatory genes such as CXCL1, CXCL2, and/or CXCL8. In certain embodiments, a STAT3 signaling pathway inhibitor may be or comprise Cryptotanshinone, Capsaicin, Curcumin, Cucurbitacin 1, Celastrol, Atriprimod, PD153035, OleanolicAcid, BrevilinA, Tofacitinib (CP-690,550), Sorafenib, AZD1480, Atiprimod, Auranofin, Sanguinarine, Cucurbitacin 1 (JSI-124), Cucurbitacins B, Cucurbitacin E, Celastrol, Emodin, Dasatinib, Caffeic Acid, CADPE, AG490, WP1066, TG101209, FLL32, Avicin D, E738, MLS-2384, CYT387 (Momelotimib), Ergosterol peroxide, PP2, Ponatinib, Benzyl isothiocyanate, CNTO-328 (Siltuximab), Toclizimab, Cetuximab, KDI1/KDI3/KDI4, Xanthohumol, PY*LKTK(-mts), PY*L, ISS610, PDP/Phosphododeca peptide (-mts), Ac-Y*LPQTV, Hydrocinnamoyl-Tyr(P03H2)-L-cis-3,4-methanoPQ-NHBn, CJ-1383, PM-73G, APT_{STAT3}-9R, Recombinant STAT3 inhibitory peptide aptamer (rS3-PA), DD1/DD2/DD3, Dipicolylamine copper complexes 1,2,3, S31-M2001, STA-21, LL-3, LL-12, Stattic, S31-201/NSC 74859, S31-201.1066/SF-1006, BP-1-102/17o, SH4-54, SH5-07, S31-V3-31/32/33/34, C188, C188-9, Cryptotanshinone, STX-0119, C48, Piperlongumine, OPB-31121, Withacnistin, XZH-5, T2-T3-Celecoxib, HJC0123, Ly5, T40214/T40231, Decoy ODN C*A*T*TTCCCGTTA*A*T*C (* denotes phosphorothioated sites), 13410/13410A/SeqD, CPA-7, IS3295, inS3-54, inS3-54A18, HO-3867, Galiellalactone, BDB-1/BDB-1-9R, Hel2k-Pen/ST3-HA2A, AdCN305-cppSOCS3, Calyculin A, SC-1/SC-43/SC-49, TPA, PF4 (platelet factor 4), Anti-sense AZD9150 (70, NCT01839604), CTLA4apt-STAT3 siRNA, Capsaicin N-vanilyl-8-methyl-1-nonenamide, ML116, derivatives or functional portions thereof, or any combination thereof.

In certain embodiments, a modulator of MDSC/neutrophil recruitment can be or comprise an inhibitor of Geranylgeranyltransferase (GGTase-1). While not being bound by a particular theory, it is thought that inhibition of GGTase-1 may reduce CXCL2 levels. In certain embodiments, a GGTase-1 inhibitor can be or comprise GGTI-2133.

In certain embodiments, a modulator of MDSC/neutrophil recruitment may be or comprise an inhibitor of PI3K-7. While not being bound by a particular theory, it is thought that inhibition of PI3K-7 can reduce CXCL2 expression. In certain embodiments, an inhibitor of PI3K-7 may be or comprise AS252424 and/or IPI-549.

In certain embodiments, a modulator of MDSC/neutrophil recruitment may be or comprise an inhibitor of PI3K/AKT. While not being bound by a particular theory, it is thought that PI3K/AKT signaling can promote CXCL1 and/or CXCL2 secretion. In certain embodiments, a PI3K/AKT inhibitor may be or comprise LY294002.

In certain embodiments, a modulator of MDSC/neutrophil recruitment may be or comprise an inhibitor of pan-PI3K signaling pathways. While not being bound by a particular theory, it is thought that PI3K signaling can promote CXCL8 release and subsequent proliferation and angiogenesis. In certain embodiments, a PI3K signaling pathway inhibitor may be or comprise GDC-0941, Wortmannin, 3-MA 3 methyladenine (3-MA), or any combination thereof.

In certain embodiments, a modulator of MDSC/neutrophil recruitment can be or comprise an inhibitor of activated T cells (NFAT). While not being bound by a particular theory, it is thought that inhibition of NFAT can reduce taurocholate-induced CXCL2 increases and/or reduce CXCL5 expression, and/or potentially attenuate immune system induced tissue damage. In some embodiments, an NFAT inhibitor can be or comprise A-285222.

In certain embodiments, a modulator of MDSC/neutrophil recruitment can be or comprise a microRNA that inhibits and/or antagonizes expression and/or signaling of a chemokine, e.g., a C-X-C-motif chemokine. In some embodiments, an inhibitor of neutrophil recruitment is a microRNA that inhibits and/or antagonizes CXCL2 expression and/or signaling. In some embodiments, a microRNA based inhibitor of CXCL2 can be or comprise miR-532-5p. In certain embodiments, an inhibitor of MDSC/neutrophil recruitment can be or comprise a microRNA that inhibits and/or antagonizes CXCL3 expression and/or signaling. In some embodiments, a microRNA based inhibitor of CXCL3 can be or comprise miR-155.

In some embodiments, a modulator of MDSC/neutrophil recruitment can be or comprise a promoter, agonist, partial agonist, mimetic, or peptide comprising Antithrombin III. While not being limited by a particular theory, it is thought that Antithrombin III can reduce neutrophil recruitment in an anti-inflammatory manner. In certain embodiments, a modulator of MDSC/neutrophil recruitment can be or comprise Thrombate and/or Antithrombin III.

In certain embodiments, a modulator of MDSC/neutrophil recruitment can be or comprise an inhibitor of sphingosine 1-phosphate receptors (S1PR). While not being bound by a particular theory, it is thought that S1PR can promote CXCL5 expression. In certain embodiments, an inhibitor of SIPR can be or comprise FTY720.

In certain embodiments, a modulator of MDSC/neutrophil recruitment may be or comprise an inhibitor of Raf kinase family proteins. While not being bound by a particular theory, it is thought that Raf kinases can facilitate CXCL8 expression in certain cancers and promote cell growth and angiogenesis. In certain embodiments, an inhibitor of Raf kinases may be or comprise Sorafenib/Nexavar (BAY-43-9006), AZ628, PLX4032, Raf265, ZM336372, MCP110, LBT613, ISIS 5132, LErafAON, or any combination thereof, See e.g., Khazak et al., “Selective Raf Inhibition in Cancer Therapy” Expert Opin Ther Targets (2007) 11(12): 1587-1609; the contents of which are incorporated herein in their entirety by reference for the purposes described herein.

In certain embodiments, a modulator of MDSC/neutrophil recruitment can be or comprise an inhibitor of Rho Associated Coiled-Coil Containing Protein Kinase 2 (ROCK2). While not being bound by a particular theory, it is thought that ROCK2 can facilitate NF-κB induced CXCL8 production. In certain embodiments, an inhibitor of ROCK2 can be or comprise Y-27632, KD025, RXC007, or any combination thereof.

In certain embodiments, a modulator of MDSC/neutrophil recruitment may be or comprise an inhibitor of extracellular signal-regulated kinase (ERK)₁ and/or 2. While not being bound by a particular theory, it is thought that ERK signaling promotes cancer proliferation in a manner facilitated by CXCL signaling. In certain embodiments, an ERK1/2 inhibitor may be or comprise PD98059 and/or U0126.

In certain embodiments, a modulator of MDSC/neutrophil recruitment may be or comprise an inhibitor of mechanistic target of rapamycin kinase (mTOR). While not being bound by a particular theory, it is thought that mTOR promotes phosphorylation of p38, ERK1/2, and NF-κB, all of which contribute to CXCL8 expression. In certain embodiments, an inhibitor of mTOR may be or comprise Rapamycin and/or temsirolimus.

In certain embodiments, a modulator of MDSC/neutrophil recruitment can be or comprise an inhibitor of ras homolog family member A (RHOA), cell division cycle 42 (CDC42), and/or rac family small GTPase 1 (RAC) signaling pathways. While not being bound by a particular theory, it is thought that RHOA, CDC42, and RAC signaling facilitates NF-κB phosphorylation and CXCL8 synthesis. In certain embodiments, an inhibitor of RHOA, CDC42, and/or RAC signaling can be or comprise TcdB-10463.

In certain embodiments, a modulator of MDSC/neutrophil recruitment can be or comprise an inhibitor of Src family tyrosine kinase facilitated signaling. While not being bound by theory, it is thought that Src kinases facilitate CXCL8/CXCR-2 mediated MDSC/neutrophil chemotaxis. In certain embodiments, a Scr kinase inhibitor can be or comprise an inhibitor of non-receptor protein tyrosine kinases (e.g., PP1 and/or PP2) and/or SU6656.

In certain embodiments, a modulator of MDSC/neutrophil recruitment can be or comprise an inhibitor of CXCL8, and is a neutralizing antibody or functional portion thereof. In certain embodiments, a CXCL8 neutralizing antibody can be or comprise ABX-IL8, HuMab 10F8, and/or Humax IL8. In certain embodiments, a modulator of MDSC/neutrophil recruitment can be or comprise a microRNA which acts to inhibit and/or antagonize CXCL8 expression and/or signaling. In some embodiments, a microRNA based inhibitor of CXCL8 can be or comprise miR-146a, miR-708, and/or miR-140-3p. In certain embodiments, modulator of MDSC/neutrophil recruitment can be or comprise an inhibitor of CXCL8. In certain embodiments, an inhibitor of CXCL8 can be or comprise IFN-7 Dimeric soluble cytokine, Bisphenol A (BPA), Piperine, certain NSAIDS, TSG-6 secreted glycoprotein, Luteolin natural flavone, SiP serum-borne bioactive lipid T cells, Estradiol Estrogen steroid hormone, or any combination thereof.

In some embodiments, a modulator of MDSC/neutrophil recruitment may be or comprise an inhibitor of IL-8 and/or CXCR1/2 signaling pathway; see e.g., Zarbock et al., “Therapeutic inhibition of CXCR2 by Reparixin attenuates acute lung injury in mice” British Journal of Pharmacology (2008): 155(3): 357-364; the contents of which are incorporated herein in their entirety by reference for the purposes described herein. In some embodiments, an inhibitor of IL-8 and/or CXCR1/2 signaling pathways may be or comprise Ladarixin (LDX), SX-682, reparixin, AZD-8309, or any combination thereof.

In some embodiments, a modulator of MDSC/neutrophil recruitment may be or comprise a modulator of the LTB4 associated signaling pathway. In some embodiments, modulators of the LTB4 associated signaling pathway may comprise specialized pro-resolving mediators such as but not limited to: lipoxins (LXA4), resolvins, protectins and/or maresins.

In some embodiments, a modulator of MDSC/neutrophil recruitment can be or comprise an inhibitor of purinergic receptor P2X4 (P2RX4). While not being bound by a particular theory, it is thought that P2RX4 can promote neutrophil recruitment. In some embodiments, a P2RX4 inhibitor can be or comprise indophagolin, 5-BDBD, BAY-1797, BX430, CTP, NP-1815-PX, PSB-12054, PSB-12062, or any combination thereof.

In some embodiments, a modulator of MDSC/neutrophil recruitment may be or comprise an inhibitor of interleukin 1α (IL-1α) signaling. In some embodiments, such inhibitors may be directed to IL-1α. In some embodiments, such inhibitors may be directed to interleukin-1 receptor, type 1 (IL-1R1) and/or interleukin-1 receptor accessory protein (IL-1R3). Without being bound by a particular theory, it is thought that IL-1α signaling promotes neutrophil recruitment; see e.g., Lee et al. “IL-1α modulates neutrophil recruitment in chronic inflammation induced by hydrocarbon oil” The Journal of Immunology (2011) 186:1747-1754; and Paolo and Shayakhmetov et al. “Interleukin 1α and the inflammatory process” Nature Immunology (2016) 17(8):906-913, the entire contents of each of which are incorporated herein by reference for purposes described herein. In some embodiments, an inhibitor of IL-1α signaling may be or comprise an anti-IL-1α antibody, an anti-IL-1R1 antibody, an anti-IL-1R3 antibody, or any combination thereof.

In some embodiments, a modulator of MDSCs/neutrophils may be or comprise a modulator that decreases survival and/or promotes depletion of MDSC/neutrophils. For example, in some embodiments an inhibitor of MDSC/neutrophil survival, and/or a stimulator (e.g., an agonist) of MDSC/neutrophil depletion comprises an inhibitor of an inhibitor of apoptosis (IAP) family members. While not being bound by theory, it is thought that IAP can inhibit the apoptosis of MDSC/neutrophils; see e.g., Hasegawa et al., “Expression of the inhibitor of apoptosis (IAP) family members in human neutrophils: up-regulation of cIAP2 by granulocyte colony-stimulating factor and overexpression of cIAP2 in chronic neutrophilic leukemia” Blood (2003) 101 (3): 1164-1171; the contents of which are incorporated herein in their entirety by reference for the purposes described herein. In some embodiments, an inhibitor of IAP may be or comprise LCL161, SM-164, SM-406, GDC-0152, ASTX660, AZD5582, birinapant, or any combination thereof.

In some embodiments, an inhibitor of MDSC/neutrophil survival, and/or a stimulator of MDSC/neutrophil depletion can be or comprise an inhibitor of Bruton's tyrosine kinase (BTK). It is thought that BTK influences neutrophil development and function, and that inhibition of BTK may lead to decreased neutrophil counts; see e.g., Fiedler et al., “Neutrophil development and function critically depend on Bruton Tyrosine Kinase in a mouse model of X-linked agammaglobulinemia” Blood (2011); 117(4): 1329-39; the contents of which are incorporated herein in their entirety by reference for the purposes described herein. In some embodiments, an inhibitor of BTK can be or comprise ibrutinib, spebrutinib, branebrutinib, fenebrutinib, evobrutinib, CNX-774, PCI 29732, zanubrutinib, or any combination thereof.

In some embodiments, an inhibitor of MDSC/neutrophil survival, and/or a stimulator of MDSC/neutrophil depletion can be or comprise an inhibitor of tyrosine kinases. It is thought that tyrosine kinases such as BCR/abl, Src, c-Kit, and/or ephrin receptors may function to inhibit the proinflammatory functions of mature human neutrophils; see e.g., Futosi et al., “Dasatinib inhibits proinflammatory functions of mature neutrophils” Blood (2012); 119(21): 4981-4991; the contents of which are incorporated herein in their entirety by reference for the purposes described herein. In some embodiments, an inhibitor of tyrosine kinases can be or comprise dasatinib.

In some embodiments, an inhibitor of MDSC/neutrophil survival, and/or a stimulator of MDSC/neutrophil depletion may be or comprise an agonist and/or activator of nucleotide binding oligomerization domain containing -1 and/or -2 (NOD1/2). It is thought that neutrophils express NOD-like receptors (NLRs), and that NOD1 signaling regulates the migration and phagocytic capacity of neutrophils, wherein its ligation leads to the activation of NFxB and MAPKs in neutrophils; see e.g., Ekman and Cardell “The expression and function of Nod-like receptors in neutrophils” Immunology (2010) 130(1): 55-63; Jeong et al., “Nod2 and Rip2 contribute to innate immune responses in mouse neutrophils” Immunology (2014) 143(2): 269-276; and Ajendra et al., “NOD2 dependent neutrophil recruitment is required for early protective immune responses against infectious Litomosoides sigmodontis L3 larvae” Scientific Reports (2016) 6, 39648; the contents of each of which are incorporated herein in their entirety by reference for the purposes described herein. In certain embodiments, an agonist of NOD1/2 may be or comprise M-TriDAP [N-acetyl-muramyl-L-Ala-γ-D-Glu-meso-diaminopimelic acid], DAP and derivatives (e.g., iE-DAP), including acylated derivatives (e.g., C12-iE-DAP), MDP [N-Acetylmuramyl-L-Alanyl-D-Isoglutamine, aka MurNAc-L-Ala-D-isoGln, aka muramyl dipeptide] and derivatives, including acylated derivatives (e.g., L18-MDP), N-glyscosylated MDP, Murabutide, M-TriLYS, or any combination thereof. In some embodiments, such an agonist may be administered in an amount that is effective to inhibit neutrophil recruitment and/or survival.

In some embodiments, an inhibitor of MDSC/neutrophil survival, and/or a stimulator of MDSC/neutrophil depletion may be or comprise an agonist of TNF-Related Apoptosis-Inducing Ligand Receptor (TRAIL-R) signaling. While not being bound by a particular theory, it is thought that stimulating TRAIL-R signaling may trigger MDSC/neutrophil apoptosis and clearance from tissues. In certain embodiments, TRAIL-R agonists may be or comprise Mapatumumab, AMG 951, TRM-1, or any combination thereof.

In some embodiments, an inhibitor of MDSC/neutrophil survival, and/or a stimulator of MDSC/neutrophil depletion may be or comprise an inhibitor of a dopaminergic receptor and/or an antipsychotic agent. In some embodiments, such inhibitors may be directed to a dopamine receptor D2. While not being bound by a particular theory, it is thought that inhibitors of dopaminergic receptors and/or antipsychotic agents reduce neutrophil survival and/or promote neutrophil depletion; see e.g., Compazine (prescribing information), Research Triangle Park, NC: GlaxoSmithKline; July 2004; the contents of which are incorporated herein in their entirety by reference for the purposes described herein. In some embodiments, an inhibitor of a dopaminergic receptor and/or an antipsychotic agent may be or comprise a butyrophenone (e.g., benperidol, bromperidol, droperidol, haloperidol, moperone, pipamperone, timiperone, melperone, and lumateperone), a diphenylbutylpiperidine (e.g., flusirilene, penfluridol, and pimozide), a phenothiazine (e.g., acepromazine, chlorpromazine, cyamemazine, dixyrazine, fluphenazine, levomepromazine, mesoridazine, perazine, pericyazine, perphenazine, pipotiazine, prochlorperazine, promaizne, promethazine, prothipendyl, tioproperazine, thioridazine, trifluoperzine, and triflupromazine), a thioxanthene (e.g., chlorprothixene, clopenthixol, flupentixol, thiothixene, and zuclopenthixol), a benzamide (e.g., sulpiride, sultopride, veralipride, amisulpride, nemonapride, remoxipride, and sultopride), a tricyclic (e.g., carpipramine, clocapramine, clorotepine, clotiapine, loxapine, mosapramine, asenapine, clozapine, olanzapine, quetiapine, and zotepine), a benzisoxazole/benzisothiazole (e.g., iloperidone, lurasidone, paliperidone, paliperidone palmitate, perospirone, risperidone, and ziprasidone), a phenylpiperazine/quinolinone (e.g., aripiprazole, aripiprazole lauroxil, brexpiprazole, and cariprazine), other agents (e.g., blonanserin, pimavanserin, and sertindole), or combinations thereof.

In some embodiments, an inhibitor of MDSC/neutrophil survival, and/or a stimulator of MDSC/neutrophil depletion may be or comprise an agent that causes neutropenia. In some embodiments an agent that causes neutropenia may be or comprise abacavir, acetaminophen, acetosulfone, acitretin, ajmaline, allopurinol, aminoglutethimide, aminopyrine, amodiaquine, amoxapine, alkylating agents, amoxicillin, ampicillin, amygdalin, aprindine, angiotensin converting enzyme (ACE) inhibitors, anthracyclines, antiarrhythmic agents, antimetabolites, benoxaprofen, bepridil, bezafibrate, bucillamine, benzylpenicillin, calcium dobesilate, captopril, carbenecillin, camptothecins, carbamazepine, carbimazole, cefamandole, cefipime, ceftriaxone, cefotaxime, cefuroxime, cephalexin, cephalotihn, cephapirin, caphazolin, cephradine, chloramphenicol, chloroguanide, chlorpheniramine, chlorpromazine, chlorpropamide, chlorthalidone, cimetidine, clarithromycin, clomipramine, clopidogrel, cloxacillin, ciprofloxacin, clindamycin, clozapine, cotrimoxazole, cyanamide, dapsone, deferiprone, desipramine, diclofenac, diflunisal, dipyrone, disopyramide, dothiepin, doxepin, doxycycline, enalapril, erythromycin, epipodophyllotoxins, famotidine, fenbufen, fenoprofen, fluconazole, flucytosin, fluoxetine, flutamide, fusidic acid, gentamicin, gold, H2 blockers, hydroxychloroquine, hydroxyurea, ibuprofen, infliximab, imatinib, imipenem/cilastatin, imipramine, indalpine, indinavir, infliximab, interferon alpha, interleukin 12, isoniazid, isothretinoin, lamotrigine, levamisole, levetiracetam, linezolid, lincomycin, maprotiline, mebendazole, mebhydrolin, meclofenamic acid, mefenamic acid, mefloquine, meprobamate, mesalazine, methaqualone, methazolamide, methotrimeprazine, methyldopa, metiamide, metoclopramide, metolazone, mezlocillin, mianserin, minocycline, moxalactam, meropenem, metamizole, methimazole, mitomycin C, metronidazole, nafamostat, nafcillin, naproxen, nifedipine, nifuroxazide, nilutamide, nitrofurantoin, norfloxacin, olanzapine, omeprazole, oxacillin, non-steroidal anti-inflammatory drugs (NSAIDs), noramidopyrine, olanzapine, oxacillin, penicillamine, pentamidine, pentazocine, pentobarbital, perazine, phenindione, phenylbutazone, phenytoin, penicillin G, piperacillin-tazobactam, procainamide, propylthiouracil, pirenzepine, piroxicam, povidone iodine, prednisone, promethazine, propafenone, propranolol, propylthiouracil, pyrazolone derivatives, pyrithioxine, pyrithyldione, asquetiapine, quinidine/quinine, Ramipril, ranitidine, rifabutin, riluzole, risperidone, riodrine, roxithromycin, rituximab, salazopyrine, sulfasalazine, sertraline, spironolactone, sulfaguanidine, sulindac, suramin, tamoxifen, terbinafine, thiopronine, tacrolimus, taxanes, teicoplanin, thiamazole, thioridazine, thiothixene, ticarcillin, tocainide, tolbutamide, tolmetin, trazodone, trimethoprim, thionamides, ticlopidine, trimethoprim/sulfamethoxazole, tobramycin, torsemide, valproic acid, vancomycin, vesnarinone, valganciclovir, venlafaxine, vinblastineyohimbine, zidovudine, ziprasidone, zomepirac, or any combination thereof, see e.g., Moore “Drug-induced neutropenia” P&T (2016) 41(12):765-768; Curtis “Non-chemotherapy drug-induced neutropenia: key points to manage the challenges” Hematology The American Society of Hematology Education Program (2017) 2017(1):187-193; and http://adverse-effects.com/documents/case_reports_agranulocytosis.pdf; the contents of which are incorporated herein by reference for the purposes described herein.

In some embodiments, more than one modulator of MDSC/neutrophils (e.g., described herein) may be included in compositions described herein. In some embodiments, a modulator of MDSC/neutrophil (e.g., described herein) may be used in combination with other therapeutic agents.

In some embodiments, modulators described herein are administered in an amount that is effective to inhibit MDSC/neutrophil recruitment and/or survival. Therefore, in some embodiments, modulators described herein may be administered in an amount that is higher than what is typically used in other therapeutic context. In some embodiments, modulators described herein may be administered in an amount that is lower than what is typically used in other therapeutic context.

B) Modulating MDSC/Neutrophil Effector Function

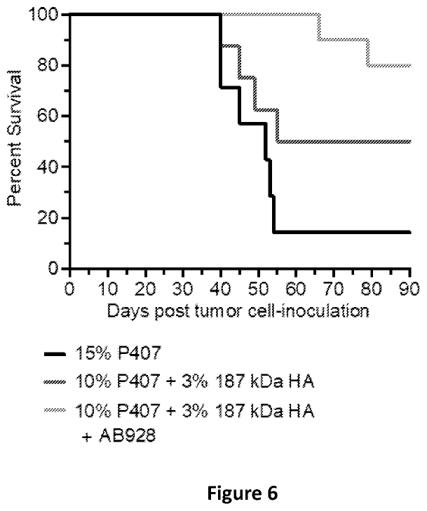

In some embodiments, a composition described herein comprises a biomaterial (e.g., polymeric biomaterial) and a modulator of MDSCs, and more particularly, a modulator of neutrophils, that modulates their effector function. In some embodiments, such a modulator of neutrophils and/or MDSCs may modulate production and/or secretion of immunomodulatory factors (e.g., such as the cytokines and chemokines described above) by neutrophils and/or MDSCs, which in some embodiments may promote recruitment and/or survival of cancer cells and/or other immunostimulatory cell types (e.g., NK cells, T cells, γδ T cells, dendritic cells, neutrophils and/or macrophages (see e.g., Benigni et al., “CXCR3/CXCL10 Axis Regulates Neutrophil-NK Cell Cross-Talk Determining the Severity of Experimental Osteoarthritis” The Journal of Immunology, (2017); Minns et al., “Orchestration of Adaptive T Cell Response by Neutrophil Granule Contents” Mediators of Inflammation (2019); Leleifeld et al., “How neutrophils shape adaptive immune responses” Frontiers in Immunology (2015); Li et al., “The regulatory roles of neutrophils in adaptive immunity” Cell Communication and Signaling (2019); and Laban “Vasodilator-stimulated phosphoprotein regulates leukocyte infiltration, polarization and metabolism during vascular repair in the ischemic hindlimb” Thesis—Goethe-Universität Frankfurt am Main (2018) FIG. 6; the contents of each of which are incorporated herein in their entirety by reference for the purposes described herein), and/or in some embodiments may promote depletion of other immunosuppressive cell types. In some embodiments, such a modulator of neutrophils and/or MDSCs may promote induction of neutrophils and/or MDSCs to anti-tumor phenotype. In some embodiments, such a modulator of neutrophils and/or MDSCs may modulate extracellular matrix modifying capabilities of neutrophils and/or MDSCs.

In some embodiments, a modulator of MDSC/neutrophil effector function may be or comprise one or more modulators of MDSC/neutrophils that act to inhibit recruitment and/or simulate depletion of MDSC/neutrophils described herein. For example, in some embodiments, a modulator of MDSC/neutrophil effector function is an inhibitor of one or more neutrophil-derived chemokines, such as the C-C motif chemokine signaling pathways and/or C-X-C motif signaling pathways as described herein. In some embodiments, a modulator of MDSC/neutrophil effector function may be or comprise an anti-CD47 antibody, an anti-CSF1 antibody, an anti-CSF1R antibody, or any combination thereof. In certain embodiments, a modulator of MDSC/neutrophil effector function may be or comprise SRF231, Hu5F9-G4, CC-900002, TTI-621 (anti-CD47 antibodies), or any combination thereof. In certain embodiments, a modulator of MDSC/neutrophil effector function may be MCS-110 (an anti-CSF1 antibody). In certain embodiments, a modulator of neutrophil effector function may be FPA008, RG7155, IMC-CS4, AMG820, UCB6352 (anti-CSF1R antibodies), or any combination thereof. In certain embodiments, a modulator of neutrophil effector function may be a small molecule inhibitor of CSF1R. In certain embodiments, a modulator of neutrophil effector function may be BLZ945, GW2580, PLX3397 (small molecule inhibitors of CSF1R), or any combination thereof. In certain embodiments, a modulator of neutrophil effector function may be or comprise a BTK inhibitor (e.g., zanubrutinib), an ITK inhibitor, a PI3K inhibitor, a PI3K7 inhibitor, a PI3K6 inhibitor, or any combination thereof.

In some embodiments, a modulator of MDSC/neutrophil effector function may be or comprise an inhibitor of a TGFβ signaling pathway. While not being bound by a particular theory, the presence of transforming growth factor-β (TGFβ) has been demonstrated to promote a pro-tumor phenotype (N2-like phenotype); see e.g., Giannelli et al., “Biomarkers and overall survival in patients with advanced hepatocellular carcinoma treated with TGF-βRI inhibitor galunisertib” PLOS One (2020); and Fridlender et al., “Polarization of Tumor-Associated Neutrophil (TAN) Phenotype by TGF-β: “N1” versus “N2” TAN” Cancer Cell (2009) 16(3): 183-194; the contents of each of which are incorporated herein in their entirety by reference for the purposes described herein. In addition, while not being bound by a particular theory, TGFβ is thought to function as a potent MDSC/neutrophil chemoattractant, and in some embodiments a modulator of MDSC/neutrophil recruitment may be or comprise an inhibitor of TGFβ; see e.g., Reibman et al., “Transforming growth factor beta 1, a potent chemoattractant for human neutrophils, bypasses classic signal-transduction pathways” Proc Natl Acad Sci USA (1991) 88(15): 6805-6809; and Brandes et al., “Type I transforming growth factor-beta receptors on neutrophils mediate chemotaxis to transforming growth factor-beta” Journal of Immunology (1991) 147(5): 1600-1606; the contents of each of which are incorporated herein in their entirety by reference for the purposes described herein. In some embodiments, an inhibitor of TGFβ signaling pathway may be or comprise TGFβR1 kinase inhibitors (e.g., galunisertib), anti-TGFβ monoclonal antibodies (e.g., Fresolimumab), TGFβ signaling pathway inhibitors (e.g., vactosertib, RepSox, GW788388, LY364947, SB505124, SB525334, K02288, and/or LDN-193189), or any combination thereof.

In certain embodiments, a modulator of MDSC/neutrophil effector function and/or recruitment may be a modulator of an adenosine metabolism and/or recognition pathway. While not being limited by a particular theory, it is thought that extracellular Adenosine can act via the A₁ and A3 adenosine receptor subtypes to promote neutrophil chemotaxis and phagocytosis, while at higher concentrations, adenosine can act on the lower-affinity A2A and A2B receptors to inhibit neutrophil trafficking and effector functions such as oxidative burst, inflammatory mediator production, and/or granule release; see e.g., Barletta et al., “Regulation of neutrophil function by adenosine” Arterioscler Thromb Vasc Biol (2012) 32(4): 856-864; the contents of which are incorporated herein in their entirety by reference for the purposes described herein. Additionally, while not being limited by a particular theory, it is thought that adenosine receptor antagonists (e.g., theophylline) can reduce neutrophil chemotaxis and induce neutrophil apoptosis; see e.g., Mehta et al. “Theophylline alters neutrophil function in preterm infants” Biology of the Neonate (2002) 81:176-181; and Yasui et al. “Theophylline induces neutrophil apoptosis through adenosine A2A receptor antagonism” Journal of Leukocyte Biology (2000) 67:529-535; the contents of each of which are incorporated herein in their entirety by reference for the purposes described herein. In certain embodiments, an inhibitor of an adenosine associated pathway may be an inhibitor of A2A and/or A2B adenosine receptors. In certain embodiments, an inhibitor of A2A and/or A2B adenosine receptor may be or comprise etrumadenant (AB928), MRS-1754, PSB-0788, CGH-2466, istradefylline, AZD4635, MK-3814, ZM-241385, ANR-94, SCH-442416, SCH-58261, TC-G 1004, 8-(3-chlorostyryl)caffeine, CPI-444, PBF-509, alloxazine, PSB-1115, PSB-603, GS-6201, caffeine, BAY-545, theophylline, or any combination thereof, see e.g., Leone & Emens “Targeting adenosine for cancer immunotherapy” J Immunother Cancer (2018) 6:57; the contents of which are incorporated herein in their entirety by reference for the purposes described herein.

In certain embodiments, an inhibitor of an adenosine associated pathway may be an inhibitor of CD39 and/or CD73. In certain embodiments, an inhibitor of CD39 and/or CD73 signaling pathways may be or comprise an anti-CD39 antibody, an anti-CD73 antibody, POM1, IPH52, AB680, BMS-986179, MEDI9447, PSB-12379, CD73-IN-1, MethADP, or any combination thereof.

In certain embodiments, an inhibitor of an adenosine associated pathway can be a modulator (e.g., an agonist or an antagonist) of purinergic receptor P2X7 (P2RX7). In certain embodiments, an inhibitor of P2RX7 can be or comprise GSK1482160, JNJ-5417544, JNJ-479655, CE-224535, A-804598, Brilliant Blue G (BBG), AZD9056, KN-62, AZ-11645373, AZ-10606120, GW791343, GSK314181A, AFC-5128, EVT-401, or combinations thereof, see e.g., Savio et al., “The P2X7 Receptor in Inflammatory Diseases: Angel or Demon?” Frontiers in Pharmacology, (2018); the contents of which are incorporated herein in their entirety by reference for the purposes described herein. In certain embodiments, agonists of P2RX7 may comprise but are not limited to BzATP.

In certain embodiments, a modulator of MDSC/neutrophil effector function can be or comprise an inhibitor of ataxia-telangiectasia mutated (ATM) kinase. While not being bound by a particular theory, it is thought that inhibition of ATM kinase can reduce CXCL1 conferred tumor radioresistance; see e.g., Zhang et al., “CAF-secreted CXCL1 conferred radioresistance by regulating DNA damage response in a ROS-dependent manner in esophageal squamous cell carcinoma” Cell Death Disc. (2017) 8:e2790; the contents of which are incorporated herein in their entirety by reference for the purposes described herein. In certain embodiments, an inhibitor of ATM kinase can be or comprise Ku55933.

In certain embodiments, a modulator of MDSC/neutrophil effector function can be or comprise an inhibitor of adenosine deaminase acting on RNA -1 (ADAR1). While not being limited by a particular theory, it is thought that ADAR1 enzymatic activity edits interferon-inducible RNA species, reducing substrates for protein kinase R (PKR) and melanoma differentiation-associated protein 5 (MDA5) innate immune activity; see e.g., Ishizuka et al., “Loss of ADAR1 in tumours overcomes resistance to immune checkpoint blockade” Nature (2019) 565, 43-48; the contents of which are incorporated herein in their entirety by reference for the purposes described herein. In some embodiments, a modulator of MDSC/neutrophil recruitment is or comprises an inhibitor of ADAR1. In some embodiments, an inhibitor of ADAR1 activity can be or comprise 8-azaadenosine. In some embodiments, an inhibitor of ADAR1 expression can be or comprise an inhibitor of enhancer of zeste homolog 2 (EZH2) (e.g., GSK126).

In certain embodiments, a modulator of MDSC/neutrophil effector function may be or comprise an inhibitor of a phosphoinositide 3-kinase (PI3K)-associated pathway. While not being limited by a particular theory, it is thought that a PI3K pathway may promote MDSC/neutrophil-mediated inhibition of T cells. In some embodiments, a modulator of MDSC/neutrophil recruitment may be or comprise an inhibitor of PI3K. In some embodiments, an inhibitor of PI3K signaling may be or comprise Buparlisib.

In some embodiments, a modulator of MDSC/neutrophil effector function may be or comprise an inhibitor of a COX1 and/or COX2 mediated signaling pathway. While not being bound by a particular theory, PGE2 (a terminal prostaglandin in the COX pathway) is thought to promote anti-inflammatory neutrophil phenotypes at a site of injury and/or modulate inflammation in-vivo; see e.g., Loynes et al., “PGE2 production at sites of tissue injury promotes an anti-inflammatory neutrophil phenotype and determines the outcome of inflammation resolution in-vivo” Science Advances (2018): Vol. 4, no. 9, eaar8320; and Turcotte et al., “The Endocannabinoid Metabolite Prostaglandin E 2 (PGE2)-Glycerol Inhibits Human Neutrophil Functions: Involvement of Its Hydrolysis into PGE2 and EP Receptors” Journal Immunology (2017); 198:3255-3263; the contents of each of which are incorporated herein in their entirety by reference for the purposes described herein. In some embodiments, PGE2 is thought to function as an inhibitor of certain proinflammatory neutrophil functions, such as leukotriene B4 (LTB4) biosynthesis, reactive oxygen species (ROS) production, and/or neutrophil migration. In certain embodiments, a COX1 and/or COX2 inhibitor may be or comprise, but not limited to: (i) salicylates (e.g., acetylsalicylic acid, diflunisal, salicylic acid and other salicylates, and/or salsalate); (ii) propionic acid derivatives (e.g., ibuprofen, carprofen, dexiburofen, naproxen, fenoprofen, ketoprofen, dexketoprofen, flurbiprofen, oxaprozin, and/or loxoprofen; (iii) acetic acid derivatives (e.g., indomethacin, tolmetin, sulindac, etodolac, ketorolac (e.g., a salt of ketorolac including, e.g., but not limited to ketorolac tromethamine), diclofenac, aceclofenac, amfenac, and/or nabumetone); (iv) enolic acid (oxicam) derivatives (e.g., piroxicam, meloxicam, tenoxicam, droxicam, lornoxicam, isoxicam, and/or phenylbutazone); (v) anthranilic acid derivatives or fenamates (e.g., mefenamic acid, meclofenamic acid, flufenamic acid, and/or tolfenamic acid); (vi) selective COX-2 inhibitors (e.g., celecoxib, rofecoxib, valdecoxib, parecoxib, lumiracoxib, etoricoxib, and/or firocoxib); (vii) sulfonanilides (e.g., nimesulide); (viii) others (e.g., clonixin, SC-560, TFAP, licofelone [e.g., acts by inhibiting lipoxygenase (LOX) and COX], H-harpagide), or combinations thereof.

In some embodiments, a modulator of MDSC/neutrophil effector function may be or comprise a promoter, agonist, partial agonist, mimetic, or peptide comprising a specialized pro-resolving mediators (SPMs) (e.g., such as arachidonic acid (AA)-derived lipoxins and docosahexaenoic acid (DHA)-derived resolvins such as resolvinD2 (RvD2) and/or LXA4). While not being bound by a particular theory, SPMs are long-chain fatty acid-derived lipid mediators, which are involved in a coordinated resolution program to prevent excessive inflammation and/or to resolve acute inflammatory response. While not being bound by a particular theory, resolvin D2 (RvD2) is thought to restore neutrophil directionality, limit neutrophil infiltration, and/or mediate protection from neutrophil-initiated second-organ injury; see e.g., Kurihara et al., “Resolvin D2 restores neutrophil directionality and improves survival after burns” FASEB Journal (2013): 27(6): 2270-2281; and Serhan & Levy “Resolvins in inflammation: emergence of the pro-resolving superfamily of mediators” The Journal of Clin Investigation (2018); Cai et al., “MerTK cleavage limits proresolving mediator biosynthesis and exacerbates tissue inflammation” PNAS, 113: 6526-6531 (2016); Sulciner et al., “Resolvins suppress tumor growth and enhance cancer therapy” J Exp Med 215: 115-140 (2018), and Serhan et al., “Novel anti-inflammatory—Pro-resolving mediators and their receptors” Curr Top Med Chem 11: 629-647 (2011); the contents of each of which are incorporated herein in their entirety by reference for the purposes described herein. In certain embodiments, compositions described herein comprise a resolvin, in some embodiments said resolvin may be but is not limited to: RvD1, RvD2, RvD3, RvD4, RvD5, RvD6, 17R-RvD1, 17R-RvD2, 17R-RvD3, 17R-RvD4, 17R-RvD5, 17R-RvD6, RvE1, 18S-RvE1, RvE2, RvE3, RvT1, RvT2, RvT3, RvT4, RvDln-3, RvD2n-3, RvD5n.3, and/or combinations thereof. In some embodiments, a SPM that may be useful as a modulator of myeloid-derived suppressive cells may be or comprise a lipoxin (including, e.g., LxA4, LxB4, 15-epi-LxA4, and/or 15-epi-LxB4), a protectin/neuroprotectin (e.g., DHA-derived protectins/neuroprotectins and/or n-3 DPA-derived protectins/neuroprotectins), maresins (e.g., DHA-derived maresins and/or n-3 DPA-derived maresins), other DPA metabolites, or any combination thereof.

In some embodiments, a modulator of MDSC/neutrophil effector function may be or comprise an inhibitor of phosphodiesterase-5 (PDE5). While not being limited by a particular theory, it is thought that inhibition of PDE5 may reduce ARG1, NOS2, and/or IL-4Ra expression in N1-like TANs, and/or inhibit PDE5 induced stimulation of neutrophil degranulation; see e.g., Puzzo et al., “Role of phosphodiesterase 5 in synaptic plasticity and memory” Neuropsychiatr Dis Treat (2008): 4(2): 371-387; and Noel et al., “PDE5 inhibitors as potential tools in the treatment of cystic fibrosis” Frontiers in Pharmacology (2012); the contents of each of which are incorporated herein in their entirety by reference for the purposes described herein. In certain embodiments, an inhibitor of PDE5 may be or comprise Sildenafil, Tadalafil, Vardenafil, Udenafil, Avanafil, or any combination thereof.

In some embodiments, a modulator of MDSC/neutrophil effector function can be or comprise metformin (also known as dimethylbiguanide). While not being bound by a particular theory, it is thought that metformin may impair the ability of MDSCs and/or Neutrophils to suppress T cells, reduce intratumoral hypoxia, and/or modulate innate immune-mediated inflammation; see e.g., Oliveira et al., “Metformin modulates innate immune-mediated inflammation and early progression of NAFLD associated hepatocellular carcinoma in zebrafish” Journal of Hepatology (2019), 70, 710-721; Sharping et al., “Efficacy of PD-1 Blockade is Potentiated by Metformin-Induced Reduction of Tumor Hypoxia” Cancer Immunology Research (2017); and Baumann et al., “Regulatory myeloid cells paralyze T cells through cell-cell transfer of the metabolite methylglyoxal” Nature Immunology (2020) 21, 555-566; the contents of each of which are incorporated herein in their entirety by reference for the purposes described herein.

In some embodiments, a modulator of MDSC/neutrophil effector function and/or recruitment can be or comprise a modulator triggering receptor expressed on myeloid cells (TREM) proteins (e.g., TREM-1 and/or TREM-2). Without being bound by any particular theory, it is thought that expression and/or activation (e.g., ligation) of TREM-1 on polymorphonuclear neutrophils regulates innate immune activation in infectious and non-infectious conditions, likely through phosphatidyl-inositol 3 kinase (PI3K) function, where pathway activation can trigger all neutrophil effector functions; see e.g., Fortin et al., “Effects of TREM-1 activation in human neutrophils: activation of signaling pathways, recruitment into lipid rafts and association with TLR4” Int Immunology (2007) 19(1):41-50; and Baruah et al., “TREM-1 regulates neutrophil chemotaxis by promoting NOX-dependent superoxide production” J Leukoc Biol (2019) 105(6)1195-1207; and Alflen et al., “Idelalisib impairs TREM-1 mediated neutrophil inflammatory responses” Scientific reports (2018) 8:5558; the contents of each of which are incorporated herein in their entirety by reference for the purposes described herein. TREM-1 is non-covalently associated with the DNAX activation protein of 12 kDa (DAP12). Phosphorylation of DAP12 leads to binding of the Src homology 2 (SH2) domains to form receptor complexes for further stimulation and amplification of the inflammatory response. While not being limited by a particular theory, it is thought that TREM-1 plays a key role in some diseases, such as inflammatory bowel disease, acute pancreatitis, gouty arthritis, and atherosclerosis; see e.g., Feng et al., “Therapeutic Effect of Modulating TREM-1 via Anti-inflammation and Autophagy in Parkinson's disease” Frontiers in Neuroscience (2019); the contents of which are incorporated herein in their entirety by reference for the purposes described herein. On the other hand, while not being limited by a particular theory, the precise role of TREM-2 is less forthcoming; TREM-2 is thought to participate in inhibition of inflammatory cytokine production during microbial challenge, in certain cancers may function as a tumor suppressor, is thought to function in the remodeling of the tumor associated myeloid cell landscape, and is also thought to be commonly expressed on immunosuppressive MDSC/neutrophils; see e.g., Tang et al., “TREM-2 acts as a tumor suppressor in hepatocellular carcinoma by targeting the PI3K/Akt/3-catenin pathway” Oncogenesis (2019), 8:9; and Molgora et al., “TREM-2 Modulation Remodels the Tumor Myeloid Landscape Enhancing Anti-PD-1 Immunotherapy” Cell (2020); the contents of each of which are incorporated herein in their entirety by reference for the purposes described herein.

In some embodiments, compositions described herein can comprise a TREM-1 inhibitor, wherein the TREM-1 inhibitor can be or comprise an anti-TREM-1 antibody (PY159, Pionyr Immunotherapeutics), TLT-1-CDR2 (SAVDRRAPAGRR), TLT-1—CDR3 (CMVDGARGPQILTHR), LR17 (LQEEDAGEYGCMVDGAR), LR6-1 (LQEEDA), LR6-2 (EDAGEY), LR6-3 (GEYGCM) (e.g., as described in international publication WO2017/007712A1; the contents of which are incorporated herein in their entirety by reference for the purposes described herein), LR12 (LQEEDAGEYGCM) (e.g., as described in Tammaro et al., “TREM-1 and its potential ligands in non-infectious diseases: from biology to clinical perspectives” Pharmacology & Therapeutics (2017), Vol 177, 81-95; the contents of which are incorporated herein in their entirety by reference for the purposes described herein), SCHOOL peptides (e.g., as described in Shen & Sigalov “Novel TREM-1 Inhibitors Attenuate Tumor Growth and Prolong Survival in Experimental Pancreatic Cancer” Mol. Pharmaceutics (2017) 14, 12, 4572-4582; which is incorporated herein by reference for the purpose described herein), LP17 (LQVTDSGLYRCVIYHPP) (e.g., as described in Feng et al., “Therapeutic Effect of Modulating TREM-1 via Anti-inflammation and Autophagy in Parkinson's Disease” Frontiers in Neuroscience (2019); the contents of which are incorporated herein in their entirety by reference for the purposes described herein), GF9 (GFLSKSLVF), GE31, (GFLSKSLVFPYLDDFQKKWQEEM(O)ELYRQKVE), GA31 (GFLSKSLVFPLGEEM(O)RDRARAHVDALRTHLA) (e.g., as described in Tornai et al., “Inhibition of Triggering Receptor Expressed on Myeloid Cells 1 Ameliorates Inflammation and Macrophage and Neutrophil Activation in Alcoholic Liver Disease in Mice” Hepatology Communications (2019) 3(1); the contents of which are incorporated herein in their entirety by reference for the purposes described herein), LSKSLVF (e.g., as described in Gibot et al., “Triggering Receptor Expressed on Myeloid Cells-1 Inhibitor Targeted to Endothelium Decreases Cell Activation” Frontiers in Immunology (2019) 10: 2314; the contents of which are incorporated herein in their entirety by reference for the purposes described herein), M3 (RGFFRGG) (e.g., as described in Denning et al., “Extracellular CIRP as an endogenous TREM-1 ligand to fuel inflammation in sepsis” JCI Insight (2020); the contents of which are incorporated herein in their entirety by reference for the purposes described herein), prodrugs thereof, conjugated versions thereof, deuterated variations thereof, analogs thereof comprising non-naturally occurring amino-acids, functional variations thereof including a different sequence of amino acids but which retain TREM-1 inhibitory activity, analogs thereof in which each amino acid can be, individually, a D or L isomer, and combinations of L-isoforms with D-isoforms thereof, or any combination thereof.

In some embodiments, compositions described herein may comprise a TREM-1 inhibitor, wherein the TREM-1 inhibitor may be or comprise a PI3K signaling pathway inhibitor. In some embodiments, an inhibitor of PI3K signaling may be or comprise dactolisib (BEZ235), pictillisib (GDC-0941), LY294002, idelalisib (CAL-101, GS1101), buparlisib (BKM120), SRX3207, PI-103, NU7441 (KU-57788), TGX-221, IC-87114, wortmannin, XL147 analogue, ZSTK474, alpelisib (BYL719), AS-605240, PIK-75 HCl, rigosertib (ON-01910), 3-Methyladenine (3-MA), A66, voxtalisib (XL765) analogue, omipalisib (GSK2126458), PIK-90, AZD6482, PF-04691502, apitolisib (GDC-0980), GSK1059615, duvelisib (IPI-145), gedatolisib (PKI-587), TG100-115, AS-252424, BGT226 maleate (NVP-BGT226 maleate), fimepinostat (CUDC-907), PIK-294, AS-604850, GSK2636771, copanlisib (BAY 80-6946), CH5132799, CAY10505, PIK-293, PKI-402, TG100713, VS-5584 (SB2343), taselisib (GDC 0032), CZC24832, AMG319, GSK2292767, paxalisib (GDC-0084), MTX-211, seletalisib (UCB-5857), GDC-0326, HS-173, SF2523, leniolisib (CDZ173), serabelisib (TAK-117), IPI-549, Quercetin, bimiralisib (PQR309), VPS34 inhibitor 1 (Compound 19), voxtalisib (XL765), autophinib, GNE-317, notoginsenoside R1, tenalisib (RP6530), umbralisib (TGR-1202), acalisib (GS-9820), nemiralisib (GSK2269557), samotolisib (LY3023414), VPS34-IN1, 2-D08, IPI-3063, SAR405, PIK-III, PI-3065, quercetin dihydrate, pilaralisib (XL147), AZD8835, deguelin, selective PI3K6 inhibitor 1 (compound 7n), PF-4989216, AZD8186, GNE-477, oroxin B, or any combination thereof.

In some embodiments, compositions described herein can comprise a TREM-1 inhibitor, wherein the TREM-1 inhibitor can be selected from the group comprising but not limited to: MicroRNA 294, human cathelicidin LL-37, the F-c portion of human IgG (AdTREM-lIg), antibodies directed to the TREM-1 and/or sTREM-1 or TREM-1 and/or sTREM-1 ligands, and fragments thereof which also inhibit TREM-1, small molecules inhibiting the function, activity or expression of TREM-1, siRNAs directed to TREM-1, shRNAs directed to TREM-1, antisense oligonucleotides directed to TREM-1, ribozymes directed to TREM-1, aptamers which bind to and inhibit TREM-1, fusion proteins between human IgGl constant region and the extracellular domain of mouse TREM-1 or that of human TREM-1 (e.g., as described in international publication WO2017/007712A1; the contents of which are incorporated herein in their entirety by reference for the purposes described herein), and any combination thereof.

While not being bound by a particular theory, it is thought that TREM-2 signals through its association with TYRO protein tyrosine kinase binding protein (TYROBP), also known as DNAX-activating protein of 12 kDa (DAP12), which recruits the spleen tyrosine kinase (SYK) through its cytosolic immunoreceptor tyrosine-based activation motifs (ITAMs). In some embodiments, compositions described herein comprise a TREM-2 modulator that in turn may comprise modulatory effects on DAP12 and/or SYK.

In some embodiments, compositions described herein can comprise a TREM-2 modulator, wherein the modulator is an inhibitor and/or depletor of TREM-2 expressing cells. In certain embodiments, an inhibitor and/or depletor of TREM-2 expressing cells can be or comprise anti-TREM-2 (PY314, Pionyr Immunotherapeutics).

In some embodiments, compositions described herein can comprise a TREM-2 modulator, wherein the TREM-2 modulator can be selected from but is not limited to: antibodies directed to the TREM-2 and fragments thereof which also modulate TREM-2, small molecules modulating the function, activity or expression of TREM-2, siRNAs directed to TREM-2 and/or TREM-2 negative regulators, shRNAs directed to TREM-2 and/or TREM-2 negative regulators, antisense oligonucleotides directed to TREM-2 and/or TREM-2 negative regulators, ribozymes directed to TREM-2 and/or TREM-2 negative regulators, aptamers which bind to and modulate TREM-2, fusion proteins between human IgG1 constant region and the extracellular domain of mouse TREM-2 or that of human TREM-2, and any combination thereof.

In some embodiments, a modulator of MDSC/neutrophil effector function may be or comprise an inhibitor of a TAM family receptor tyrosine kinase related signaling pathway. In some embodiments, such inhibitors may be directed to one or more TAM family receptor tyrosine kinases. In some embodiments, such inhibitors may be directed to TYRO3, AXL, MER (MERTK), and/or combinations thereof. In some embodiments, such inhibitors may be directed to one or more TAM family receptor tyrosine kinase ligands. In some embodiments, such inhibitors may be directed to GAS6 and/or Protein S. While not being bound by a particular theory, it is thought that TAM family receptor tyrosine kinases promote MDSC suppressive enzymatic capabilities, T-cell suppression activity, and migration to tumor-draining lymph nodes; see e.g., Holtzhausen et al., “TAM family receptor kinase inhibition reverses MDSC-mediated suppression and augments anti-PD-1 therapy in melanoma” Cancer Immunology Research (2019): 7(10):1672-1686; the contents of which are incorporated herein in their entirety by reference for the purposes described herein. On the other hand, while not being limited by a particular theory, it is thought that AXL and MER antagonize neutrophil counts and recruitment, and promote clearance of apoptotic and senescent neutrophils; see e.g., Fujimori et al., “The Axl receptor tyrosine kinase is a discriminator of macrophage function in the inflamed lung” Mucosal Immunology (2015): 8(5):1021-1030; Li et al., “The role of endothelial MERTK during the inflammatory response in lungs” PLOS One (2019): 14(12):e0225051; Bosurgi et al., “Paradoxical role of the proto-oncogene Axl and Mer receptor tyrosine kinases in colon cancer” PNAS (2013): 110(32):13091-6; and Hong et al., “Coordinate regulation of neutrophil homeostasis by liver X receptors in mice” The Journal of ClinicalInvestigation (2012): 122(1):337-347; the contents of each of which are incorporated herein in their entirety by reference for the purposes described herein. Additionally, while not being bound by a particular theory, it is thought that MER promotes clearance of apoptotic cancer cells within a tumor thus resulting in suppression of tumor immunogenicity and suppression of anti-tumor immunity. In some embodiments, an inhibitor of a TAM family receptor tyrosine kinase signaling pathway may be or comprise amuvatinib (MP-470, HK-56), bemcentinib (R428, BGB-324), bosutinib (SKI-606), cabozantinib (BMS-907351), dubermatinib (TP-0903), foretinib (EXEL-2880, GSK-1363089), gilteritinib (APS-2215), glesatinib (MGCD265), merestinib (LY-2801653), ningetinib (CT053PTSA), sitravatinib (MGCD516), 2-D08, BMS-777607, BPI-9016M, CEP-40783, CJ-2360, DS-1205B, LDC1267, MRX-2843, NPS-1034, ONO-7475, RU-301, RXDX-106, S49076, SGI-7079, TUN-00562, UNC569, UNC2025, UNC2250, UNC2541, UNC2881, UNC3133, UNC4203, UNC5293, anti-AXL antibodies (e.g., YW327.6S2), AXL decoy receptors (e.g., GL2I.T), or any combination thereof.

In some embodiments, a modulator of MDSC/neutrophil effector function may be or comprise an inhibitor of a leukocyte-associated immunoglobulin-like receptor (LAIR)-1 related signaling pathway. In some embodiments, such inhibitors may be directed to LAIR-1. In some embodiments, such inhibitors may be directed to a LAIR-1 ligand. In some embodiments, such inhibitors may be directed to collagen and/or C1q. While not being bound by a particular theory, it is thought that LAIR-1 suppresses neutrophil recruitment, formation of neutrophil extracellular traps (NETs), and neutrophil-driven inflammation; see e.g., Kumawat et al., “LAIR-1 limits neutrophilic airway inflammation” Frontiers in Immunology (2019): 10:842; Besteman et al., “Signal inhibitory receptor on leukocytes (SIRL)-1 and leukocyte-associated immunoglobulin-like receptor (LAIR)-1 regulate neutrophil function in infants” Clinical Immunology (2020): 211:108324; and Guo et al., “Role and mechanism of LAIR-1 in the development of autoimmune diseases, tumors, and malaria: a review” Current Research in Translational Medicine (2020): 68(3):119-124; the contents of each of which are incorporated herein in their entirety by reference for the purposes described herein. Additionally, while not being bound by a particular theory, it is thought that LAIR-1 promotes myeloid immunosuppression. In some embodiments, an inhibitor of LAIR-1 may be or comprise anti-LAIR-1 antibodies (e.g., NC410).

In some embodiments, a modulator of MDSC/neutrophil effector function may be or comprise a modulator of a leukocyte immunoglobulin-like receptor (LILR) (aka an immunoglobulin-like transcripts (ILT)) associated signaling pathway. In some embodiments, such modulators may be directed to LILRA1, LILRA2, LILRA3, LILRA4, LILRA5, LILRA6, LILRB1 (aka ILT2), LILRB2 (aka ILT4), LILRB3, LILRB4 (aka ILT3), and/or LILRB5. In some embodiments, such modulators may be directed to activating receptors LILRA2, LILRA3, LILRA5, or combinations thereof. In some embodiments, such modulators may be or comprise inhibitors of activating receptors LILRA2, LILRA3, LILRA5, or combinations thereof. In some embodiments, such modulators may be directed to inhibitory receptors LILRB1, LILRB2, LILRB3, or combinations thereof. In some embodiments, such modulators may be or comprise agonists of inhibitory receptors LILRB1, LILRB2, LILRB3, or combinations thereof. In some embodiments, such modulators may be directed to human leukocyte antigen G (HLA-G). While not being bound by a particular theory, it is thought that LILRs can stimulate or inhibit neutrophil function; see e.g., Marffy and McCarth “Leukocyte immunoglobulin-like receptors (LILRs) on human neutrophils: modulators of infection and immunity” Frontiers in Immunology (2020) 11:857; the contents of which are incorporated herein in their entirety by reference for the purposes described herein. Additionally, while not being bound by a particular theory, it is thought that in some instances ILT4 (and/or ILT2) can function by interaction with HLA-G; that in some instances ILT4 and HLA-G can suppress neutrophil phagocytosis and respiratory bursts, and/or that in some instances interaction between ILT4 and HLA-G can inhibit neutrophil function and/or induce immunosuppressive cells, such as myeloid suppressive cells; see e.g., Shiroishi et al. “Human inhibitory receptors Ig-like transcript 2 (ILT2) and ILT4 compete with CD8 for MHC class I binding and bind preferentially to HLA-G” Proc Natl Acad Sci USA (2003): 100(15):8856-8861; Baudhuin et al. “Exocytosis acts as a modulator of the ILT4-mediated inhibition of neutrophil functions” Proc Natl Acad Sci USA (2013): 110(44):17957-17962.; Rouas-Freiss et al. “The dual role of HLA-G in cancer” Journal of Immunology Research (2014): 2014:359748; and Rouas-Freiss et al. “Intratumor heterogeneity of immune checkpoints in primary renal cell cancer: focus on HLA-G/ILT2/ILT4” Onco Immunology (2017): 6(9):e1342023; the contents of each of which are incorporated herein in their entirety by reference for the purposes described herein. In some embodiments, a modulator (e.g., an inhibitor) of a LILR associated signaling pathway may be or comprise an anti-ILT2 antibody, anti-ILT3 antibody, anti-ILT4 antibody, anti-HLA-G antibody, or any combination thereof.

In some embodiments, a modulator of MDSC/neutrophil effector function may be or comprise an inhibitor of a c-Kit related signaling pathway. In some embodiments, such inhibitors may be directed to c-Kit. In some embodiments, such inhibitors may be directed to a c-Kit ligand. In some embodiments, such inhibitors may be directed to stem cell factor (SCF). While not being bound by a particular theory, it is thought that c-Kit promotes a tumor-elicited oxidative neutrophil phenotype, which promotes tumor growth; see e.g., Rice et al., “Tumour-elicited neutrophils engage mitochondrial metabolism to circumvent nutrient limitations and maintain immune suppression” Nature Communications (2018): 9(1):5099; and Mackey et al., “Neutrophil maturity in cancer” Frontiers in Immunology (2019): 10:1912; the contents of each of which are incorporated herein in their entirety by reference for the purposes described herein. In some embodiments, an inhibitor of a c-Kit related signaling pathway may be or comprise anti-c-Kit antibodies, anti-SCF antibodies, agerafenib (RXDX-105), amuvatinib (HPK-56, MP-470), apatinib (YN968D1), avapritinib (BLU-285), axitinib (AG-13736), cabozantinib (BMS-907351, XL-184), cediranib (AZD-2171), dasatinib (BMS-354825), dovitinib (TKI-258), erdafitinib (JNJ-42756493), imatinib (CGP-57148B), lenvatinib (E-7080), masitinib (AB-1010), motesanib (AMG-706), pazopanib (GW-786034), pexidartinib (CML-261, PLX-3397), ripretinib (DCC-2618), regorafenib (BAY-73-4506), sitravatinib (MGCD516), sorafenib (BAY-43-9006), sunitinib (SU-11248), tandutinib (CT 53518, MHLN518), telatinib (BAY-57-9352), tivozanib (AV-951, KIL-8951, KRN-951), AST-487, AZD2932, AZD3229, CS-2660 (JNJ-38158471), ISCK03, Ki20227, OSI-930, SU5614, UNC2025, or any combination thereof.

In some embodiments, a modulator of MDSC/neutrophil effector function may be or comprise an inhibitor of a MET related signaling pathway. In some embodiments, such inhibitors may be directed to MET. In some embodiments, such inhibitors may be directed to a MET ligand. In some embodiments, such inhibitors may be directed to hepatocyte growth factor (HGF). While not being bound by a particular theory, it is thought that MET promotes neutrophil recruitment and immunosuppression of T cells; see e.g., Glodde et al., “Reactive neutrophil responses dependent on the receptor tyrosine kinase c-MET limit cancer immunotherapy” Immunity (2017): 47(4):789-802.e9; the contents of which are incorporated herein in their entirety by reference for the purposes described herein. On the other hand, while not being bound by a particular theory, it is thought that MET promotes neutrophil recruitment and release of nitric oxide to promote killing of cancer cells; see e.g., Finisguerra et al., “MET is required for the recruitment of anti-tumoural neutrophils” Nature (2015): 522(7556):349-353; the contents of which are incorporated herein in their entirety by reference for the purposes described herein. In some embodiments, an inhibitor of a MET related signaling pathway may be or comprise anti-MET antibodies, anti-HGF antibodies, altiratinib (DCC-2701), amuvatinib (HPK-56, MP-470), bozitinib (PLB-1001, CBT-101), cabozantinib (BMS-907351), capmatinib (INCB-28060), crizotinib (PF-02341066), ensartinib (X-396), foretinib (GSK-1363089), glesatinib (MGCD-265), glumetinib (SC-C244), golvatinib (E-7050), merestinib (LY-2801653), ningetinib (CT053PTSA), norleual, pamufetinib (TAS-115), savolitinib (AZD6094, HMPL-504), sitravatinib, tepotinib (EMD-1214063), tivantinib (ARQ-197), AMG-1, AMG-208, AMG-337, AMG-458, ARRY-300, BAY-474, BMS-777607, BMS-794833, BPI-9016M, CBT-101, CT-711, DS-1205b, EMD-1204831, GNE-203, JNJ-38877605, JNJ-38877618 (OMO-1), MK-2461, MK-8033, NPS-1034, NVP-BVU972, PF-04217903, PHA-665752, RXDX-106 (CEP-40783), S49076, SAR125844, SCR-1481B1, SGX-523, SJF-8240, SOMCL-863, SOMG-833, SU11271, SU11274, SU11606, SYN1143, TPX-0022, and/or UNC2025, X-376, XL092, XL184, or any combination thereof.

In some embodiments, a modulator of MDSC/neutrophil effector function may be or comprise an inhibitor of interleukin-4 (IL-4) receptor (IL-4R) signaling. In some embodiments, such inhibitors may be directed to IL-4R. In some embodiments, such inhibitors may be directed to an IL-4R ligand. In some embodiments, such inhibitors may be directed to IL-4. In some embodiments, such inhibitors may be directed to JAK, Tyk2, and/or STAT6; see e.g., Bankaitis and Fingleton “Targeting IL4/IL4R for the treatment of epithelial cancer metastasis” (2015): 32(8):847-856; which is incorporated herein by reference in its entirety for the purposes described herein. While not being bound by a particular theory, it is thought that IL-4R signaling inhibits neutrophil migration and effector function, including production of neutrophil extracellular traps (NETs); see e.g., Heeb et al. “Evolution and function of interleukin-4 receptor signaling in adaptive immunity and neutrophils” Genes & Immunity (2020): 21:143-149; and Impellizzieri et al. “IL-4 receptor engagement in human neutrophils impairs their migration and extracellular trap formation” Translational and Clinical Immunology (2019): 144(1):267-279.E4; the contents of each of which are incorporated herein in their entirety by reference for the purposes described herein. In some embodiments, an inhibitor of IL-4R signaling may be or comprise anti-IL-4 antibodies, anti-IL-4R antibodies, JAK inhibitors, Tyk2 inhibitors, and/or STAT6 inhibitors (e.g., leflunomide and vorinostat), or any combination thereof.

In some embodiments, a modulator of MDSC/neutrophil effector function may be or comprise an inhibitor of monoamine oxidase A (MAO-A). While not being bound by a particular theory, it is thought that MAO-A promotes recruitment of neutrophils by promoting expression of chemokines (e.g., CXCL8 and CCL2), and promotes neutrophil-driven inflammation by suppression of anti-inflammatory cytokines (e.g., IL-10); see e.g., Ostadkarampour and Putnins “Monoamine oxidase inhibitors: a review of their anti-inflammatory therapeutic potential and mechanisms of action” Frontiers in Pharmacology (2021) 12:676239; the contents of which are incorporated herein in their entirety by reference for the purposes described herein. On the other hand, while not being bound by a particular theory, it is thought that MAO-A promotes tumor growth via tumor-associate macrophages (TAMs) and suppression of anti-tumor T cell immunity; see e.g., Wang et al. “Targeting monoamine oxidase A-regulated tumor-associated macrophage polarization for cancer immunotherapy” Nature Communications (2021) 12:3530; and Wang et al. “Targeting monoamine oxidase A for T cell-based cancer immunotherapy” Science Immunology (2021) 6(59):eabh2383; the contents of each of which are incorporated herein in their entirety by reference for the purposes described herein. In some embodiments, an inhibitor of MAO-A may be or comprise amiflamine (FLA-336), befloxatone (MD-370503), bifemelane (MCI-2016), brofaromine (CGP-11305A), clorgyline, coptisine, eprobemide, esuprone (LU-43839), harmine, isocarboxazid (Ro 5-0831), minaprine, mocolobemide, norharmane, pargyline (NSC 43798), phenelzine, pirlindole, tetrindole, toloxatone (MD69276), BW-1370U87, CX-157, Ro 41-1049, RS-8359, or any combination thereof.

In some embodiments, a modulator of MDSC/neutrophil effector function may be or comprise an inhibitor of complement component C5a and/or C5a receptor (C5aR). While not being bound by a particular theory, it is thought that C5a and C5aR promote neutrophil recruitment and activity via modulating neutrophil actin-cytoskeleton polymerization and reorganization; see e.g., Denk et al. “Complement C5a-induced changes in neutrophil morphology during inflammation” Scandinavian Journal of Immunology (2017) 86(3):143-155; and Schreiber et al. “C5a receptor mediates neutrophil activation and ANCA-induced glomerulonephritis” Journal of the American Society of Nephrology (2009) 20(2):289-298; the contents of each of which are incorporated herein in their entirety by reference for the purposes described herein. On the other hand, while not being bound by a particular theory, it is thought that C5a suppresses neutrophil effector function by suppression of TNFα production; see e.g., Riedemann et al. “Regulation by C5a of neutrophil activation during sepsis” Immunity (2003) 19(2):193-202; the contents of which are incorporated herein in their entirety by reference for the purposes described herein. In some embodiments, an inhibitor of C5a and/or C5aR may be or comprise an anti-C5a antibody and/or an anti-C5aR antibody.

In some embodiments, a modulator of MDSC/neutrophil effector function may be or comprise a corticosteroid. In some embodiments, a corticosteroid is a glucocorticoid (e.g., dexamethasone). In some embodiments, a corticosteroid is a corticosteroid prodrug or a corticosteroid metabolite. While not being bound by a particular theory, it is thought that glucocorticoids prevent inappropriate neutrophil accumulation by regulating by down-regulating CD62L expression on the neutrophil cell surface; and reduce neutrophil activation by suppression of NADPH-dependent ROS production, and reduction of COX and iNOS activities; see e.g., Ronchetti et al. “How glucocorticoids affect the neutrophil life” International Journal of Molecular Sciences (2018) 19(12):4090; the contents of which are incorporated herein in their entirety by reference for the purposes described herein. On the other hand, while not being bound by a particular theory, it is thought that glucocorticoids promote neutrophil maturation and mobilization leading to neutrophilia; promote neutrophil survival by several mechanisms (e.g., downregulation of the pro-apoptotic surface Fas receptor, upregulation of the pro-survival IAP protein family, upregulation of the anti-apoptotic Mcl-1 protein, and increased levels of the GR-P isoform); and promote inflammation via upregulated expression of IL-10 receptor and upregulated expression of leukotriene receptors (e.g., BLT1); see e.g., Ronchetti et al. (2018); and Saffar et al. “The molecular mechanisms of glucocorticoids-mediated neutrophil survival” Current Drug Targets (2011) 12(4):556-562; the contents of which are incorporated herein in their entirety by reference for the purposes described herein. Additionally, while not being bound by a particular theory, it is thought that acute local administration of a corticosteroid can reduce neutrophil accumulation and suppress neutrophil activity, while chronic use of corticosteroids by systemic exposure can promote neutrophilia and promote neutrophil-related inflammation. In some embodiments, a corticosteroid may be or comprise amcinonide, alclometasone dipropionate, beclometasone, betamethasone, betamethasone propionate, betamethasone sodium phosphate, betamethasone valerate, budesonide, ciclesonide, clobetasol propionate, clobetasone butyrate, cortisone acetate, cortisone acetate, desonide, desoximetasone, dexamethasone, dexamethasone sodium phosphate, diflorasone diacetate, diflucortolone valerate, fludrocortisone acetate, fluprednidene acetate, flunisolide, fluocortolone, fluocortolone caproate, fluocinonide, fluocinolone acetonide, fluticasone propionate, fluticasone furoate, flurandrenolide, fluticasone acetonide, fluorometholone, halcinonide, halobetasol, halometasone, halcinonide, hydrocortisone, hydrocortisone acetate, hydrocortisone aceponate, hydrocortisone buteprate, hydrocortisone butyrate, hydrocortisone valerate, methylprednisolone, methylprednisolone aceponate, mometasone, mometasone furoate, prednicarbate, prednisone, prednisolone, tixocortol pivalate, triamcinolone, triamcinolone acetonide, triamcinolone alcohol, or any combination thereof.

In some embodiments, a modulator of MDSC/neutrophil effector function may be or comprise an activator of glutamate-gated chloride channels and/or a positive allosteric effector of purinergic receptor P2X4 (P2RX4), purinergic receptor P2X7 (P2RX7), and/or alpha7 nicotinic acetylcholine receptor (α7 nAChR) (e.g., ivermectin). While not being bound by a particular theory, it is thought that ivermectin can promote anti-tumor activity at least in part by suppression of MDSC/neutrophil effector function. On the other hand, while not being bound by a particular theory, it is thought that ivermectin promotes the release of elastase by neutrophils and is capable of killing cancer cells in vitro in the absence of neutrophils; see e.g., Njoo et al. “Neutrophil activation in ivermectin-treated onchocerciasis patients” Clinical & Experimental Immunology (1993) 94(2):330-333; and Draganov et al. “Modulation of P2X4/P2X7/Pannexin-1 sensitivity to extracellular ATP via ivermectin induces a non-apoptotic and inflammatory form of cancer cell death” Science Reports (2015) 10(5):16222; the contents of each of which are incorporated herein in their entirety by reference for the purposes described herein. In some embodiments, an activator of glutamate-gated chloride channels and/or a positive allosteric effector of P2RX4, P2RX7, and/or α7 nAChR is or comprises avermectin, doramectin, milbemycin, selamectin, ivermectin, A-867744, PNU 120596, NS 1738, or any combination thereof.

In some embodiments, a modulator of MDSC/neutrophil effector function may be or comprise a beta-adrenergic receptor antagonist (beta blocker). In some embodiments, such modulators may be directed to beta-1 and/or beta-2 adrenergic receptors. While not being bound by a particular theory, it is thought that beta-adrenergic receptor signaling can be immunosuppressive, and treatment with beta blockers can have anti-tumor activity; see e.g., Kokolus et al. “Beta blocker use correlates with better overall survival in metastatic melanoma patients an improves the efficacy of immunotherapies in mice” Onco Immunology (2018) 7(3):e1405205; the contents of which are incorporated herein in their entirety by reference for the purposes described herein. Additionally, while not being bound by a particular theory, it is thought that beta blockers inhibit neutrophil migration and recruitment, reduce neutrophil to lymphocyte ratio (NLR), suppress neutrophil release of reactive oxygen species (ROS), and/or suppress neutrophil inflammatory responses; see e.g., Garcia-Prieto et al. “Neutrophil stunning by meoprolol reduces infarct size” Nature Communications (2017) 8:14780; Hussain “Nebivolol attenuates neutrophil lymphocyte ratio: a marker of subclinical inflammation in hypertensive patients” International Journal of Hypertension (2017) 7643628; Djanani et al. “Inhibition of neutrophil migration and oxygen free radical release by metipranolol and timolol” Pharmacology (2003) 68(4):198-203; Maglie et al. “Propranolol off-target: a new therapeutic option in neutrophil-dependent dermatoses?” Journal of Investigative Dermatology (2020) 140(12):2326-2329; Wrobel et al. “Propranolol induces a favourable shift of anti-tumor immunity in a murine spontaneous model of melanoma” Oncotarget (2016) 7(47):77825-77837; the contents of each of which are incorporated herein in their entirety by reference for the purposes described herein. In some embodiments, a beta blocker is or comprises propranolol, propranolol hydrochloride, timolol, timolol maleate, ancarolol, alprenolol, alprenolol hydrochloride, arotinolol, befunolol, butyryltimolol, bometolol hydrochloride, carteolol hydrochloride, carazolol, carvedilol, carvedilol phosphate hemihydrate, diacetolol, esmolol hydrochloride, labetalone hydrochloride, levobunolol hydrochloride, levobetaxolol hydrochloride, meipranolol hydrochloride, metipranolol hydrochloride, nadolol, penbutolol, pebutolol sulfate, pindolol, propafenone, pronethalol hydrochloride, teoprolol, todralazine, todralazine hydrochloride, atenolol, betaxolol, bisoprolol, bucindolol, celiprolol, landiolol, metoprolol, nebivolol, talinolol, or any combination thereof.

In some embodiments, a modulator of MDSC/neutrophil effector function may be or comprise an inhibitor of the renin-angiotensin system (RAS). In some embodiments, such inhibitors may be directed angiotensin converting enzyme (ACE). In some embodiments, such inhibitors may be directed to angiotensin II receptor. While not being bound by a particular theory, it is thought that RAS signaling can promote infiltration of tumor-promoting immune cells, and that ACE promotes NOX2 activity and/or ROS generation associated with cell activation in neutrophils; see e.g., Peter and Jain “Targeting the renin-angiotensin system to improve cancer treatment: implications for immunotherapy” Science Translational Medicine (2017) 9(410):eaan5616; and Khan et al. “Angiotensin-converting enzyme enhances the oxidative response and bactericidal activity of neutrophils” Blood 130(3):328-339; the contents of each of which are incorporated herein in their entirety by reference for the purposes described herein. On the other hand, while not being bound by a particular theory, it is thought that ACE functions to reduce the number of cells with MDSC phenotype and increases anti-tumor response; see e.g., Peter and Jain Science Translational Medicine (2017); the contents of which are incorporated herein in their entirety by reference for the purposes described herein. While not being bound by a particular theory, it is thought that angiotensin II receptor inhibitor treatment can cause neutropenia, reduce neutrophil to lymphocyte ratio (NLR), and suppress generation of reactive oxygen species (ROS) by leukocytes; see e.g., DIOVAN (valsartan) (prescribing information), East Hanover, NJ: Novartis Pharmaceuticals Corp, January 2017; Karaman et al. “The comparative effects of valsartan and amlodipine on vWf levels and N/L ratio in patients with newly diagnosed hypertension” Clinical and Experimental Hypertension (2013) 35(7):516-522; and Dandona et al. “Angiotensin II receptor blocker valsartan suppresses reactive oxygen species generation in leukocytes, nuclear factor-KB, in mononuclear cells of normal subjects: evidence of an anti-inflammatory action” The Journal of Clinical Endocrinology & Metabolism (2003) 88(9):4496-4501; the contents of each of which are incorporated herein in their entirety by reference for the purposes described herein. Additionally, while not being bound by a particular theory, it is thought that treatment with ACE inhibitors and/or angiotensin II receptor inhibitors can promote polarization of neutrophils toward an antitumoral phenotype; see e.g., Shrestha et al. “Angiotensin converting enzyme inhibitors and angiotension II receptor antagonist attenuate tumor growth via polarization of neutrophils toward an antitumor phenotype” Onco Immunology (2016) 5(1):e1067744; the contents of which are incorporated herein in their entirety by reference for the purposes described herein. In some embodiments, an ACE inhibitor is or comprises alacepril, arfalasin (HOE 409), benazepril, benazeprilat (CGS-14831), captopril (SQ-14225), ceronapril, cilazapril (Ro 31-2848), delapril, deserpidine, enalapril, fasidotril, fosinopril, foroxymithine, imidapril, indolapril, libenzapril, lisinopril (MK-521), methylsilanol acetyltyrosine, moexipril, moveltipril, pentopril, perindopril (S-9490), pivalopril, pivopril, ramipril (HOE-498), rentiapril (SA-446), quinapril, perindopril, spirapril, temocapril, trandolapril (RU44570), utibapril, vicenin 2, vicenin 3, zofenopril, BRL-36378, BW-A-575C, CI-925, CL-242817, CV-5975, GF-109, MDL-100240, REV-5975, REV-6134, Ro 31-2201, Ro 31-8472, SQ-27786, SQ-28854, and/or WF-10129. In some embodiments, an angiotensin II receptor inhibitor is or comprises valsartan, abitesartan, allisartan, azilsartan (TAK-536), candesartan, elisartan (HN-12206), embusartan, eprosartan, fimasartan (BR-A-657), fonsartan, irbesartan (BMS-186295), losartan, milfasartan, olmesartan (RNH-6270), olodanrigan (EMA-401), pratosartan, ripisartan, saprisartan, sparsentan (RE-021), tasosartan, telmisartan, zolasartan, A 81988, BIBS-39, BIBS-222, BMS 183920, BMS-248360, CGP-48369, CGP-42112, Dmp 811, DuP-532, E-4177, EMD-66684, EEXP-3174, EXP3892, EXP6803, EXP9270, L-158338, L-159282, LCZ-696, LY285434, ME-3221, MK-996, PD-123319, SC 51316, TA-606, TD-0212, WL 19, YM-358, ZD-6888, ZD-7155, or combinations thereof.

i) Dissemination of Cancer Cells & Promotion of Angiogenesis

In some embodiments, a modulator of MDSC/neutrophil effector function is or comprises a modulator of pathways implicated in neutrophil induced dissemination of cancer cells (e.g., residual cancer cells at a tumor resection site). The dissemination of cancer cells from a primary tumor site is an essential step in cancer metastasis. While not being bound by a particular theory, the extracellular matrix modifying capabilities of neutrophils and/or MDSCs are thought to be important contributors to cancer cell proliferation and metastasis.

In some embodiments, a modulator of MDSC/neutrophil effector function can be or comprise an inhibitor of neutrophil extracellular traps (NETs). In some embodiments, a modulator of neutrophil and/or MDSC cytology can be an inhibitor of NETosis. In certain embodiments, an inhibitor of NETosis can be or comprise a DNase and/or DNase analog (e.g., DNase I, and/or DNase 1-like 3).

In some embodiments, a modulator of MDSC/neutrophil effector function can be or comprise a modulator of matrix metalloproteinases (MMPs). While not being limited by a particular theory, it is thought the activity of MMPs is correlated with cancer initiation and progression, where they act to facilitate tissue remodeling, tumor progression, and metastasis; see e.g., Fields “The Rebirth of Matrix Metalloproteinase Inhibitors: Moving Beyond the Dogma” Cells (2019) 8(9): 984; the contents of which are incorporated herein in their entirety by reference for the purposes described herein. In certain embodiments, a modulator of MMP function can be or comprise JNJ0966 [N-(2-((2-methoxyphenyl)amino)-4′-methyl-[4,5′-bithiazol]-2′-yl)acetamide], NSC405020 [3,4-dichloro-N-(1-methylbutyl)benzamide], N-(4-fluorophenyl)-4-(4-oxo-3,4,5,6,7,8-hexahydroquinazolin-2-ylthio)butanamide, doxycycline, minocycline, ®-ND-336, triple-helical peptide inhibitors (THPIs), Mouse mAb REGA-3G12, AB0041, AB0046, GS-5745/andecaliximab, DX-2400, mAb 9E8, peptide P3 (P3a, FPGVPLDTHDVFQYREK), IS4 (acetyl-VMDGYPMP-NH2), or any combination thereof.

In some embodiments, a modulator of MDSC/neutrophil effector function can be or comprise a modulator of neutrophil elastase proteins. While not being limited by a particular theory, it is thought that neutrophil elastase function is upregulated in numerous cancer types, and correlates with poor prognosis, where elastase acts in a tumor and metastasis promoting manner. In certain embodiments, a modulator of neutrophil cytological function can be or comprise Sivelestat, EPI-hNE4, Prolastin, KRP-109, DX-890, Pre-elafin, MNEI, BAY 85-8501, POL6014, al-antitrypsin, HCH6-1, leupeptin hemisulfate, PF-429242, tranexamic acid, AKBA, carvacrol demethylnobiletin, AZD9668, or any combination thereof.

In some embodiments, a modulator of MDSC/neutrophil effector function can be or comprise a modulator of protein arginine deiminases 4 (PAD4). While not being limited by a particular theory, it is thought that PAD4 function is upregulated in numerous cancer types, and correlates with poor prognosis, where PAD4 acts in a tumor and metastasis promoting manner by facilitating mouse and human NET formation. In certain embodiments, an inhibitor of PAD4 can be or comprise F-amidine. In certain embodiments, an inhibitor of PAD4 can be or comprise Cl-amidine. In certain embodiments, an inhibitor of PAD4 can be or comprise GSK199, GSK484, BMS-P5, or any combination thereof.

In some embodiments, a modulator of MDSC/neutrophil effector function can be or comprise a modulator of Cathepsin G (CatG). While not being bound by a particular theory, it is thought that CatG is a chymotrypsin-like protease that is released upon degranulation of neutrophils, facilitating cancer cell dissemination and metastasis. In certain embodiments, an inhibitor of CatG can be or comprise a small polypeptide (e.g., mucus proteinase inhibitor, eglin c, and/or aprotinin). In certain embodiments, an inhibitor of CatG can be or comprise a serine protease inhibitor (e.g., al-antichymotrypsin). In certain embodiments, an inhibitor of CatG can be or comprise a negatively charged macromolecule (e.g., a polyanion DNA molecule shorter than 0.5kb) and/or mixtures of short nucleic acid fragments (e.g., defibrotide).

In some embodiments, a modulator of MDSC/neutrophil effector function may or comprises a modulator of pathways implicated in neutrophil induced angiogenesis. Angiogenesis and the supplying of tumor associated tissues with blood and/or nutrients is an essential step in cancer survival and/or metastasis. While not being bound by a particular theory, the extracellular matrix modifying capabilities of neutrophils and/or MDSCs are thought to be important contributors to angiogenesis, immune cell migration/infiltration, CXCL1 expression, cancer cell proliferation, and metastasis. In some embodiments, a modulator of MDSC/neutrophil effector function may be or comprise a modulator of VEGF/VEGFR related signaling pathways. In some embodiments, an inhibitor of neutrophil and/or MDSC facilitated promotion of angiogenesis may be or comprise a VEGF and/or VEGFR inhibitor. In some embodiments, a VEGF and/or VEGFR inhibitor may be or comprise r84, RAFL-2, GU81, paclitaxel, bevacizumab, aflibercept, pazopanib, cabozantinib, sunitinib, axitinib, lenvatinib, sorafenib, regorafenib, ponatinib, vandetanib, ramucirumab, brivanib alaninate (BMS-582664), cediranib (Recentin; Astrazeneca), motesanib (AMG 706, Amgen), linifanib (ABT 869 Abbott), functional derivatives thereof, or any combination thereof.

In some embodiments, a modulator of MDSC/neutrophil effector function can be or comprise a modulator of hepatocyte growth factor (HGF) and/or c-MET signaling. In some embodiments, an inhibitor of HGF signaling can be or comprise AM7, SU11274, BMS-777607, PF-02341066, AMG-458, JNJ-38877605, PF-04217903, Triazolopyrazine, MK-2461, Tivantinib (ARQ197), XL184, GSK/1363089/XL880, E7050, INCB28060, or combinations thereof.

In some embodiments, a modulator of MDSC/neutrophil effector function can be or comprise a modulator of angiopoietin signaling. In some embodiments, such modulators may be directed to an angiopoietin. In some embodiments, such modulators may be directed to ANG1 (ANGPT1) and/or ANG2 (ANGPT2). In some embodiments, such modulators may be directed to an angiopoietin receptor. In some embodiments, such modulators may be directed to TIE2. While not being bound by a particular theory, it is thought that angiopoietin signaling promotes neutrophil chemotaxis and synthesis of neutrophil extracellular traps (NETs), which can contribute to proinflammatory and proangiogenic activities; see e.g., Lavoie et al., “Synthesis of human neutrophil extracellular traps contributes to angiopoietin-mediated in vitro proinflammatory and proangiogenic activities” The Journal of Immunology (2018): 200(11):3801-3813; the contents of which are incorporated herein in their entirety by reference for the purposes described herein. In some embodiments, a modulator of angiopoietin signaling may be or comprise an inhibitor of an angiopoietin. In some embodiments, a modulator of angiopoietin signaling may be or comprise anti-ANG2 antibodies (e.g., MEDI3617), altiratinib (DCC-2701), cabozantinib (BMS-907351, XL-184), pexmetinib (ARRY-614), ponatinib, rebastinib (DCC-2036, DP-1919), regorafenib (BAY 73-4506), ripretinib (DCC-2618), trebananib (AMG-386), 2-MT 63, BAW 2881, BAY-826, BI 836880, CE-245677, CEP-11981, EOC317 (ACTB-1003), GW768505A, ODM-203, SB-633825, or combinations thereof.

Exemplary Biomaterial Preparations

Compositions comprising at least one modulator of myeloid-derived suppressive cell function (e.g., a modulator of neutrophil function) as described herein include at least one biomaterial preparation. In some embodiments, a biomaterial preparation described herein can form a polymer network which can act as a scaffold or depot for at least one modulator of myeloid-derived suppressive cell function (e.g., a modulator of neutrophil function) within the composition.

In some embodiments, a biomaterial preparation included in a composition described herein comprises one or more polymers (e.g., ones described herein herein). In certain embodiments, a biomaterial preparation included a composition described herein may comprise one or more positively-charged polymers. In certain embodiments, a biomaterial preparation included in a composition described herein may comprise one or more negatively-charged polymers. In certain embodiments, a biomaterial preparation included in a composition described herein may comprise one or more neutral polymers. In certain embodiments, a biomaterial preparation comprises one or more polymer components selected from: hyaluronic acid, alginate, chitosan, chitin, chondroitin sulfate, dextran, gelatin, collagen, starch, cellulose, polysaccharide, fibrin, poly-L-Lysine, methylcellulose, ethylene-vinyl acetate (EVA), poly(lactic-co-glycolic) acid (PLGA), polylactic acid (PLA), polyglycolic acid (PGA), polyethylene glycol (PEG), PEG diacrylate (PEGDA), disulfide-containing PEGDA (PEGSSDA), PEG dimethacrylate (PEGDMA), polydioxanone (PDO), polyhydroxybutyrate (PHB), poly(2-hydroxyethyl methacrylate) (pHEMA), polycaprolactone (PCL), poly(beta-amino ester) (PBAE), poly(ester amide), poly(propylene glycol) (PPG), poly(aspartic acid), poly(glutamic acid), poly(propylene fumarate) (PPF), poly(sebacic anhydride) (PSA), poly(trimethylene carbonate) (PTMC), poly(desaminotyrosyltyrosine alkyl ester carbonate) (PDTE), poly[bis(trifluoroethoxy)phosphazene], polyoxymethylene, single-wall carbon nanotubes, polyphosphazene, polyanhydride, poly(N-vinyl-2-pyrrolidone) (PVP), poly(vinyl alcohol) (PVA), poly(acrylic acid) (PAA), poly(methacrylic acid) (PMA), polyacetal, poly(alpha ester), poly(ortho ester), polyphosphoester, polyurethane, polycarbonate, polyamide, polyhydroxyalkanoate, polyglycerol, polyglucuronic acid, and/or combinations and/or derivatives thereof.

In some embodiments, a biomaterial preparation described herein is temperature-responsive, which thus permit in situ gelation at a target site in the absence of crosslinking treatments (e.g., introduction of UV radiation and/or chemical crosslinkers) that may have toxic or otherwise damaging effects for the recipient and/or for a payload that is included in or with a biomaterial. By way of example only, in some embodiments, a temperature-responsive biomaterial preparation as described herein is characterized in that it transitions from a precursor state (e.g., a liquid state or an injectable state) to a polymer network state that has a viscosity and/or storage modulus materially above that of the precursor state (e.g., a more viscous state or a hydrogel) when such a biomaterial preparation is exposed to a temperature at or above critical gelation temperature (CGT) for the biomaterial preparation. In some embodiments, a CGT for a provided biomaterial preparation is at least 10° C. or higher, including e.g. at least 10° C., at least 11° C., at least 12° C., at least 13° C., at least 14° C., at least 15° C., at least 16° C., at least 17° C., at least 18° C., at least 19° C., at least 20° C., at least 21° C., at least 22° C., at least 23° C., at least 24° C., at least 25° C., at least 26° C., at least 27° C., at least 28° C., at least 29° C., at least 30° C., at least 31° C., at least 32° C., 33° C., at least 34° C., at least 35° C., at least 36° C., at least 37° C., at least 38° C., at least 39° C., at least 40° C., or higher. In some embodiments, a CGT for a provided biomaterial preparation is about 10° C. to about 15° C. In some embodiments, a CGT for a provided biomaterial preparation is about 12° C. to about 17° C. In some embodiments, a CGT for a provided biomaterial preparation is about 14° C. to about 19° C. In some embodiments, a CGT for a provided biomaterial preparation is about 16° C. to about 21° C. In some embodiments, a CGT for a provided biomaterial preparation is about 18° C. to about 23° C. In some embodiments, a CGT for a provided biomaterial preparation is about 20° C. to about 25° C. In some embodiments, a CGT for a provided biomaterial preparation is about 22° C. to about 27° C. In some embodiments, a CGT for a provided biomaterial preparation is about 24° C. to about 29° C. In some embodiments, a CGT for a provided biomaterial preparation is about 26° C. to about 31° C. In some embodiments, a CGT for a provided biomaterial preparation is about 28° C. to about 33° C. In some embodiments, a CGT for a provided biomaterial preparation is about 30° C. to about 35° C. In some embodiments, a CGT for a provided biomaterial preparation is about 32° C. to about 37° C. In some embodiments, a CGT for a provided biomaterial preparation is about 34° C. to about 39° C. In some embodiments, a CGT for a provided biomaterial preparation is about 35° C. to about 39° C. In some embodiments, a CGT for a provided biomaterial preparation is at or near physiological temperature of a subject (e.g., a human subject) receiving such a biomaterial preparation.

In some embodiments, a provided biomaterial preparation is temperature-reversible. For example, in some embodiments, a provided biomaterial preparation is characterized in that it transitions from a precursor state (e.g., a liquid state or an injectable state) to a polymer network state that has a viscosity and/or storage modulus materially above that of the precursor state (e.g., a more viscous state or a hydrogel) when such a biomaterial preparation is exposed to a temperature at or above critical gelation temperature (CGT) for the biomaterial preparation; and it may revert from the polymer network state to a state that has a viscosity and/or storage modulus materially lower than that of the polymer network state (e.g., a liquid state or original state of a provided biomaterial preparation).

In some embodiments, a biomaterial preparation described herein does not comprise a chemical crosslinker. Those of skill in the art will appreciate that, in some embodiments, a chemical crosslinker is characterized in that it facilitates formation of covalent crosslinks between polymer chains. In some embodiments, a chemical crosslinker is or comprises a small-molecule crosslinker, which can be derived from a natural source or synthesized. Non-limiting examples of small-molecule crosslinkers include genipin, dialdehyde, glutaraldehyde, glyoxal, diisocyanate, glutaric acid, succinic acid, adipic acid, acrylic acid, diacrylate, etc.). In some embodiments, a chemical crosslinker may involve crosslinking using thiols (e.g., EXTRACEL©, HYSTEM©), methacrylates, hexadecylamides (e.g., HYMOVIS©), and/or tyramines (e.g., CORGEL©). In some embodiments, a chemical crosslinker may involve crosslinking using formaldehyde (e.g., HYLAN-A©), divinylsulfone (DVS) (e.g., HYLAN-B©), 1,4-butanediol diglycidyl ether (BDDE) (e.g., RESTYLANE©), glutaraldehyde, and/or genipin (see, e.g., Khunmanee et al. “Crosslinking method of hyaluronic-based hydrogel for biomedical applications” J Tissue Eng. 8: 1-16 (2017); the contents of which are incorporated herein in their entirety by reference for the purposes described herein). Accordingly, in some embodiments, crosslinks that form during the transition from a precursor state to a polymer network state do comprise covalent crosslinks.

In some embodiments, a temperature-responsive biomaterial preparation described herein is or comprises a poloxamer or a variant thereof. In some embodiments, a poloxamer or a variant thereof is present in a provided biomaterial preparation at a concentration of no more than 12.5% (w/w) (including, e.g., no more than 12% (w/w), no more than 11.5% (w/w), no more than 11% (w/w), no more than 10.5% (w/w), no more than 10% (w/w), no more than 9.5% (w/w), no more than 9% (w/w), no more than 8% (w/w)), no more than 7% (w/w), no more than 6% (w/w), no more than 5% (w/w), or no more than 4% (w/w). In some embodiments, a poloxamer or a variant thereof is present in a provided biomaterial preparation at a concentration of 5% (w/w) to 12.5% (w/w), or 8% (w/w) to 12.5% (w/w), or 5% (w/w) to 110% (w/w), or 5% (w/w) to 10% (w/w), or 6% (w/w) to 10% (w/w), or 8% (w/w) to 10% (w/w). In some embodiments, a poloxamer or a variant thereof is present in a provided biomaterial preparation at a concentration of 4% (w/w) to 12.5% (w/w), or 4% (w/w) to 11% (w/w), or 4% (w/w) to 10.5% (w/w), or 4% (w/w) to 10% (w/w). In some embodiments, a poloxamer or a variant thereof is present in a provided biomaterial preparation at a concentration of 5% (w/w) to 12.5% (w/w), or 5% (w/w) to 11% (w/w), or 5% (w/w) to 10.5% (w/w), or 5% (w/w) to 10% (w/w). In some embodiments, a poloxamer or a variant thereof is present in a provided biomaterial preparation at a concentration of 6% (w/w) to 12.5% (w/w), or 6% (w/w) to 11% (w/w), or 6% (w/w) to 10.5% (w/w), or 6% (w/w) to 10% (w/w).

(i) Exemplary Poloxamers

Poloxamer is typically a block copolymer comprising a hydrophobic chain of polyoxypropylene (e.g., polypropylene glycol, PPG, and/or poly(propylene oxide), PPO) flanked by two hydrophilic chains of polyoxyethylene (e.g., polyethylene glycol, PEG, and/or poly(ethylene oxide), PEO). Poloxamers are known by the trade names Synperonic, Pluronic, and/or Kolliphor. Generally, poloxamers are non-ionic surfactants, which in some embodiments may have a good solubilizing capacity, low toxicity, and/or high compatibility with cells, body fluids, and a wide range of chemicals.

In some embodiments, a poloxamer for use in accordance with the present disclosure may be a poloxamer known in the art. For example, as will be understood by a skilled person in the art, poloxamers are commonly named with the letter P (for poloxamer) followed by three digits: the first two digits multiplied by 100 give the approximate molecular mass of the polyoxypropylene chain, and the last digit multiplied by 10 gives the percentage polyoxyethylene content. By way of example only, P407 refers to a poloxamer with a polyoxypropylene molecular mass of 4000 g/mol and a 70% polyoxyethylene content). A skilled person in the art will also understand that for the Pluronic and Synperonic tradenames, coding of such poloxamers starts with a letter to define its physical form at room temperature (e.g., L=liquid, P=paste, F=flake (solid)) followed by two or three digits, wherein the first digit (two digits in a three-digit number) in the numerical designation, multiplied by 300, indicates the approximate molecular weight of the polyoxypropylene chain; and the last digit, multiplied by 10, gives the percentage polyoxyethylene content. By way of example only, L61 refers to a liquid preparation of poloxamer with a polyoxypropylene molecular mass of 1800 g/mol and a 10% polyoxyethylene content. In addition, as will be apparent to a skilled artisan, poloxamer 181 (P181) is equivalent to Pluronic L61 and Synperonic PE/L61.

In some embodiments, a poloxamer that may be included in a biomaterial preparation described herein may be or comprise Poloxamer 124 (e.g., Pluronic L44 NF), Poloxamer 188 (e.g., Pluronic F68NF), Poloxamer 181 (e.g., Pluronic L61), Poloxamer 182 (e.g., Pluronic L62), Poloxamer 184 (e.g., Pluronic L64), Poloxamer 237 (e.g., Pluronic F87 NF), Poloxamer 338 (e.g., Pluronic F108 NF), Poloxamer 331 (e.g., Pluronic L101), Poloxamer 407 (e.g., Pluronic F127 NF), or combinations thereof. In some embodiments, a provided biomaterial preparation can comprise at least two or more different poloxamers. Additional poloxamers as described in Table 1 of Russo and Villa “Poloxamer Hydrogels for Biomedical Applications” Pharmaceutics (2019) 11(12):671, the contents of which are incorporated herein by reference for the purposes described herein, may be also useful for biomaterial preparations described herein.

In some embodiments, a poloxamer that may be included in a biomaterial preparation described herein may be or comprise Poloxamer 407 (P407). In some embodiments, P407 is a triblock poloxamer copolymer having a hydrophobic PPO block flanked by two hydrophilic PEO blocks. The approximate length of the two PEO blocks is typically 101 repeat units, while the approximate length of the PPO block is 56 repeat units. In some embodiments, P407 has an average molecular weight of approximately 12,600 Da of which approximately 70% corresponds to PEO. In some embodiments, P407 can readily self-assemble to form micelles dependent upon concentration and ambient temperature. Without wishing to be bound by a particular theory, dehydration of hydrophobic PPO blocks combined with hydration of PEO blocks may lead to formation of spherical micelles, and subsequent packing of the micellar structure results in a 3D cubic lattice that constitutes the main structure of poloxamer hydrogels. They are also non-toxic, and stable, and are therefore suitable for use as controlled release of therapeutic agents. As appreciated by one of ordinary skill in the art, P407 concentrations in hydrogel formulations based on binary poloxamer/water mixtures are typically in the range from 16-20w/v %, with a value of approximately 18% w/v most frequently used. See, e.g., Pereia et al. “Formulation and Characterization of Poloxamer 407®: Thermoreversible Gel Containing Polymeric Microparticles and Hyaluronic Acid” Quim. Nova, Vol. 36, No. 8, 1121-1125 (2013), the contents of which are incorporated herein by reference in their entirety for purposes described herein.

In some embodiments, a poloxamer that may be included in a biomaterial preparation described herein may be or comprise a poloxamer as described in the International Patent Application No. PCT/US21/42110 filed Jul. 17, 2021, the entire content of which is incorporated herein by reference for purposes described herein.

In some embodiments, a provided temperature-responsive biomaterial preparation comprises a first polymer component (e.g., a poloxamer as described herein) and a second polymer component that is not a poloxamer. In some embodiments, a second polymer component may be present in a provided biomaterial preparation at a concentration of no more than 15% (w/w). In some embodiments, a second polymer component may be present in a provided biomaterial preparation at a concentration of no more than 10% (w/w), including, e.g., at a concentration of 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), or lower. In some embodiments, a second polymer component may be present in a provided biomaterial preparation at a concentration of at least 0.1% (w/w), including, e.g., at least 0.2% (w/w), at least 0.3% (w/w), at least 0.4% (w/w), at least 0.5% (w/w), at least 0.6% (w/w), at least 0.7% (w/w), at least 0.8% (w/w), at least 0.9% (w/w), at least 1% (w/w), at least 1.5% (w/w), at least 2% (w/w), at least 2.5% (w/w), at least 3% (w/w), at least 3.5% (w/w), at least 4% (w/w), at least 4.5% (w/w), at least 5% (w/w), at least 6% (w/w), at least 7% (w/w), at least 8% (w/w), at least 9% (w/w), at least 10% (w/w), or higher. In some embodiments, a second polymer component in a provided biomaterial preparation may be present at a concentration of 0.1% (w/w) to 10% (w/w), or 0.1% (w/w) to 8% (w/w), or 0.1% (w/w) to 5% (w/w), or 1% (w/w) to 5% (w/w). In some embodiments, a second polymer component may be present in a provided biomaterial preparation at a concentration of 0.5% (w/w) to 10% (w/w), or 0.5% (w/w) to 5% (w/w), or 1% (w/w) to 10% (w/w), or 1% (w/w) to 5% (w/w), or 2% to 10% (w/w).

In some embodiments, a second polymer component included in a provided biomaterial preparation may be or comprise at least one, including, e.g., at least two, at least three, at least four or more biocompatible and/or biodegradable polymer components. Examples of such a biocompatible and/or biodegradable polymer component include, but are not limited to immunomodulatory polymers, carbohydrate polymers (e.g., a polymer that is or comprises a carbohydrate, e.g., a carbohydrate backbone, including, e.g., but not limited to chitosan, alginate, hyaluronic acid, and/or variants thereof), polyacrylic acid, silica gels, polyethylenimine (PEI), polyphosphazene, and/or variants thereof), cellulose, chitin, chondroitin sulfate, collagen, dextran, gelatin, ethylene-vinyl acetate (EVA), fibrin, poly(lactic-co-glycolic) acid (PLGA), polylactic acid (PLA), polyglycolic acid (PGA), polyethylene glycol (PEG), PEG diacrylate (PEGDA), disulfide-containing PEGDA (PEGSSDA), PEG dimethacrylate (PEGDMA), polydioxanone (PDO), polyhydroxybutyrate (PHB), poly(2-hydroxyethyl methacrylate) (pHEMA), polycarboxybetaine (PCB), polysulfobetaine (PSB), polycaprolactone (PCL), poly(beta-amino ester) (PBAE), poly(ester amide), poly(propylene glycol) (PPG), poly(aspartic acid), poly(glutamic acid), poly(propylene fumarate) (PPF), poly(sebacic anhydride) (PSA), poly(trimethylene carbonate) (PTMC), poly(desaminotyrosyltyrosine alkyl ester carbonate) (PDTE), poly[bis(trifluoroethoxy)phosphazene], polyoxymethylene, single-wall carbon nanotubes, polyanhydride, poly(N-vinyl-2-pyrrolidone) (PVP), poly(vinyl alcohol) (PVA), poly(acrylic acid) (PAA), poly(methacrylic acid) (PMA), polyacetal, poly(alpha ester), poly(ortho ester), polyphosphoester, polyurethane, polycarbonate, polyamide, polyhydroxyalkanoate, polyglycerol, polyglucuronic acid, starch, variants thereof, and/or combinations thereof.

In some embodiments, a second polymer component included in a provided biomaterial preparation is or comprises an immunomodulatory polymer, e.g., a polymer that modulates one or more aspects of an immune response (e.g., a polymer that induces innate immunity agonism). In some embodiments, an immunomodulatory polymer may be or comprise a polymer agonist of innate immunity as described in International Patent Application No. PCT/US20/31169 filed May 1, 2020, (published as WO2020/223698A1), the entire content of which is incorporated herein by reference for purposes described herein.

In some embodiments, a second polymer component included in a provided biomaterial preparation may be or comprise a carbohydrate polymer, e.g., a polymer that is or comprises a carbohydrate, e.g., a carbohydrate backbone, including, e.g., but not limited to hyaluronic acid, chitosan, and/or variants thereof.

(ii) Exemplary Hyaluronic Acid and Variants Thereof

In some embodiments, a carbohydrate polymer included in a provided biomaterial preparation comprising a temperature-responsive polymer component (e.g., a poloxamer) is or comprises hyaluronic acid or a variant thereof. Hyaluronic acid (HA), also known as hyaluronan or hyaluronate, is a non-sulfated member of a class of polymers known as glycosaminoglycans (GAG) that is widely distributed in body tissues. HA is found as an extracellular matrix component of tissue that forms a pericellular coat on the surfaces of cells. In some embodiments, HA is a polysaccharide (which in some embodiments may be present as a salt, e.g., a sodium salt, a potassium salt, and/or a calcium salt) having a molecular formula of (C₁₄H₂₁NO₁₁)ₙ where n can vary according to the source, isolation procedure, and/or method of determination.

In some embodiments, HA that may be useful in accordance with the present disclosure can be isolated or derived from many natural sources. For example, in some embodiments, HA can be isolated or derived from, including, e.g., human umbilical cord, rooster combs, and/or connective matrices of vertebrate organisms. In some embodiments, HA can be isolated or derived from a capsular component of bacteria such as Streptococci. See, e.g., Kendall et al, (1937), Biochem. Biophys. Acta, 279, 401-405; the contents of which are incorporated herein in their entirety by reference for the purposes described herein. In some embodiments, HA and/or variants thereof can be produced via microbial fermentation. In some embodiments, HA and/or variants thereof may be a recombinant HA or variants thereof, for example, produced using Gram-positive and/or Gram-negative bacteria as a host, including, e.g., but not limited to Bacillus sp., Lactococcos lactis, Agrobacterium sp., and/or Escherichia coli.

In some embodiments, HA or variants thereof that may be included in a provided biomaterial preparation can have a low molecular weight, for example, an average molecular weight of 500 kDa or less, including, e.g., 450 kDa, 400 kDa, 350 kDa, 300 kDa, 250 kDa, 200 kDa, 150 kDa, 100 kDa, 50 kDa, or less. In some embodiments, HA or variants thereof that may be included in a provided biomaterial preparation may have an average molecular weight of about 100 kDa to about 150 kDa. In some embodiments, HA or variants thereof that may be included in a provided biomaterial preparation may have an average molecular weight of about 250 kDa to about 350 kDa. In some embodiments, HA or variants thereof that may be included in a provided biomaterial preparation may have an average molecular weight of about 300 kDa to about 400 kDa.

In some embodiments, HA or variants thereof that may be included in a provided biomaterial preparation can have a high molecular weight, for example, an average molecular weight of greater than 500 kDa or higher, including, e.g., 550 kDa, 600 kDa, 650 kDa, 700 kDa, 750 kDa, 800 kDa, 850 kDa, 900 kDa, 950 kDa, 1 MDa, 1.1 MDa, 1.2 MDa, 1.3 MDa, 1.4 MDa, 1.5 MDa, 1.6 MDa, 1.7 MDa, 1.8 MDa, 1.9 MDa, 2 MDa, 2.5 MDa, 3 MDa, 3.5 MDa, 4 MDa, 4.5 MDa, or higher. In some embodiments, HA or variants thereof that may be useful in accordance with the present disclosure may have an average molecular weight of about 600 kDa to about 900 kDa. In some embodiments, HA or variants thereof that may be useful in accordance with the present disclosure may have an average molecular weight of about 700 kDa to about 900 kDa. In some embodiments, HA or variants thereof that may be useful in accordance with the present disclosure may have an average molecular weight of about 500 kDa to about 800 kDa. In some embodiments, HA or variants thereof that may be useful in accordance with the present disclosure may have an average molecular weight of about 600 kDa to about 900 kDa. In some embodiments, HA or variants thereof that may be useful in accordance with the present disclosure may have an average molecular weight of about 700 kDa to about 800 kDa. In some embodiments, HA or variants thereof that may be useful in accordance with the present disclosure may have an average molecular weight of about 1 MDa to about 3 MDa.

In some embodiments, a provided biomaterial preparation comprises a hyaluronic acid variant. In some embodiments, a hyaluronic acid variant is water-soluble. In some embodiments, a hyaluronic acid variant may be a chemically modified hyaluronic acid, e.g., in some embodiments, hyaluronic acid is esterified. Examples of chemical modifications to hyaluronic acid include, but are not limited to, addition of thiol, haloacetate, butanediol, diglycidyl, ether, dihydrazide, aldehyde, glycan, and/or tyramine functional groups. Additional hyaluronic acid modifications and variants are known in the art. See e.g., Highley et al., “Recent advances in hyaluronic acid hydrogels for biomedical applications” Curr Opin Biotechnol (2016) August 40:35-40; Burdick & Prestwich, “Hyaluronic acid hydrogels for biomedical applications” Advanced Materials (2011); Prest which, “Hyaluronic acid-based clinical biomaterials derived for cell and molecule delivery in regenerative medicine” J. Control Release (2011) Oct. 30; 155(2): 193-199; each of which are incorporated herein by reference in their entirety for the purposes described herein.

In some embodiments, a provided biomaterial preparation comprises a hyaluronic acid or variant thereof as described in the International Patent Application No. PCT/US21/42110 filed Jul. 17, 2021, the entire content of which is incorporated herein by reference for purposes described herein.

In some embodiments, a provided biomaterial preparation comprises at least one poloxamer present at a concentration of 12.5% (w/w) or below (e.g., as described herein) and a second polymer component, which may be or comprise hyaluronic acid or variant thereof. In some such embodiments, HA or a variant thereof may be present in a provided polymer combination preparation at a concentration of about 10% (w/w) or lower, including, e.g., 9% (w/w), 8% (w/w), 7% (w/w), 6% (w/w), 5% (w/w), 4% (w/w), 3% (w/w), 2% (w/w), or 1% (w/w) or lower. In some embodiments, HA or a variant thereof may be present in a provided polymer combination preparation at a concentration of about 0.5% (w/w) to about 5% (w/w), e.g., at a concentration of 0.5% (w/w), 0.6% (w/w), 0.7% (w/w), 0.8% (w/w), 0.9% (w/w), 1% (w/w), 1.5% (w/w), 2% (w/w), 2.5% (w/w), 3% (w/w), 3.5% (w/w), 4% (w/w), 4.5% (w/w), or 5% (w/w). In some embodiments, HA or a variant thereof having a low molecular weight (e.g., as described herein) may be present in a provided biomaterial preparation at a concentration of at least about 1.5% (w/w) or higher, including, e.g., at least 2% (w/w), at least 2.5% (w/w), at least 3% (w/w), at least 4% (w/w), at least 5% (w/w), at least 6% (w/w), at least 7% (w/w), at least 8% (w/w), at least 9% (w/w), or higher. In some embodiments, HA or a variant thereof having a low molecular weight (e.g., as described herein) may be present in a provided biomaterial preparation at a concentration of about 1.5% (w/w) to about 5% (w/w). In some embodiments, HA or a variant thereof having a low molecular weight (e.g., as described herein) may be present in a provided biomaterial preparation at a concentration of about 0.5% (w/w) to about 10% (w/w). In some embodiments, HA or a variant thereof having a low molecular weight (e.g., as described herein) may be present in a provided biomaterial preparation at a concentration of about 1% (w/w) to about 10% (w/w) or about 1.5% (w/w) to about 10% (w/w). In some embodiments, HA or a variant thereof having a low molecular weight (e.g., as described herein) may be present in a provided biomaterial preparation at a concentration of about 0.7% (w/w) to about 4% (w/w) or about 1.5% (w/w) to about 4% (w/w). In some embodiments, HA or a variant thereof having a low molecular weight (e.g., as described herein) may be present in a provided biomaterial preparation at a concentration of about 3% (w/w) to about 7% (w/w). In some embodiments, HA or a variant thereof having a high molecular weight (e.g., as described herein) may be present in a provided biomaterial preparation at a concentration of 2% (w/w) or lower, including, e.g., 1.5% (w/w), 1.25% (w/w), 1% (w/w), or lower. In some embodiments, HA or a variant thereof having a high molecular weight (e.g., as described herein) may be present in a provided biomaterial preparation at a concentration of about 0.5% (w/w) to about 3% (w/w).

(iii) Exemplary Chitosan and Variants Thereof

In some embodiments, a carbohydrate polymer included in a provided biomaterial preparation comprising a temperature-responsive polymer (e.g., a poloxamer as described herein) may be or comprise chitosan or a variant thereof. Examples of chitosan and/or variants thereof that can be included in a biomaterial preparation described herein include, but are not limited to chitosan, chitosan salts (e.g., chitosan HCl, chitosan chloride, chitosan lactate, chitosan acetate, chitosan glutamate), alkyl chitosan, aromatic chitosan, carboxyalkyl chitosan (e.g., carboxymethyl chitosan), hydroxyalkyl chitosan (e.g., hydroxypropyl chitosan, hydroxyethyl chitosan), aminoalkyl chitosan, acylated chitosan, phosphorylated chitosan, thiolated chitosan, quaternary ammonium chitosan (e.g., N-(2-hydroxyl) propyl-3-trimethyl ammonium chitosan chloride), guanidinyl chitosan, chitosan oligosaccharide, glycated chitosan (e.g., N-dihydrogalactochitosan), chitosan poly(sulfonamides), chitosan-phenylsuccinic acid (e.g., products formed from the reaction of phenylsuccinic anhydride or a variant thereof (including, e.g., 2-phenylsuccinic anhydride, 2-phenylsuccinic acid derivatives, 2-O-acetyl L-Malic anhydride, etc.) and chitosan (e.g., Chitosan Phenylsuccinic acid hemi-amide—ring opened amide-carboxylic acid derivative), and variants or combinations thereof. In some embodiments, a carbohydrate polymer included in a provided biomaterial preparation comprising poloxamer (e.g., as described herein) may be or comprise carboalkyl chitosan (e.g., carboxymethyl chitosan).

Those skilled in the art will appreciate that, in some cases, chitosan and/or variants thereof can be produced by deacetylation of chitin. In some embodiments, chitosan or variants thereof included in a biomaterial preparation comprising poloxamer (e.g., as described herein) is characterized by degree of deacetylation (i.e., percent of acetyl groups removed) of at least 70% or above, including, e.g., at least 75%, at least 80%, at least 85%, at least 90%, at least 95%, or higher (including up to 100%). In some embodiments, a chitosan or variants thereof is characterized by degree of deacetylation of no more than 99%, no more than 95%, no more than 90%, no more than 85%, no more than 80%, no more than 75% or lower. Combinations of the above-mentioned ranges are also possible. For example, a chitosan or variants thereof may be characterized by degree of deacetylation of 80%-95%, 70%-95%, or 75%-90%. As will be recognized by one of those skilled in the art, degree of deacetylation (% DA) can be determined by various methods known in the art, e.g., in some cases, by NMR spectroscopy.

In some embodiments, chitosan or variants thereof included in a biomaterial preparation described herein may have an average molecular weight of at least 5 kDa or higher, including, e.g., at least 10 kDa or higher, including, e.g., at least 20 kDa, at least 30 kDa, at least 40 kDa, at least 50 kDa, at least 60 kDa, at least 70 kDa, at least 80 kDa, at least 90 kDa, at least 100 kDa, at least 110 kDa, at least 120 kDa, at least 130 kDa, at least 140 kDa, at least 150 kDa, at least 160 kDa, at least 170 kDa, at least 180 kDa, at least 190 kDa, at least 200 kDa, at least 210 kDa, at least 220 kDa, at least 230 kDa, at least 240 kDa, at least 250 kDa, at least 260 kDa, at least 270 kDa, at least 280 kDa, at least 290 kDa, at least 300 kDa, at least 350 kDa, at least 400 kDa, at least 500 kDa, at least 600 kDa, at least 700 kDa, or higher. In some embodiments, chitosan or variants thereof included in a biomaterial preparation described herein may have an average molecular weight of no more than 750 kDa or lower, including, e.g., no more than 700 kDa, no more than 600 kDa, no more than 500 kDa, no more than 400 kDa, no more than 300 kDa, no more than 200 kDa, no more than 100 kDa, no more than 50 kDa, or lower. Combinations of the above-mentioned ranges are also possible. For example, in some embodiments, chitosan or variants thereof included in a biomaterial preparation described herein is characterized by an average molecular weight of 10 kDa to 700 kDa, or 20 kDa to 700 kDa, or 30 kDa to 500 kDa, or 150 kDa to 600 kDa, or 150 kDa to 400 kDa, or 50 kDa to 150 kDa, or 10 kDa to 50 kDa. In some embodiments, chitosan or variants thereof included in a biomaterial preparation described herein is characterized by an average molecular weight of 20 kDa to 700 kDa, or 30 kDa to 500 kDa. As noted herein, an average molecular weight may be a number average molecular weight, weight average molecular weight, or peak average molecular weight.

In some embodiments, chitosan or variants thereof included in a biomaterial preparation described herein is characterized by a molecular weight distribution in a range of 10 kDa to 700 kDa, or 20 kDa or 700 kDa, or 30 kDa to 500 kDa, or 150 kDa to 600 kDa, or 150 kDa to 400 kDa, or 50 kDa to 150 kDa, or 10 kDa to 50 kDa. In some embodiments, chitosan or variants thereof included in a biomaterial preparation described herein is characterized by a molecular weight distribution in a range of 20 kDa to 700 kDa, or 30 kDa to 500 kDa.

In some embodiments, chitosan or variants thereof included in a biomaterial preparation described herein may be characterized by a viscosity of no more than 3500 mPa·s or lower, including, e.g., no more than 3000 mPa·s, no more than 2500 mPa·s, no more than 2000 mPa·s, no more than 1500 mPa·s, no more than 1000 mPa·s, no more than 500 mPa·s, no more than 250 mPa·s, no more than 200 mPa·s, no more than 150 mPa·s, no more than 100 mPa·s, no more than 75 mPa·s, no more than 50 mPa·s, no more than 25 mPa·s, no more than 20 mPa·s, no more than 15 mPa·s, no more than 10 mPa·s, or lower. In some embodiments, chitosan or variants thereof may be characterized by a viscosity of at least 5 mPa·s or higher, including, e.g., at least 10 mPa·s, at least 20 mPa·s, at least 30 mPa·s, at least 40 mPa·s, at least 50 mPa·s, at least 60 mPa·s, at least 70 mPa·s, at least 80 mPa·s, at least 90 mPa·s, at least 100 mPa·s, at least 125 mPa·s, at least 150 mPa·s, at least 175 mPa·s, at least 250 mPa·s, at least 500 mPa·s, at least 1000 mPa·s, at least 1500 mPa·s, at least 2000 mPa·s, at least 2500 mPa·s, or higher. Combinations of the above-mentioned ranges are also possible. For example, in some embodiments, such a viscous polymer solution of or comprising chitosan or variants thereof may be characterized by a viscosity of 5 mPa·s to 3000 mPa·s, or 5 mPa·s to 300 mPa·s, 5 mPa·s to 200 mPa·s, or 20 mPa·s to 200 mPa·s, or 5 mPa·s to 20 mPa·s. In some embodiments, viscosity of chitosan or variants thereof described herein is measured at 1% in 1% acetic acid at 20° C.

In some embodiments, a biomaterial preparation described herein comprises at least one or more (e.g., 1, 2, 3 or more) chitosan and/or variants thereof (including, e.g., modified chitosan and/or salts of chitosan or modified chitosan such as a chloride salt or a glutamate salt). For example, in some embodiments, chitosan and/or variants thereof (including, e.g., modified chitosan and/or salts of chitosan or modified chitosan such as a chloride salt or a glutamate salt) may be characterized by degree of deacetylation of 70%-95%, or 75%-90%, or 80%-95%, or greater than 90%. In some embodiments, chitosan and/or variants thereof (including, e.g., modified chitosan and/or salts of chitosan or modified chitosan such as a chloride salt or a glutamate salt) may be characterized by an average molecular weight of 10 kDa to 700 kDa, 20 kDa to 600 kDa, 30 kDa to 500 kDa, 150 kDa to 400 kDa, or 200 kDa to 600 kDa (e.g., measured as chitosan or chitosan salt, e.g., chitosan acetate). In some embodiments, chitosan and/or variants thereof (including, e.g., modified chitosan and/or salts of chitosan or modified chitosan such as a chloride salt or a glutamate salt) may be characterized by a molecular weight distribution in the range of 10 kDa to 700 kDa, 20 kDa to 600 kDa, 30 kDa to 500 kDa, 150 kDa to 400 kDa, or 200 kDa to 600 kDa (e.g., measured as chitosan or chitosan salt, e.g., chitosan acetate). In some embodiments, chitosan and/or variants thereof (including, e.g., salts thereof such as a chloride salt or a glutamate salt) may be characterized by a viscosity ranging from 5 to 3000 mPa·s, or 5 to 300 mPa·s, or 20 to 200 mPa·s. In some embodiments, such chitosan and/or variants thereof (including, e.g., salts thereof such as a chloride salt or a glutamate salt) may be or comprise PROTASAN™ UltraPure chitosan chloride and/or chitosan glutamate salt (e.g., obtained from NovoMatrix®, which is a business unit of FMC Health and Nutrition (now a part of Du Pont; Product No. CL 113, CL 114, CL 213, CL 214, G 113, G 213, G 214). In some embodiments, such chitosan and/or variants thereof (including, e.g., salts thereof such as a chloride salt or a glutamate salt) may be or comprise chitosan, chitosan oligomers, and/or variants thereof (including, e.g., Chitosan HCl, carboxymethyl chitosan, chitosan lactate, chitosan acetate), e.g., obtained from Heppe Medical Chitosan GMBH (e.g., Chitoceuticals® or Chitoscience®).

In some embodiments, chitosan or variants thereof included in a biomaterial preparation described herein is or comprises carboxyalkyl chitosan (e.g., carboxymethyl chitosan) that is characterized by at least one or all of the following characteristics: (1) degree of deacetylation of 80%-95%; (ii) an average molecular weight of 30 kDa to 500 kDa; or a molecular weight distribution of 30 kDa to 500 kDa; and (iii) a viscosity ranging from 5 to 300 mPa·s.

In some embodiments, chitosan or variants thereof included in a biomaterial preparation described herein is or comprises a variant of chitosan (e.g., as described herein). In some embodiments, such a variant of chitosan may include chemical modification(s) of one or more chemical moieties, e.g., hydroxyl and/or amino groups, of the chitosan chains. In some embodiments, such a variant of chitosan is or comprises a modified chitosan such as, e.g., but not limited to a glycated chitosan (e.g., chitosan modified by addition of one or more monosaccharide or oligosaccharide side chains to one or more of its free amino groups). Exemplary glycated chitosan that are useful herein include, e.g., but are not limited to ones described in U.S. Pat. Nos. 5,747,475, 6,756,363, WO 2013/109732, US 2018/0312611, and US 2019/0002594, the contents of each of which are incorporated herein by reference for the purposes described herein.

In some embodiments, chitosan or variants thereof included in a biomaterial preparation described herein is or comprises chitosan conjugated with a polymer that increases its solubility in aqueous environment (e.g., a hydrophilic polymer such as polyethylene glycol).

In some embodiments, chitosan or variants thereof included in a biomaterial preparation described herein is or comprises thiolated chitosan. Various modifications to chitosans, e.g., but not limited to carboxylation, PEGylation, galactosylation (or other glycations), and/or thiolation are known in the art, e.g., as described in Ahmadi et al. Res Pharm Sci., 10(1): 1-16 (2015), the contents of which are incorporated herein by reference for the purposes described herein. Those skilled in the art reading the present disclosure will appreciate that other modified chitosans can be useful for a particular application in which a method is being practiced.

In some embodiments, a provided biomaterial preparation comprises a chitosan or variant thereof as described in the International Patent Application No. PCT/US21/42110 filed Jul. 17, 2021, the entire content of which is incorporated herein by reference for purposes described herein.

In some embodiments, a provided biomaterial preparation comprises at least one poloxamer present at a concentration of 12.5% or below (e.g., as described herein) and a second polymer component, which may be or comprise chitosan or variant thereof. In some such embodiments, chitosan or a variant thereof may be present in a provided biomaterial preparation at a concentration of about 10% (w/w) or lower, including, e.g., 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) or lower. In some embodiments, chitosan or a variant thereof may be present in a provided biomaterial preparation at a concentration of 0.1% (w/w) to 10% (w/w), or 0.10% (w/w) to 8% (w/w), or 0.10% (w/w) to 5% (w/w), or 1% (w/w) to 5% (w/w), or about 10% (w/w) to about 30% (w/w).

In some embodiments, a biomaterial preparation described herein may be or comprise a polymer combination preparation as described in the International Patent Application No. PCT/US21/42110 filed Jul. 17, 2021, the entire content of which is incorporated herein by reference for purposes described herein. For example, in some embodiments, a biomaterial preparation described herein may comprise poloxamer (e.g., P407) and hyaluronic acid. In some embodiments, a biomaterial preparation described herein may comprise poloxamer (e.g., P407), hyaluronic acid, and chitosan or a variant thereof.

(iv) Exemplary Characteristics and/or Properties of Provided Biomaterial Compositions

In certain embodiments, a provided composition comprises a biomaterial that can extend the release of a modulator of myeloid-derived suppressive cell function (e.g., modulator of neutrophil function) when delivered to a target site (e.g., a tumor resection site) relative to administration of the same a modulator of myeloid-derived suppressive cell function (e.g., modulator of neutrophil function) in solution. In certain embodiments, a biomaterial (e.g., a polymeric biomaterial described herein) extends the release of a modulator of myeloid-derived suppressive cell function (e.g., modulator of neutrophil function) at a tumor resection site relative to administration of the same modulator of myeloid-derived suppressive cell function (e.g., modulator of neutrophil function) in solution by at least 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 11 hours, 12 hours, 18 hours, 24 hours, 2 days, 3 days, 4 days, 5 days, 6 days, 7 days, 2 weeks, 3 weeks, or 4 weeks. In some embodiments, a biomaterial (e.g., a polymeric biomaterial described herein) extends release of a modulator of myeloid-derived suppressive cell function (e.g., modulator of neutrophil function) so that, when assessed at a specified time point after administration, more modulator of myeloid-derived suppressive cell function (e.g., modulator of neutrophil function) is present in a tumor resection site relative to the levels observed when the modulator of myeloid-derived suppressive cell function (e.g., modulator of neutrophil function) is administered in solution. For example, in some embodiments, when assessed at 24 hours after administration, the amount of a modulator of myeloid-derived suppressive cell function (e.g., modulator of neutrophil function) released to and present in a tumor resection site is at least 30% more (including, e.g., at least 40%, at least 50%, at least 60%, at least 70%, at least 80%, at least 90%, or more) than that is observed when the modulator of myeloid-derived suppressive cell function (e.g., modulator of neutrophil function) is administered in solution. In some embodiments, when assessed at 48 hours after administration, the amount of a modulator of myeloid-derived suppressive cell function (e.g., modulator of neutrophil function) released to and present in a tumor resection site is at least 30% more (including, e.g., at least 40%, at least 50%, at least 60%, at least 70%, at least 80%, at least 90%, or more) than that is observed when the modulator of myeloid-derived suppressive cell function (e.g., modulator of neutrophil function) is administered in solution. In some embodiments, when assessed at 3 days after administration, the amount of a modulator of myeloid-derived suppressive cell function (e.g., modulator of neutrophil function) released to and present in a tumor resection site is at least 30% more (including, e.g., at least 40%, at least 50%, at least 60%, at least 70%, at least 80%, at least 90%, or more) than that is observed when the modulator of myeloid-derived suppressive cell function (e.g., modulator of neutrophil function) is administered in solution. In some embodiments, when assessed at 5 days after administration, the amount of a modulator of myeloid-derived suppressive cell function (e.g., modulator of neutrophil function) released to and present in a tumor resection site is at least 30% more (including, e.g., at least 40%, at least 50%, at least 60%, at least 70%, at least 80%, at least 90%, or more) than that is observed when the modulator of myeloid-derived suppressive cell function (e.g., modulator of neutrophil function) is administered in solution.

In some embodiments, compositions comprising a biomaterial preparation described herein (e.g., a polymeric biomaterial in a precursor state or in a polymer network state) can be characterized by a viscosity of no more than 25,000 mPa·s or lower, including, e.g., no more than 24,000 mPa·s, no more than 23,000 mPa·s, no more than 22,000 mPa·s, no more than 21,000 mPa·s, no more than 20,000 mPa·s, no more than 19,000 mPa·s, no more than 18,000 mPa·s, no more than 17,000 mPa·s, no more than 16,000 mPa·s, no more than 15,000 mPa·s, no more than 14,000 mPa·s, no more than 13,000 mPa·s, no more than 12,000 mPa·s, no more than 11,000 mPa·s, no more than 10,000 mPa·s, no more than 9000 mPa·s, no more than 8000 mPa·s, no more than 7000 mPa·s, no more than 6000 mPa·s, no more than 5000 mPa·s, no more than 4000 mPa·s, no more than 3500 mPa·s, no more than 3000 mPa·s, no more than 2500 mPa·s, no more than 2000 mPa·s, no more than 1500 mPa·s, no more than 1000 mPa·s, no more than 500 mPa·s, no more than 250 mPa·s, no more than 200 mPa·s, no more than 150 mPa·s, no more than 100 mPa·s, no more than 75 mPa·s, no more than 50 mPa·s, no more than 25 mPa·s, no more than 20 mPa·s, no more than 15 mPa·s, no more than 10 mPa·s, or lower. In some embodiments, compositions comprising a biomaterial preparation described herein (e.g., a polymeric biomaterial in a precursor state or in a polymer network state such as, e.g., a viscous solution) may be characterized by a viscosity of at least 5 mPa·s or higher, including, e.g., at least 10 mPa·s, at least 20 mPa·s, at least 30 mPa·s, at least 40 mPa·s, at least 50 mPa·s, at least 60 mPa·s, at least 70 mPa·s, at least 80 mPa·s, at least 90 mPa·s, at least 100 mPa·s, at least 125 mPa·s, at least 150 mPa·s, at least 175 mPa·s, at least 250 mPa·s, at least 500 mPa·s, at least 1000 mPa·s, at least 1500 mPa·s, at least 2000 mPa·s, at least 2500 mPa·s, at least 3000 mPa·s, at least 4000 mPa·s, at least 5000 mPa·s, at least 6000 mPa·s, at least 7000 mPa·s, at least 8000 mPa·s, at least 9000 mPa·s, at least 10,000 mPa·s, at least 11,000 mPa·s, at least 12,000 mPa·s, at least 13,000 mPa·s, at least 14,000 mPa·s, at least 15,000 mPa·s, at least 16,000 mPa·s, at least 17,000 mPa·s, at least 18,000 mPa·s, at least 19,000 mPa·s, at least 20,000 mPa·s, at least 21,000 mPa·s, at least 22,000 mPa·s, at least 23,000 mPa·s, at least 24,000 mPa·s, or higher. Combinations of the above-mentioned ranges are also possible. For example, in some embodiments, compositions comprising a biomaterial preparation described herein (e.g., a polymeric biomaterial in a precursor state or in a polymer network state such as, e.g., a viscous solution) may be characterized by a viscosity of 5 mPa·s to 10,000 mPa·s, or 10 mPa·s to 5000 mPa·s, or 5 mPa·s to 200 mPa·s, or 20 mPa·s to 100 mPa·s, or 5 mPa·s to 20 mPa·s, or 3 mPa·s to 15 mPa·s. In some embodiments, a biomaterial preparation described herein (e.g., a precursor state or a polymer network state such as, e.g., a viscous solution) can be a viscous solution with a viscosity similar to honey (e.g., with mPa·s and/or centipoise similar to honey, e.g., approximately 2,000 to 10,000 mPa·s). In some embodiments, a biomaterial preparation described herein (e.g., a precursor state or a polymer network state such as, e.g., a viscous solution) can be a viscous solution with a viscosity similar to natural syrup (e.g., a syrup from tree sap, a syrup from molasses, etc.) (e.g., with mPa·s and/or centipoise similar to natural syrups, e.g., approximately 15,000 to 20,000 mPa·s). In some embodiments, a biomaterial preparation described herein (e.g., a precursor state or a polymer network state such as, e.g., a viscous solution) can be a viscous solution with a viscosity similar to ketchup (e.g., tomato ketchup, e.g., with mPa·s and/or centipoise similar to ketchup, e.g., approximately 5,000 to 20,000 mPa·s). One skilled in the art reading the present disclosure will appreciate that, in some cases, viscosity of a composition comprising a biomaterial preparation described herein may be selected or adjusted based on, e.g., administration routes (e.g., injection vs. implantation), injection volume and/or time, and/or impact duration of immunomodulation. As will be also understood by one skilled in the art, viscosity of a biomaterial preparation depends on, e.g., temperature and concentration of the polymer in a testing sample. In some embodiments, viscosity of compositions comprising a biomaterial preparation described herein may be measured at 20° C., e.g., with a shear rate of 1000 s⁻¹.

In some embodiments, when compositions comprising a biomaterial preparation described herein is in a polymer network state, such a polymer network state may be characterized by a storage modulus of at least 100 Pa, at least 200 Pa, at least 300 Pa, at least 400 Pa, at least 500 Pa, at least 600 Pa, at least 700 Pa, at least 800 Pa, at least 900 Pa, at least 1000 Pa, at least 1100 Pa, at least 1200 Pa, at least 1300 Pa, at least 1400 Pa, at least 1500 Pa, at least 1600 Pa, at least 1700 Pa, at least 1800 Pa, at least 1900 Pa, at least 2000 Pa, at least 2100 Pa, at least 2200 Pa, at least 2300 Pa, at least 2400 Pa, at least 2500 Pa, at least 2600 Pa, at least 2700 Pa, at least 2800 Pa, at least 2900 Pa, at least 3000 Pa, at least 3500 Pa, at least 4000 Pa, at least 4500 Pa, at least 5000 Pa, at least 6000 Pa, at least 7000 Pa, at least 8000 Pa, at least 9000 Pa, or higher. In some embodiments, a biomaterial preparation in a polymer network may be characterized by a storage modulus of no more than 10 kPa, no more than 9 kPa, no more than 8 kPa, no more than 7 kPa, no more than 6 kPa, or lower. Combinations of the above-mentioned ranges are also possible. For example, in some embodiments, a biomaterial preparation in a polymer network may be characterized by a storage modulus of 100 Pa to 10 kPa, or 200 Pa to 5000 Pa, or 300 Pa to 2500 Pa, or 500 Pa to 2500 Pa or 100 Pa to 500 Pa. In some embodiments, a polymer network state of a provided biomaterial preparation may be characterized by a storage modulus of 1,000 Pa to 10,000 Pa, or 2,000 Pa to 10,000 Pa, or 3,000 Pa to 10,000 Pa, or 4,000 Pa to 10,000 Pa, or 5,000 Pa to 10,000, or 6,000 Pa to 10,000 Pa. One of those skilled in the art will appreciate that various rheological characterization methods (e.g., as described in Weng et al., “Rheological Characterization of in situ Crosslinkable Hydrogels Formulated from Oxidized Dextran and N-Carboxyethyl Chitosan” Biomacromolecules, 8: 1109-1115 (2007); the contents of which are incorporated herein in their entirety by reference for the purposes described herein) can be used to measure storage modulus of a material, and that, in some cases, storage modulus of a material may be measured with a rheometer and/or dynamic mechanical analysis (DMA). One of those skilled in the art will also appreciate that rheological characterization can vary with surrounding condition, e.g., temperature and/or pH.

Biomaterial preparations useful for compositions described herein are biocompatible. In some embodiments, biomaterial preparations useful for compositions described herein are biodegradable in vivo. In some embodiments, at least one polymer component in provided biomaterial preparations may be biodegradable in vivo. In some embodiments, at least one polymer component in provided biomaterial preparations may be resistant to biodegradation (e.g., via enzymatic and/or oxidative mechanisms). In some embodiments, at least one polymer component in provided biomaterial preparations may be chemically oxidized. Accordingly, in some embodiments, biomaterial preparations are able to be degraded, chemically and/or biologically, within a physiological environment, such as within a subject's body, e.g., at a target site of a subject. One of those skilled in the art will appreciate, reading the present disclosure, that degradation rates of provided biomaterial preparations may vary, e.g., based on selection of polymer component(s) and their material properties, and/or concentrations thereof (e.g., as described herein). For example, the half-life of provided biomaterial preparations (the time at which 50% of a biomaterial preparation is degraded into monomers and/or other non-polymeric moieties) may be on the order of days, weeks, months, or years. In some embodiments, biomaterial preparations described herein may be biologically degraded, e.g., by enzymatic activity or cellular machinery, for example, through exposure to a lysozyme (e.g., having relatively low pH), or by simple hydrolysis. In some cases, provided biomaterial preparations may be broken down into monomers (e.g., polymer monomers) and/or non-polymeric moieties that are non-toxic to cells. As will be understood by one of those skilled in the art, a provided biomaterial preparation has a longer residence time at a target site (e.g., a tumor resection site) upon administration if such a provided biomaterial preparation has a slower in vivo degradation rate.

In some embodiments, a provided biomaterial preparation is characterized in that, when assessed in vivo by administering to a target site (e.g., a tumor resection site) in a test subject (e.g., as described herein), at least 10% or more, including, e.g., at least 20%, at least 30%, at least 40%, at least 50%, at least 60%, at least 70%, at least 80%, at least 90%, or more, of such a provided biomaterial preparation in a polymer network state remains at the target site in vivo 2 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a provided biomaterial preparation in a polymer network state remains at a target site in vivo 2 days or more after the administration. Combinations of the above-mentioned are also possible. For example, in some embodiments, a provided biomaterial preparation is characterized in that, when assessed in vivo by administering to a target site (e.g., a tumor resection site) in a test subject (e.g., as described herein), 30%-80% or 40%-70% of such a provided biomaterial preparation in a polymer network state remains at the target site in vivo 2 days or more after the administration.

In some embodiments, a provided biomaterial preparation is characterized in that, when assessed in vivo by administering to a target site (e.g., a tumor resection site) in a test subject (e.g., as described herein), at least 10% or more, including, e.g., at least 20%, at least 30%, at least 40%, at least 50%, at least 60%, at least 70%, at least 80%, at least 90%, or more, of such a provided biomaterial preparation in a polymer network state remains at the target site in vivo 3 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a provided biomaterial preparation in a polymer network state remains at a target site in vivo 3 days or more after the administration. Combinations of the above-mentioned are also possible. For example, in some embodiments, a provided biomaterial preparation is characterized in that, when assessed in vivo by administering to a target site (e.g., a tumor resection site) in a test subject (e.g., as described herein), 30%-80% or 40%-70% of such a provided biomaterial preparation in a polymer network state remains at the target site in vivo 3 days or more after the administration.

In some embodiments, a provided biomaterial preparation is characterized in that, when assessed in vivo by administering to a target site (e.g., a tumor resection site) in a test subject (e.g., as described herein), at least 10% or more, including, e.g., at least 20%, at least 30%, at least 40%, at least 50%, at least 60%, at least 70%, at least 80%, at least 90%, or more, of such a provided biomaterial preparation in a polymer network state remains at the target site in vivo 5 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a provided biomaterial preparation in a polymer network state remains at a target site in vivo 5 days or more after the administration. Combinations of the above-mentioned are also possible. For example, in some embodiments, a provided biomaterial preparation is characterized in that, when assessed in vivo by administering to a target site (e.g., a tumor resection site) in a test subject (e.g., as described herein), 30%-80% or 40%-70% of such a provided biomaterial preparation in a polymer network state remains at the target site in vivo 5 days or more after the administration.

In some embodiments, a provided biomaterial preparation is characterized in that, when assessed in vivo by administering to a target site (e.g., a tumor resection site) in a test subject (e.g., as described herein), at least 10% or more, including, e.g., at least 20%, at least 30%, at least 40%, at least 50%, at least 60%, at least 70%, at least 80%, at least 90%, or more, of such a provided biomaterial preparation in a polymer network state remains at the target site in vivo 7 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a provided biomaterial preparation in a polymer network state remains at a target site in vivo 7 days or more after the administration. Combinations of the above-mentioned are also possible. For example, in some embodiments, a provided biomaterial preparation is characterized in that, when assessed in vivo by administering to a target site (e.g., a tumor resection site) in a test subject (e.g., as described herein), 30%-80% or 40%-70% of such a provided biomaterial preparation in a polymer network state remains at the target site in vivo 7 days or more after the administration.

In some embodiments, a provided biomaterial preparation is characterized in that, when assessed in vivo by administering to a target site (e.g., a tumor resection site) in a test subject (e.g., as described herein), at least 10% or more, including, e.g., at least 20%, at least 30%, at least 40%, at least 50%, at least 60%, at least 70%, at least 80%, at least 90%, or more, of such a provided biomaterial preparation in a polymer network state remains at the target site in vivo 14 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a provided biomaterial preparation in a polymer network state remains at a target site in vivo 14 days or more after the administration. Combinations of the above-mentioned are also possible. For example, in some embodiments, a provided biomaterial preparation is characterized in that, when assessed in vivo by administering to a target site (e.g., a tumor resection site) in a test subject (e.g., as described herein), 30%-80% or 40%-70% of such a provided biomaterial preparation in a polymer network state remains at the target site in vivo 14 days or more after the administration.

In some embodiments, a provided biomaterial preparation is characterized in that, when assessed in vivo by administering to a target site (e.g., a tumor resection site) in a test subject (e.g., as described herein), no more than 10% or less, including, e.g., no more than 9%, no more than 8%, no more than 7%, no more than 6%, no more than 5%, no more than 4%, no more than 3%, no more than 2%, no more than 1% or less, of such a provided biomaterial preparation in a polymer network state remains at the target site in vivo 10 days or more after the administration.

In certain embodiments, compositions described herein comprise a biomaterial preparation that forms a matrix or depot and a modulator of myeloid-derived suppressive cell function that is within the biomaterial preparation. In certain embodiments, a modulator of myeloid-derived suppressive cell function (e.g., a modulator of neutrophil function) is released from a biomaterial preparation after administration at a target site (e.g., a tumor resection site) by diffusion. For example, in certain embodiments, a polymer network state of a biomaterial preparation may be characterized in that, when tested in vitro by placing a composition comprising a biomaterial and a modulator of myeloid-derived suppressive cell function in PBS (pH 7.4), less than 100% (including, e.g., less than 95%, less than 90%, less than 85%, less than 80%, less than 70%, less than 50%, or lower) of the modulator of myeloid-derived suppressive cell function is released within 3 hours from the biomaterial preparation.

In certain embodiments, a polymer network state of a biomaterial preparation is characterized in that, when tested in vitro by placing a composition comprising a biomaterial and a modulator of myeloid-derived suppressive cell function in PBS (pH 7.4), at least 30% (including, e.g., at least 35%, at least 40%, at least 45%, at least 50%, at least 55%, at least 60%, at least 65%, or more) of the modulator of myeloid-derived suppressive cell function is released within 12 hours from the biomaterial preparation.

In certain embodiments, a polymer network state of a biomaterial preparation is characterized in that, when tested in vivo by administering a composition comprising a biomaterial and a modulator of myeloid-derived suppressive cell function at a mammary fat pad of a mouse subject, less than or equal to 60% (including, e.g., less than or equal to 50%, less than or equal to 40%, etc.) of the modulator of myeloid-derived suppressive cell function is released in vivo 8 hours after the administration.

In some embodiments, a composition provided herein is characterized in that a test animal group with spontaneous metastases having, at a tumor resection site, such a composition has a higher percent survival than that of a comparable test animal group having, at a tumor resection site, a biomaterial preparation without a modulator of myeloid-derived suppressive cell function, as assessed at 2 months after the administration. In some such embodiments, an increase in percent survival as observed in a test animal group with spontaneous metastases having, at a tumor resection site, a provided composition is at least 30% or more, including, at least 40%, at least 50%, at least 60%, at least 70%, at least 80%, at least 90%, or more, as compared to that of a comparable test animal group having, at a tumor resection site, a biomaterial preparation without a modulator of myeloid-derived suppressive cell function, as assessed at 2 months after the administration.

In some embodiments, a composition provided herein is characterized in that a test animal group with spontaneous metastases having, at a tumor resection site, such a composition has a higher percent survival than that of a comparable test animal group having, at a tumor resection site, a biomaterial preparation without a modulator of myeloid-derived suppressive cell function, as assessed at 3 months after the administration. In some such embodiments, an increase in percent survival as observed in a test animal group with spontaneous metastases having, at a tumor resection site, a provided composition is at least 10% or more, including, at least 20%, at least 30%, at least 40%, at least 50%, at least 60%, at least 70%, at least 80%, at least 90%, or more, as compared to that of a comparable test animal group having, at a tumor resection site, a biomaterial preparation without a modulator of myeloid-derived suppressive cell function, as assessed at 3 months after the administration.

In certain embodiments, biomaterial preparations described herein may form polymer networks with or without the addition of a cross-linking agent. In certain embodiments, a polymer network is crosslinked. Polymer networks (e.g., hydrogels) can be crosslinked using any methods known in the art, e.g., chemical crosslinking methods (e.g., by using a small-molecule cross-linker, which can be derived from a natural source or synthesized), polyelectrolyte crosslinking (e.g., mixing a polymer with a second polymer comprising an opposite charge), thermal-induced crosslinking, photo-induced crosslinking (e.g., using vinyl sulfone, methacrylate, acrylic acid), pH-induced crosslinking, and enzyme-catalyzed crosslinking. In some embodiments, one or more cross-linking methods described in Parhi, Adv Pharm Bull., Review 7(4): 515-530 (2017); which is incorporated herein by reference for the purposes described herein, can be used in forming a polymer network (e.g., a hydrogel).

(v) Optional Additional Therapeutic Agents

In some embodiments, a composition comprising a modulator of myeloid-derived suppressive cell function (e.g., a modulator of neutrophil function) may further comprise one or more additional therapeutic agents. For example, in some embodiments, such a therapeutic agent may be or comprise a chemotherapeutic agent. In some embodiments, such a therapeutic agent may be or comprise an immunomodulatory payload. In some embodiments, an immunomodulatory payload is or comprises a modulator of inflammation. As will be understood by appreciated by one of skilled in the art, inflammation may be immunostimulatory or immunosuppressive depending on the biological context. Accordingly, in some embodiments, an immunomodulatory payload is or comprises a modulator of immunostimulatory inflammation. In some embodiments, an immunomodulatory payload is or comprises a modulator of immunosuppressive inflammation. In some embodiments, an immunomodulatory payload is or comprises a modulator of innate immunity and/or adaptive immunity. In some such embodiments, a modulator of innate immunity and/or adaptive immunity is or comprises an agonist of innate immunity and/or adaptive immunity.

In some embodiments, an immunomodulatory payload is or comprises an immunomodulatory agent as described in International Patent Publication No. WO 2018/045058 (which includes, e.g., but not limited to examples of activators of innate immune response, activators of adaptive immune response, immunomodulatory cytokines, modulators of macrophage effector functions, etc.) and WO 2019/183216 (which includes, e.g., but not limited to inhibitors of immunosuppressive inflammation, e.g., mediated by a p38 mitogen-activated protein kinase (MAPK) pathway, etc.), the contents of each of which are incorporated herein by reference for purposes described herein. In some embodiments, an immunomodulatory payload is or comprises an activator of innate immune response, for example, in some embodiments, which may be or comprise a stimulator of interferon genes (STING) agonist, a Toll-like receptor (TLR) agonist, and/or an activator of innate immune response as described in International Patent Publication No. WO 2018/045058, the contents of which are incorporated herein by reference for purposes described herein. In some embodiments, an immunomodulatory payload is or comprises an inhibitor of immunosuppressive inflammation, for example, in some embodiments, which may be or comprise an inhibitor of immunosuppressive inflammation mediated by a p38 mitogen-activated protein kinase (MAPS) pathway, as described in International Patent Publication No. WO 2019/183216, the contents of which are incorporated herein by reference for purposes described herein.

### II. Exemplary Embodiments of Provided Compositions

In certain embodiments, a provided composition comprises a biomaterial preparation (e.g., comprising one or more polymers, one of which may be or comprise a poloxamer described herein) and an inhibitor of Bruton's tyrosine kinase (BTK).

In certain embodiments, a provided composition comprises a biomaterial preparation (e.g., comprising one or more polymers, one of which may be or comprise a poloxamer described herein) and Zanubrutinib.

In certain embodiments, a provided composition comprises a biomaterial preparation (e.g., comprising one or more polymers, one of which may be or comprise a poloxamer described herein) and an inhibitor of CSF-1.

In certain embodiments, a provided composition comprises a biomaterial preparation (e.g., comprising one or more polymers, one of which may be or comprise a poloxamer described herein) and an inhibitor of CSF1-R.

In certain embodiments, a provided composition comprises a biomaterial preparation (e.g., comprising one or more polymers, one of which may be or comprise a poloxamer described herein) and Edicotinib.

In certain embodiments, a provided composition comprises a biomaterial preparation (e.g., comprising one or more polymers, one of which may be or comprise a poloxamer described herein) and a promiscuous inhibitor of Tyrosine Kinases such as BCR/Abl, Src, c-Kit, and/or ephrin receptors.

In certain embodiments, a provided composition comprises a biomaterial preparation (e.g., comprising one or more polymers, one of which may be or comprise a poloxamer described herein) and dasatinib.

In certain embodiments, a provided composition may comprise a biomaterial preparation (e.g., comprising one or more polymers, one of which may be or comprise a poloxamer described herein) and an inhibitor of a COX-1 and/or COX-2 mediated signaling pathway.

In certain embodiments, a provided composition may comprise a biomaterial preparation (e.g., comprising one or more polymers, one of which may be or comprise a poloxamer described herein) and an inhibitor of COX-1.

In certain embodiments, a provided composition may comprise a biomaterial preparation (e.g., comprising one or more polymers, one of which may be or comprise a poloxamer described herein) and Ketorolac.

In certain embodiments, a provided composition may comprise a biomaterial preparation (e.g., comprising one or more polymers, one of which may be or comprise a poloxamer described herein) and Lornoxicam.

In certain embodiments, a provided composition may comprise a biomaterial preparation (e.g., comprising one or more polymers, one of which may be or comprise a poloxamer described herein) and a Phosphodiesterase type 5 (PDE5) inhibitor.

In certain embodiments, a provided composition may comprise a biomaterial preparation (e.g., comprising one or more polymers, one of which may be or comprise a poloxamer described herein) and Sildenafil.

In certain embodiments, a provided composition may comprise a biomaterial preparation (e.g., comprising one or more polymers, one of which may be or comprise a poloxamer described herein) and an Inhibitor of apoptosis (IAP) inhibitor.

In certain embodiments, a provided composition may comprise a biomaterial preparation (e.g., comprising one or more polymers, one of which may be or comprise a poloxamer described herein) and Birinapant.

In certain embodiments, a provided composition comprises a biomaterial preparation (e.g., comprising one or more polymers, one of which may be or comprise a poloxamer described herein) and an inhibitor of Triggering receptor expressed on myeloid cells 1 (TREM-1).

In certain embodiments, a provided composition comprises a biomaterial preparation (e.g., comprising one or more polymers, one of which may be or comprise a poloxamer described herein) and anti-TREM-1 (PY159).

In certain embodiments, a provided composition comprises a biomaterial preparation (e.g., comprising one or more polymers, one of which may be or comprise a poloxamer described herein) and an inhibitor of Triggering receptor expressed on myeloid cells 1 (TREM-2).

In certain embodiments, a provided composition comprises a biomaterial preparation (e.g., comprising one or more polymers, one of which may be or comprise a poloxamer described herein) and anti-TREM-2 (PY314).

In certain embodiments, a provided composition comprises a biomaterial preparation (e.g., comprising one or more polymers, one of which may be or comprise a poloxamer described herein) and an inhibitor of CD47.

In certain embodiments, a provided composition may comprise a biomaterial preparation (e.g., comprising one or more polymers, one of which may be or comprise a poloxamer described herein) and Hu5F9-G4.

In certain embodiments, a provided composition comprises a biomaterial preparation (e.g., comprising one or more polymers, one of which may be or comprise a poloxamer described herein) and an inhibitor of matrix metallopeptidases.

In certain embodiments, a provided composition comprises a biomaterial preparation (e.g., comprising one or more polymers, one of which may be or comprise in some embodiments a poloxamer described herein) and JNJ0966, BMS-P5, GSK199, GSK484, aprotinin, Hu5F9-G4, and/or any combination thereof.

In certain embodiments, a provided composition comprises a biomaterial preparation (e.g., comprising one or more polymers, one of which may be or comprise a poloxamer described herein) and an inhibitor of matrix metallopeptidase 9.

In certain embodiments, a provided composition comprises a biomaterial preparation (e.g., comprising one or more polymers, one of which may be or comprise a poloxamer described herein) and JNJ0966.

In certain embodiments, a provided composition comprises a biomaterial preparation (e.g., comprising one or more polymers, one of which may be or comprise a poloxamer described herein) and an inhibitor of elastase.

In certain embodiments, a provided composition comprises a biomaterial preparation (e.g., comprising one or more polymers, one of which may be or comprise a poloxamer described herein) and aprotinin.

In certain embodiments, a provided composition comprises a biomaterial preparation (e.g., comprising one or more polymers, one of which may be or comprise a poloxamer described herein) and an inhibitor of NETosis.

In certain embodiments, a provided composition comprises a biomaterial preparation (e.g., comprising one or more polymers, one of which may be or comprise a poloxamer described herein) and BMS-P5, GSK199, GSK484, and/or any combination thereof.

In certain embodiments, a provided composition comprises a biomaterial preparation (e.g., comprising one or more polymers, one of which may be or comprise a poloxamer described herein) and a DNase (e.g., DNase I, and/or DNase I-like 3).

In certain embodiments, a provided composition may comprise a biomaterial preparation (e.g., comprising one or more polymers, one of which may be or comprise a poloxamer described herein) and an inhibitor of VEGF, VEGFR, VEGFR1, VEGFR2, VEGFR3, and/or any combination thereof.

In certain embodiments, a provided composition comprises a biomaterial preparation (e.g., comprising one or more polymers, one of which may be or comprise a poloxamer described herein) and an inhibitor of HGF and/or HGFR signaling.

In certain embodiments, a provided composition comprises a biomaterial preparation (e.g., comprising one or more polymers, one of which may be or comprise a poloxamer described herein) and an inhibitor of HGFR.

In certain embodiments, a provided composition comprises a biomaterial preparation (e.g., comprising one or more polymers, one of which may be or comprise a poloxamer described herein) and metformin.

In certain embodiments, a provided composition may comprise a biomaterial preparation (e.g., comprising one or more polymers, one of which may be or comprise a poloxamer described herein) and an inhibitor of TGFβ, TGF-βR, TGF-βR1, TGF-βR2, TGF-βR3, and/or any combination thereof.

In certain embodiments, a provided composition may comprise a biomaterial preparation (e.g., comprising one or more polymers, one of which may be or comprise a poloxamer described herein) and Galunisertib.

In certain embodiments, a provided composition may comprise a biomaterial preparation (e.g., comprising one or more polymers, one of which may be or comprise a poloxamer described herein) and an inhibitor of arginase.

In certain embodiments, a provided composition may comprise a biomaterial preparation (e.g., comprising one or more polymers, one of which may be or comprise a poloxamer described herein) and an inhibitor of LTB4.

In certain embodiments, a provided composition may comprise a biomaterial preparation (e.g., comprising one or more polymers, one of which may be or comprise a poloxamer described herein) and an activator of a specialized pro-resolving mediator.

In certain embodiments, a provided composition may comprise a biomaterial preparation (e.g., comprising one or more polymers, one of which may be or comprise a poloxamer described herein) and resolvin.

In certain embodiments, a provided composition may comprise a biomaterial preparation (e.g., comprising one or more polymers, one of which may be or comprise a poloxamer described herein) and RvD2.

In certain embodiments, a provided composition may comprise a biomaterial preparation (e.g., comprising one or more polymers, one of which may be or comprise a poloxamer described herein) and LXA4.

In certain embodiments, a provided composition may comprise a biomaterial preparation (e.g., comprising one or more polymers, one of which may be or comprise a poloxamer described herein) and an inhibitor of CXCR1 and/or CXCR2.

In certain embodiments, a provided composition may comprise a biomaterial preparation (e.g., comprising one or more polymers, one of which may be or comprise a poloxamer described herein) and Reparixin.

In certain embodiments, a provided composition may comprise a biomaterial preparation (e.g., comprising one or more polymers, one of which may be or comprise a poloxamer described herein) and a CCR2 inhibitor.

In certain embodiments, a provided composition may comprise a biomaterial preparation (e.g., comprising one or more polymers, one of which may be or comprise a poloxamer described herein) and BMS-813160, BMS CCR2 22, MK-0812, CCX872, PF-04136309, and/or any combination thereof.

In certain embodiments, a provided composition may comprise a biomaterial preparation (e.g., comprising one or more polymers, one of which may be or comprise a poloxamer described herein) and a CCL2 inhibitor.

In certain embodiments, a provided composition may comprise a biomaterial preparation (e.g., comprising one or more polymers, one of which may be or comprise a poloxamer described herein) and Bindarit.

In certain embodiments, a provided composition may comprise a biomaterial preparation (e.g., comprising one or more polymers, one of which may be or comprise a poloxamer described herein) and an inhibitor of CCL2, CCL3, CCL4, CCL5, CCL8, and/or any combination thereof.

In certain embodiments, a provided composition may comprise a biomaterial preparation (e.g., comprising one or more polymers, one of which may be or comprise a poloxamer described herein) and an inhibitor of CCR1, CCR2, CCR3, CCR4, CCR5 CCR8, and/or any combination thereof.

In certain embodiments, a provided composition may comprise a biomaterial preparation (e.g., comprising one or more polymers, one of which may be or comprise a poloxamer described herein) and an inhibitor of CCL2/CCR2 signaling, and/or CCL2/CCR4 signaling.

In certain embodiments, a provided composition may comprise a biomaterial preparation (e.g., comprising one or more polymers, one of which may be or comprise a poloxamer described herein) and an inhibitor of CCL3/CCR1 signaling, CCL3/CCR4 signaling, CCL3/CCR5 signaling, and/or any combination thereof.

In certain embodiments, a provided composition may comprise a biomaterial preparation (e.g., comprising one or more polymers, one of which may be or comprise a poloxamer described herein) and an inhibitor of CCL4/CCR1 signaling, and/or CCL4/CCR5 signaling.

In certain embodiments, a provided composition may comprise a biomaterial preparation (e.g., comprising one or more polymers, one of which may be or comprise a poloxamer described herein) and an inhibitor of CCL5/CCR1 signaling, CCL5/CCR3 signaling, CCL5/CCR4 signaling, CCL5/CCR5 signaling, and/or any combination thereof.

In certain embodiments, a provided composition may comprise a biomaterial preparation (e.g., comprising one or more polymers, one of which may be or comprise a poloxamer described herein) and an inhibitor of CCL8/CCR2 signaling, CCL8/CCR3 signaling, CCL8/CCR5 signaling, and/or any combination thereof.

In certain embodiments, a provided composition may comprise a biomaterial preparation (e.g., comprising one or more polymers, one of which may be or comprise a poloxamer described herein) and an inhibitor of CXCR4 and/or CXCL12.

In certain embodiments, a provided composition may comprise a biomaterial preparation (e.g., comprising one or more polymers, one of which may be or comprise a poloxamer described herein) and plerixafor.

In certain embodiments, a provided composition comprises a biomaterial preparation (e.g., comprising one or more polymers, one of which may be or comprise a poloxamer described herein) and an inhibitor of Macrophage Migration Inhibitory Factor (MIF).

In certain embodiments, a provided composition comprises a biomaterial preparation (e.g., comprising one or more polymers, one of which may be or comprise a poloxamer described herein) and an inhibitor of CD74.

In certain embodiments, a provided composition comprises a biomaterial preparation (e.g., comprising one or more polymers, one of which may be or comprise a poloxamer described herein) and 4-IPP.

In certain embodiments, a provided composition comprises a biomaterial preparation (e.g., comprising one or more polymers, one of which may be or comprise a poloxamer described herein) and an anti-CD74 monoclonal antibody.

In certain embodiments, a provided composition may comprise a biomaterial preparation (e.g., comprising one or more polymers, one of which may be or comprise a poloxamer described herein) and an inhibitor of Adenosine A2A receptor and/or A2B receptor.

In certain embodiments, a provided composition may comprise a biomaterial preparation (e.g., comprising one or more polymers, one of which may be or comprise a poloxamer described herein) and theophylline.

In certain embodiments, a provided composition may comprise a biomaterial preparation (e.g., comprising one or more polymers, one of which may be or comprise a poloxamer described herein) and etrumadenant (AB928).

In certain embodiments, a provided composition may comprise a biomaterial preparation (e.g., comprising one or more polymers, one of which may be or comprise a poloxamer described herein) and istradefylline, AZD4635, MK-3814, and/or any combination thereof.

In certain embodiments, a provided composition may comprise a biomaterial preparation (e.g., comprising one or more polymers, one of which may be or comprise a poloxamer described herein) and alloxazine.

In certain embodiments, a provided composition may comprise a biomaterial preparation (e.g., comprising one or more polymers, one of which may be or comprise a poloxamer described herein) and an inhibitor of CD39.

In certain embodiments, a provided composition may comprise a biomaterial preparation (e.g., comprising one or more polymers, one of which may be or comprise a poloxamer described herein) and an inhibitor of CD73.

In certain embodiments, a provided composition may comprise a biomaterial preparation (e.g., comprising one or more polymers, one of which may be or comprise a poloxamer described herein) and AB680, BMS-986179, MEDI9447, and/or any combination thereof.

In certain embodiments, a provided composition comprises a biomaterial preparation (e.g., comprising one or more polymers, one of which may be or comprise a poloxamer described herein) and an inhibitor of P2RX7 signaling.

In certain embodiments, a provided composition comprises a biomaterial preparation (e.g., comprising one or more polymers, one of which may be or comprise a poloxamer described herein) and GSK1482160, JNJ-5417544, JNJ-479655, and/or any combination thereof.

In certain embodiments, a provided composition comprises a biomaterial preparation (e.g., comprising one or more polymers, one of which may be or comprise a poloxamer described herein) and an inhibitor of ADAR1.

In certain embodiments, a provided composition comprises a biomaterial preparation (e.g., comprising one or more polymers, one of which may be or comprise a poloxamer described herein) and 8-azaadenosine.

In certain embodiments, a provided composition comprises a biomaterial preparation (e.g., comprising one or more polymers, one of which may be or comprise a poloxamer described herein) and a modulator of angiopoietin signaling.

In certain embodiments, a provided composition comprises a biomaterial preparation (e.g., comprising one or more polymers, one of which may be or comprise a poloxamer described herein) and an inhibitor of Angiopoietin-2.

In certain embodiments, a provided composition comprises a biomaterial preparation (e.g., comprising one or more polymers, one of which may be or comprise a poloxamer described herein) and an inhibitor of Cathepsin G.

In certain embodiments, a provided composition comprises a biomaterial preparation (e.g., comprising one or more polymers, one of which may be or comprise a poloxamer described herein) and an inhibitor of IL-34 signaling.

In certain embodiments, a provided composition comprises a biomaterial preparation (e.g., comprising one or more polymers, one of which may be or comprise a poloxamer described herein) and an inhibitor of P2RX4.

In certain embodiments, a provided composition comprises a biomaterial preparation (e.g., comprising one or more polymers, one of which may be or comprise a poloxamer described herein) and an inhibitor of IL-1α signaling.

In certain embodiments, a provided composition comprises a biomaterial preparation (e.g., comprising one or more polymers, one of which may be or comprise a poloxamer described herein) and an inhibitor of a dopaminergic receptor and/or an antipsychotic agent.

In certain embodiments, a provided composition comprises a biomaterial preparation (e.g., comprising one or more polymers, one of which may be or comprise a poloxamer described herein) and prochlorperazine.

In certain embodiments, a provided composition comprises a biomaterial preparation (e.g., comprising one or more polymers, one of which may be or comprise a poloxamer described herein) and an agent that causes neutropenia.

In certain embodiments, a provided composition comprises a biomaterial preparation (e.g., comprising one or more polymers, one of which may be or comprise a poloxamer described herein) and an inhibitor of a TAM family receptor tyrosine kinase related signaling pathway.

In certain embodiments, a provided composition comprises a biomaterial preparation (e.g., comprising one or more polymers, one of which may be or comprise a poloxamer described herein) and cabozantinib, merestinib, BMS-777607, S49076, ONO-7475, RXDX-106, LDC1267, sitravatinib, UNC2025, and/or any combination thereof.

In certain embodiments, a provided composition comprises a biomaterial preparation (e.g., comprising one or more polymers, one of which may be or comprise a poloxamer described herein) and LDC1267.

In certain embodiments, a provided composition comprises a biomaterial preparation (e.g., comprising one or more polymers, one of which may be or comprise a poloxamer described herein) and sitravatinib.

In certain embodiments, a provided composition comprises a biomaterial preparation (e.g., comprising one or more polymers, one of which may be or comprise a poloxamer described herein) and an inhibitor of LAIR-1.

In certain embodiments, a provided composition comprises a biomaterial preparation (e.g., comprising one or more polymerase, one of which may be or comprise a poloxamer described herein) and a modulator of a LILR associated signaling pathway.

In certain embodiments, a provided composition comprises a biomaterial preparation (e.g., comprising one or more polymerase, one of which may be or comprise a poloxamer described herein) and a modulator of ILT2.

In certain embodiments, a provided composition comprises a biomaterial preparation (e.g., comprising one or more polymerase, one of which may be or comprise a poloxamer described herein) and an anti-ILT2 antibody.

In certain embodiments, a provided composition comprises a biomaterial preparation (e.g., comprising one or more polymerase, one of which may be or comprise a poloxamer described herein) and a modulator of ILT3.

In certain embodiments, a provided composition comprises a biomaterial preparation (e.g., comprising one or more polymerase, one of which may be or comprise a poloxamer described herein) and an anti-ILT3 antibody.

In certain embodiments, a provided composition comprises a biomaterial preparation (e.g., comprising one or more polymerase, one of which may be or comprise a poloxamer described herein) and a modulator of ILT4.

In certain embodiments, a provided composition comprises a biomaterial preparation (e.g., comprising one or more polymerase, one of which may be or comprise a poloxamer described herein) and an anti-ILT4 antibody.

In certain embodiments, a provided composition comprises a biomaterial preparation (e.g., comprising one or more polymers, one of which may be or comprise a poloxamer described herein) and an inhibitor of a c-Kit related signaling pathway.

In certain embodiments, a provided composition comprises a biomaterial preparation (e.g., comprising one or more polymers, one of which may be or comprise a poloxamer described herein) and an inhibitor of a MET related signaling pathway.

In certain embodiments, a provided composition comprises a biomaterial preparation (e.g., comprising one or more polymers, one of which may be or comprise a poloxamer described herein) and an inhibitor of IL-4R signaling.

In certain embodiments, a provided composition comprises a biomaterial preparation (e.g., comprising one or more polymers, one of which may be or comprise a poloxamer described herein) and vorinostat.

In certain embodiments, a provided composition comprises a biomaterial preparation (e.g., comprising one or more polymers, one of which may be or comprise a poloxamer described herein) and an inhibitor of MAO-A.

In certain embodiments, a provided composition comprises a biomaterial preparation (e.g., comprising one or more polymers, one of which may be or comprise a poloxamer described herein) and phenelzine, clorgyline, mocolobemide, pirlindole, and/or any combination thereof.

In certain embodiments, a provided composition comprises a biomaterial preparation (e.g., comprising one or more polymers, one of which may be or comprise a poloxamer described herein) and an inhibitor of C₅a and/or C₅aR.

In certain embodiments, a provided composition comprises a biomaterial preparation (e.g., comprising one or more polymers, one of which may be or comprise a poloxamer described herein) and a corticosteroid.

In certain embodiments, a provided composition comprises a biomaterial preparation (e.g., comprising one or more polymers, one of which may be or comprise a poloxamer described herein) and a glucocorticoid.

In certain embodiments, a provided composition comprises a biomaterial preparation (e.g., comprising one or more polymers, one of which may be or comprise a poloxamer described herein) and dexamethasone.

In certain embodiments, a provided composition comprises a biomaterial preparation (e.g., comprising one or more polymers, one of which may be or comprise a poloxamer described herein) and an activator of glutamate-gated chloride channels and/or a positive allosteric effector of P2RX4, P2RX7, 07 nAChR, and/or any combination thereof.

In certain embodiments, a provided composition comprises a biomaterial preparation (e.g., comprising one or more polymers, one of which may be or comprise a poloxamer described herein) and ivermectin.

In certain embodiments, a provided composition comprises a biomaterial preparation (e.g., comprising one or more polymers, one of which may be or comprise a poloxamer described herein) and a beta-adrenergic receptor antagonist.

In certain embodiments, a provided composition comprises a biomaterial preparation (e.g., comprising one or more polymers, one of which may be or comprise a poloxamer described herein) and propranolol.

In certain embodiments, a provided composition comprises a biomaterial preparation (e.g., comprising one or more polymers, one of which may be or comprise a poloxamer described herein) and timolol.

In certain embodiments, a provided composition comprises a biomaterial preparation (e.g., comprising one or more polymers, one of which may be or comprise a poloxamer described herein) and an inhibitor of the renin-angiotensin system.

In certain embodiments, a provided composition comprises a biomaterial preparation (e.g., comprising one or more polymers, one of which may be or comprise a poloxamer described herein) and an ACE inhibitor.

In certain embodiments, a provided composition comprises a biomaterial preparation (e.g., comprising one or more polymers, one of which may be or comprise a poloxamer described herein) and an angiotensin II receptor inhibitor.

In certain embodiments, a provided composition comprises a biomaterial preparation (e.g., comprising one or more polymers, one of which may be or comprise a poloxamer described herein) and valsartan.

### III. Pharmaceutical Compositions

In some embodiments, a provided composition can be formulated in accordance with routine procedures as a pharmaceutical composition for administration to a subject in need thereof (e.g., as described herein). In some embodiments, such a pharmaceutical composition can include a pharmaceutically acceptable carrier or excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, M D, 2006; incorporated herein by reference) discloses various excipients used in formulating pharmaceutical compositions and known techniques for the preparation thereof. Suitable pharmaceutically acceptable carriers include but are not limited to water, salt solutions (e.g., NaCl), saline, buffered saline, glycerol, sugars such as mannitol, lactose, trehalose, sucrose, or others, dextrose, fatty acid esters, etc., as well as combinations thereof.

A pharmaceutical composition can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like), which do not deleteriously react with the active compounds or interfere with their activity. In some embodiments, a pharmaceutical composition can be sterile. A suitable pharmaceutical composition, if desired, can also contain minor amounts of wetting or emulsifying agents, or pH buffering agents. A pharmaceutical composition can be a liquid solution, suspension, or emulsion.

A pharmaceutical composition can be formulated in accordance with the routine procedures as a pharmaceutical composition adapted for administration to human beings. The formulation of a pharmaceutical composition should suit the mode of administration. For example, in some embodiments, a pharmaceutical composition for injection may typically comprise sterile isotonic aqueous buffer. Where necessary, a pharmaceutical composition may also include a local anesthetic to ease pain at a site of injection. In some embodiments, components of a pharmaceutical composition (e.g., as described herein) are supplied separately or mixed together in a single-use form, for example, as a dry lyophilized powder or water free concentrate in a hermetically sealed container such as an ampule or sachet or in a sterile syringe indicating the quantity of a composition comprising a biomaterial preparation and a modulator of myeloid-derived suppressive cell function (e.g., ones described herein). Where a pharmaceutical composition is to be administered by injection, in some embodiments, a dry lyophilized powder composition comprising a biomaterial preparation and a modulator of myeloid-derived suppressive cell function (e.g., ones described herein) can be reconstituted with an aqueous buffered solution and then injected to a target site in a subject in need thereof. In some embodiments, a liquid composition comprising a biomaterial preparation and a modulator of myeloid-derived suppressive cell function (e.g., ones described herein) can be provided in a syringe for administration by injection and/or by a robotic surgical system (e.g., a da Vinci System).

In some embodiments, a liquid composition comprising a biomaterial preparation and a modulator of myeloid-derived suppressive cell function (e.g., ones described herein) can be provided in a syringe for administration with or without a needle, cannula, or trocar.

In some embodiments, a liquid composition comprising a biomaterial preparation and a modulator of myeloid-derived suppressive cell function (e.g., ones described herein) can be administered by spraying.

In some embodiments, administration of a liquid composition comprising a biomaterial preparation and a modulator of myeloid-derived suppressive cell function (e.g., ones described herein) can be gas assisted for use in minimally invasive surgery.

In some embodiments, administration of a liquid composition comprising a biomaterial preparation and a modulator of myeloid-derived suppressive cell function (e.g., ones described herein) can be achieved by using a multi-barrel syringe, with each barrel containing a separate polymer component preparation, the multiple of which are combined upon depression of the shared plunger.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts or cells in vitro or ex vivo. Modification of pharmaceutical compositions suitable for administration to humans in order to render the compositions suitable for administration to various animals or cells in vitro or ex vivo is well understood, and the ordinarily skilled practitioner, e.g., a veterinary pharmacologist, can design and/or perform such modification with merely ordinary, if any, experimentation.

Formulations of the pharmaceutical compositions described herein may be prepared by any method known or hereafter developed in the art of pharmacology. For example, such preparatory methods include step of bringing components of a provided composition comprising a biomaterial preparation and a modulator of myeloid-derived suppressive cell function (e.g., ones described herein), into association with a diluent or another excipient and/or one or more other accessory ingredients and then, if necessary and/or desirable, shaping and/or packaging the product into a desired single-use unit or multi-use units. Alternatively, such preparatory methods may also include a step of pre-forming a composition comprising a biomaterial preparation and a modulator of myeloid-derived suppressive cell function (e.g., ones described herein) into a polymer network state (e.g., a hydrogel), prior to shaping and/or packaging the product into a desired single-use units or multi-use units.

A pharmaceutical composition in accordance with the present disclosure may be prepared, packaged, and/or sold in bulk, as a single-use unit, and/or as a plurality of single-use units. As used herein, a “single-use unit” is a discrete amount of a pharmaceutical composition described herein. For example, a single-use unit of a pharmaceutical composition comprises a predetermined amount of a composition described herein, which in some embodiments can be or comprise a pre-formed polymer network of a biomaterial preparation (e.g., ones described herein) with a modulator of myeloid-derived suppressive cell function (e.g., ones described herein), or in some embodiments can be or comprise a liquid or a colloidal mixture of individual components of a composition (e.g., ones described herein).

The relative amount of individual components of a provided composition and, optionally, any additional agents in pharmaceutical compositions described herein, e.g., a pharmaceutically acceptable excipient and/or any additional ingredients, can vary, depending upon, e.g., desired material properties of a polymer biomaterial, size of target site, injection volume, physical and medical condition of a subject to be treated, and/or types of cancer, and may also further depend upon the route by which such a pharmaceutical composition is to be administered. In some embodiments, a modulator of myeloid-derived suppressive cell function (e.g., as described herein) is provided in an effective amount in a pharmaceutical composition to provide a desired therapeutic effect (e.g., but not limited to inducing anti-tumor immunity in at least one or more aspects, e.g., inhibiting recruitment and/or survival and/or proliferation of neutrophils and/or modulating neutrophil-associated effector function). In some embodiments, a modulator of myeloid-derived suppressive cell function (e.g., as described herein) is provided in an effective amount in a pharmaceutical composition for treatment of cancer. In some embodiments, a modulator of myeloid-derived suppressive cell function (e.g., as described herein) is provided in an effective amount in a pharmaceutical composition to inhibit or reduce risk or incidence of tumor recurrence and/or metastasis. In certain embodiments, the effective amount is a therapeutically effective amount of a biomaterial preparation and a modulator of myeloid-derived suppressive cell function (e.g., as described herein). In certain embodiments, the effective amount is a prophylactically effective amount of a biomaterial preparation and a modulator of myeloid-derived suppressive cell function (e.g., as described herein).

In certain embodiments, pharmaceutical compositions do not include cells. In certain embodiments, pharmaceutical compositions do not include adoptively transferred cells. In certain embodiments, pharmaceutical compositions do not include T cells. In certain embodiments, pharmaceutical compositions do not include tumor antigens. In certain embodiments, pharmaceutical compositions do not include tumor antigens loaded ex vivo.

In certain embodiments, a pharmaceutical composition is in liquid form (e.g., a solution or a colloid). In certain embodiments, a pharmaceutical composition is in a solid form (e.g., a gel form). In certain embodiments, the transition from a liquid form to a solid form may occur outside a subject's body upon sufficient crosslinking such that the resulting material has a storage modulus consistent with a solid form that allows it to be physically manipulated and implanted in a surgical procedure. Accordingly, in some embodiments, a solid form may be amenable for carrying out an intended use of the present disclosure (e.g., surgical implantation). In certain embodiments, the transition from a liquid form to a solid form may occur upon thermal crosslinking in situ (e.g., inside a body of a subject) such that the resulting material has a storage modulus consistent with a solid form. In certain embodiments, a pharmaceutical composition is a suspension.

### IV. Therapeutic Uses

Technologies provided herein are useful for treatment of cancer. In some embodiments, technologies provided herein are useful to delay the onset of, slow the progression of, or ameliorate one or more symptoms of cancer. In some embodiments, technologies provided herein are useful to reduce or inhibit primary tumor regrowth. In some embodiments, technologies provided herein are useful to reduce or inhibit incidence of tumor recurrence and/or metastasis. In some embodiments, technologies provided herein are useful for inducing anti-tumor immunity.

Accordingly, some aspects provided herein relate to methods of administering to a target site in a subject in need thereof a composition comprising a biomaterial preparation described herein. In some embodiments, a subject receiving such a composition may be undergoing or may have undergone tumor removal (e.g., by surgical tumor resection). In some embodiments, a subject receiving such a composition may have tumor relapse and/or metastasis. In some such embodiments, a method comprises intraoperative administration of a composition comprising a biomaterial preparation described herein at a tumor resection site of a subject. In some embodiments, such a provided composition utilized in methods of the present disclosure may be formulated as a pharmaceutical composition described herein.

In certain embodiments, a method provided herein comprises administering a provided composition to a target site in a subject in need thereof after removal of tumor, for example, after removal of greater than or equal to 50% or higher, by weight, of the subject's tumor, including, e.g., greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 96%, greater than or equal to 97%, greater than or equal to 98%, or greater than or equal to 99%, by weight, of the subject's tumor. In certain embodiments, a method provided herein comprises administering a provided composition to a target site in a subject in need thereof after removal of greater than or equal to 50% or higher, by volume, of the subject's tumor, including, e.g., greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 96%, greater than or equal to 97%, greater than or equal to 98%, or greater than or equal to 99%, by volume, of the subject's tumor. In some embodiments, a method provided herein comprises performing a tumor resection to remove a subject's tumor, prior to administration of a provided composition.

In some embodiments, a composition described and/or utilized herein is administered to a target site in a tumor resection subject immediately after the subject's tumor has been removed by surgical tumor resection. In some embodiments, a composition described and/or utilized herein is intraoperatively administered to a target site in a tumor section subject. In some embodiments, a composition described and/or utilized herein is postoperatively administered to a target site in a tumor resection subject within 24 hours or less, including, e.g., within 18 hours, within 12 hours, within 6 hours, within 3 hours, within 2 hours, within 1 hour, within 30 mins, or less, after the subject's tumor has been removed by surgical tumor resection. In some embodiments, a composition described and/or utilized herein is postoperatively administered one or more times to one or more target sites at one or more time points within 12 months or less from a surgical intervention, including e.g., within 11 months, within 10 months, within 9 months, within 8 months, within 7 months, within 6 months, within 5 months, within 4 months, within 3 months, within 2 months, or within 1 months of a surgical intervention. In some embodiments, a composition described and/or utilized herein is postoperatively administered one or more times to one or more target sites at one or more time points within 31 days, including e.g., within 30 days, within 29 days, within 28 days, within 27 days, within 26 days, within 25 days, within 24 days, within 23 days, within 22 days, within 21 days, within 20 days, within 19 days, within 18 days, within 17 days, within 16 days, within 15 days, within 14 days, within 13 days, within 12 days, within 11 days, within 10 days, within 9 days, within 8 days, within 7 days, within 6 days, within 5 days, within 4 days, within 3 days, within 2 days, or within 1 day of a surgical intervention.

In some embodiments, a target site for administration is or comprises a tumor resection site. In some embodiments, such a tumor resection site may be characterized by absence of gross residual tumor antigen. In some embodiments, such a tumor resection site may be characterized by a negative resection margin (i.e., no cancer cells seen microscopically at the resection margin, e.g., based on histological assessment of tissues surrounding the tumor resection site). In some embodiments, such a tumor resection site may be characterized by a positive resection margin (i.e., cancer cells are seen microscopically at the resection margin, e.g., based on histological assessment of tissues surrounding the tumor resection site). In some embodiments, such a tumor resection site may be characterized by presence of gross residual tumor antigen. In some embodiments, a target site for administration is or comprises a site in close proximity to a tumor resection site. In some embodiments, a target site for administration is or comprises a site within 4 inches (including, e.g., within 3.5 inches, within 3 inches, within 2.5 inches, within 2 inches, within 1.5 inches, within 1 inches, within 0.5 inches, within 0.4 inches, within 0.3 inches, within 0.2 inches, within 0.1 inches or less) of a tumor resection site. In some embodiments, a target site for administration is or comprises a site within 10 centimeters (including, e.g., within 9 centimeters, within 8 centimeters, within 7 centimeters, within 6 centimeters, within 5 centimeters, within 4 centimeters, within 3 centimeters, within 2 centimeters, within 1 centimeter, within 0.5 centimeters or less) of a tumor resection site. In some embodiments, a target site for administration is or comprises a sentinel lymph node. In some embodiments, a target site for administration is or comprises a draining lymph node.

As will be understood by one of ordinary skill in the art, compositions that are useful in accordance with the present disclosure can be administered to a target site in subjects in need thereof using appropriate delivery approaches known in the art. For example, in some embodiments, provided technologies can be amenable for administration by injection. In some embodiments, provided technologies can be amenable for administration by minimally invasive surgery (MIS), e.g., robot-assisted MIS, robotic surgery, and/or laparoscopic surgery, which, for example, typically involve one or more small incisions. In some embodiments, provided technologies can be amenable for administration in the context of accessible and/or cutaneous excisions. In some embodiments, provided technologies can be amenable for administration (e.g., by injection) intraoperatively as part of minimally invasive procedure, e.g., minimally invasive surgery (MIS), e.g., robot-assisted MIS, robotic surgery, and/or laparoscopic surgery, and/or procedure that involves one or more accessible and/or cutaneous excisions. In some embodiments, provided technologies can be amenable for administration (e.g., by injection) involving a robotic surgical system (e.g., a da Vinci System), e.g., in some embodiments for minimally invasive administration. For example, in some embodiments, a composition that may be useful for injection and/or in the context of minimally invasive procedure, e.g., minimally invasive surgery (MIS), e.g., robot-assisted MIS, robotic surgery, and/or laparoscopic surgery and/or procedure that involves one or more accessible and/or cutaneous excisions, is liquid and a biomaterial preparation provided in such a composition is or comprises a polymer solution (e.g., a viscous polymer solution), which upon injection to a target site (e.g., a tumor resection site) in a subject, it transitions from a liquid solution state to a polymer network state (e.g., a hydrogel), which in some embodiments, such a transition is triggered by exposure to the body temperature of the subject. In some embodiments, a biomaterial preparation in a pre-formed polymer network biomaterial that is compressible without adversely impact its structural integrity can be injected, for example, by a minimally invasive procedure, e.g., minimally invasive surgery (MIS), e.g., robot-assisted MIS, robotic surgery, and/or laparoscopic surgery and/or procedure.

In some embodiments, technologies provided herein can be amenable for administration by implantation. For example, in some embodiments, a biomaterial preparation provided in a composition in accordance with the present disclosure is a pre-formed polymer network biomaterial. An exemplary polymer network biomaterial is or comprises a hydrogel. For example, in some embodiments, a provided composition may be administered by surgical implantation to a tumor resection site (e.g., void volume resulting from tumor resection). In some embodiments, a provided composition may be administered by surgical implantation to a tumor resection site and affixed with a bioadhesive. In some embodiments, administration may be performed intraoperatively (i.e., immediately after tumor resection).

In some embodiments, the amount of a biomaterial preparation and/or a therapeutic agent incorporated therein to achieve desirable therapeutic effect(s) such as, e.g., anti-tumor immunity, may vary from subject to subject, depending, for example, on gender, age, and general condition of a subject, type and/or severity of cancer, efficacy of a provided composition, and the like.

In some embodiments, the present disclosure provides technologies such that administration of a composition comprising a biomaterial preparation (e.g., ones described herein) and a modulator of myeloid-derived suppressive cell function (e.g., ones described herein) is sufficient to provide antitumor immunity and thus does not necessarily require administration of, e.g., a tumor antigen, and/or adoptive transfer of immune cells (e.g., T cells) to a subject in need thereof (e.g., as described herein). Accordingly, in some embodiments, technologies provided herein do not include administering a tumor antigen to a subject, e.g., within 1 month or less (including, e.g., within 3 weeks, within 2 weeks, within 1 week, within 5 days, within 3 days, within 1 day, within 12 hours, within 6 hours), after the subject has received a composition as described and/or utilized herein. In certain embodiments, technologies provided herein do not include adoptive transfer of immune cells (e.g., T cells) to a subject, e.g., within 1 month or less (including, e.g., within 3 weeks, within 2 weeks, within 1 week, within 5 days, within 3 days, within 1 day, within 12 hours, within 6 hours) after the subject has received a composition as described and/or utilized herein.

In some embodiments, technologies provided herein are useful for treatment of cancer in a subject. In some embodiments, technologies provided herein are for use in treatment of a resectable tumor. In some embodiments, technologies provided herein are for use in treatment of a solid tumor (e.g., but not limited to a blastoma, a carcinoma, a germ cell tumor, and/or a sarcoma). In some embodiments, technologies provided herein are for use in treatment of lymphoma present in a spleen or a tissue outside of a lymphatic system, e.g., a thyroid or stomach.

In some embodiments, technologies provided herein are useful for treating a cancer including, but not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bile duct cancer; bladder cancer; bone cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma, medulloblastoma); bronchus cancer; carcinoid tumor; cardiac tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ductal carcinoma in situ; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer); hematopoietic cancer (e.g., lymphomas, primary pulmonary lymphomas, bronchus-associated lymphoid tissue lymphomas, splenic lymphomas, nodal marginal zone lymphomas, pediatric B cell non-Hodgkin lymphomas); hemangioblastoma; histiocytosis; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); melanoma; midline tract carcinoma; multiple endocrine neoplasia syndrome; muscle cancer; mesothelioma; nasopharynx cancer; neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); parathyroid cancer; papillary adenocarcinoma; penile cancer (e.g., Paget's disease of the penis and scrotum); pharyngeal cancer; pinealoma; pituitary cancer; pleuropulmonary blastoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; retinoblastoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; stomach cancer; small intestine cancer; sweat gland carcinoma; synovioma; testicular cancer (e.g., seminoma, testicular embryonal carcinoma); thymic cancer; thyroid cancer (e.g., papillary carcinoma of the thyroid, papillary thyroid carcinoma (PTC), medullary thyroid cancer); urethral cancer; uterine cancer; vaginal cancer; vulvar cancer (e.g., Paget's disease of the vulva), or any combination thereof.

In certain embodiments, the cancer is breast cancer. In certain embodiments, the cancer is skin cancer. In certain embodiments, the cancer is melanoma. In certain embodiments, the cancer is lung cancer. In certain embodiments, the cancer is kidney cancer. In certain embodiments, the cancer is liver cancer. In certain embodiments, the cancer is pancreatic cancer. In certain embodiments, the cancer is colorectal cancer. In certain embodiments, the cancer is bladder cancer. In certain embodiments, the cancer is lymphoma. In certain embodiments, the cancer is prostate cancer. In certain embodiments, the cancer is thyroid cancer. In certain embodiments, the cancer is brain cancer. In certain embodiments, the cancer is stomach cancer. In certain embodiments, the cancer is esophageal cancer.

In some embodiments, technologies provided herein are useful in treating adenocarcinoma, adrenal gland cancer, anal cancer, angiosarcoma, appendix cancer, bile duct cancer, bladder cancer, bone cancer, brain cancer, breast cancer, bronchus cancer, carcinoid tumor, cardiac tumor, cervical cancer, choriocarcinoma, chordoma, colorectal cancer, connective tissue cancer, craniopharyngioma, ductal carcinoma in situ, endotheliosarcoma, endometrial cancer, ependymoma, epithelial carcinoma, esophageal cancer, Ewing's sarcoma, eye cancer, familiar hypereosinophilia, gall bladder cancer, gastric cancer, gastrointestinal carcinoid tumor, gastrointestinal stromal tumor (GIST), germ cell cancer, head and neck cancer, hemangioblastoma, histiocytosis, Hodgkin lymphoma, hypopharynx cancer, inflammatory myofibroblastic tumors, intraepithelial neoplasms, immunocytic amyloidosis, Kaposi sarcoma, kidney cancer, liver cancer, lung cancer, leiomyosarcoma (LMS), melanoma, midline tract carcinoma, multiple endocrine neoplasia syndrome, muscle cancer, mesothelioma, myeloproliferative disorder (MPD), nasopharynx cancer, neuroblastoma, neurofibroma, neuroendocrine cancer, non-Hodgkin lymphoma, osteosarcoma, ovarian cancer, pancreatic cancer, paraneoplastic syndromes, parathyroid cancer, papillary adenocarcinoma, penile cancer, pharyngeal cancer, pheochromocytoma, pinealoma, pituitary cancer, pleuropulmonary blastoma, primitive neuroectodermal tumor (PNT), plasma cell neoplasia, prostate cancer, rectal cancer, retinoblastoma, rhabdomyosarcoma, salivary gland cancer, sebaceous gland carcinoma, skin cancer, small bowel cancer, small intestine cancer, soft tissue sarcoma, stomach cancer, sweat gland carcinoma, synovioma, testicular cancer, thymic cancer, thyroid cancer, urethral cancer, uterine cancer, vaginal cancer, vascular cancer, vulvar cancer, or a combination thereof.

In some embodiments, a method provided herein may comprise administering to a target site (e.g., as described herein) in a tumor resection subject a provided composition and, optionally, monitoring the tumor resection site or distal sites for risk or incidence of tumor regrowth or tumor outgrowth in the subject after the administration, e.g., every 3 months or longer after the administration, including, e.g., every 6 months, every 9 months, every year, or longer. When the subject is determined to have risk or incidence of tumor recurrence based on the monitoring report, in some embodiments, a subject can be administered with a second composition (e.g., as described herein) and/or a different treatment regimen (e.g., chemotherapy).

In some embodiments, technologies provided herein may be useful for treating subjects who are suffering from metastatic cancer. For example, in some embodiments, a method provided herein may comprise administering to a target site (e.g., as described herein) in a subject suffering from one or more metastases who has undergone a tumor resection (e.g., surgical resection of a primary tumor) and, optionally, monitoring at least one metastatic site in the subject after the administration, e.g., every 3 months or longer after the administration, including, e.g., every 6 months, every 9 months, every year, or longer. Based on results of the monitoring report, in some embodiments, a subject can be administered with a second composition (e.g., as described herein) and/or a different treatment regimen (e.g., chemotherapy).

In certain embodiments, the methods described herein do not comprise administering a provided composition prior to tumor resection. In certain embodiments, the methods described herein do comprise administering a provided composition prior to tumor resection. In certain embodiments, technologies provided herein comprise administering a provided composition to a tumor resection site concurrently to tumor resection. In certain embodiments, technologies provided herein comprise administering a provided composition to a tumor resection site following tumor resection.

While not necessary, it will be also appreciated that compositions described herein can be administered in combination with one or more additional pharmaceutical agents. For example, compositions can be administered in combination with additional pharmaceutical agents that reduce and/or modify their metabolism, inhibit their excretion, and/or modify their distribution within the body. It will also be appreciated that the additional therapy employed may achieve a desired effect for the same disorder, and/or it may achieve different effects. In certain embodiments, an additional pharmaceutical agent is not adoptively transferred cells. In certain embodiments, an additional pharmaceutical agent is not T cells. In certain embodiments, an additional pharmaceutical agent is administered multiple days or weeks after administration of a composition described herein.

In certain embodiments, a subject being treated is a mammal. In certain embodiments, a subject is a human. In certain embodiments, a subject is a tumor resection human subject. In certain embodiments, a subject is a human patient who has received neoadjuvant (pre-operative) therapy. In certain embodiments, a subject is a human patient who has not received neoadjuvant therapy. In certain embodiments, a subject is a human patient who has received neoadjuvant (pre-operative) chemotherapy. In certain embodiments, a subject is a human patient who has received neoadjuvant radiation therapy. In certain embodiments, a subject is a human patient who has not received neoadjuvant (pre-operative) chemotherapy. In certain embodiments, a subject is a human patient who has received neoadjuvant chemotherapy and/or radiation therapy. In certain embodiments, a subject is a human patient who has not received neoadjuvant radiation therapy. In certain embodiments, a subject is a human patient who has received neoadjuvant molecular targeted therapy. In certain embodiments, a subject is a human patient who has not received neoadjuvant molecular targeted therapy. In certain embodiments, a subject is a human patient who has not received neoadjuvant chemotherapy. In some embodiments, a subject is receiving, has received, or will receive immune checkpoint blockade therapy. In certain embodiments, a subject is receiving immune checkpoint blockade therapy. In some embodiments, a subject is receiving, has received, or will receive certain other cancer therapeutics (e.g., including but not limited to costimulation, oncolytic virus, CAR T cells, transgenic TCRs, TILs, vaccine, BiTE, ADC, cytokines, modulators of innate immunity, or any combination of these). In certain embodiments, a subject is a human patient who has received neoadjuvant immunotherapy, including immune checkpoint blockade (e.g., anti-CTLA-4, anti-PD-1, and/or anti-PD-L1). In certain embodiments, a subject is a human patient who has not received and/or will not receive neoadjuvant immunotherapy, including immune checkpoint blockade (e.g., anti-CTLA-4, anti-PD-1, and/or anti-PD-L1). In certain embodiments, a subject is a human patient whose tumor has not objectively responded to neoadjuvant therapy (as defined by Response Evaluation Criteria in Solid Tumors (RECIST) or immune-related Response Criteria (irRC)) (e.g., stable disease, progressive disease). In certain embodiments, a subject is a human patient whose target lesion has objectively responded and/or is objectively responding to neoadjuvant therapy (e.g., partial response, complete response). Non-target lesions may exhibit an incomplete response, stable disease, or progressive disease. In certain embodiments, a subject is a human patient who would be eligible to receive immunotherapy in an adjuvant (post-operative) setting. In certain embodiments, a subject is a domesticated animal, such as a dog, cat, cow, pig, horse, sheep, or goat. In certain embodiments, a subject is a companion animal such as a dog or cat. In certain embodiments, a subject is a livestock animal such as a cow, pig, horse, sheep, or goat. In certain embodiments, a subject is a zoo animal. In another embodiment, a subject is a research animal, such as a rodent, pig, dog, or non-human primate. In certain embodiments, a subject is a non-human transgenic animal such as a transgenic mouse or transgenic pig.

### V. Kits

The present disclosure also provides kits that find use in practicing technologies as provided herein. In some embodiments, a kit comprises a composition or a pharmaceutical composition described herein and a container (e.g., a vial, ampule, bottle, syringe, and/or dispenser package, or other suitable container). In some embodiments, a kit comprises delivery technologies such as syringes, bags, etc., or components thereof, which may be provided as a single and/or multiple use item. In some embodiments, one or more component(s) of a composition or a pharmaceutical composition described herein are separately provided in one or more containers. For example, individual components of a composition (e.g., ones described herein) may be, in some embodiments, provided in separate containers. In some such embodiments, individual components of a biomaterial (e.g., ones described herein, for example, but not limited to hyaluronic acid, chitosan, poloxamer, etc.) may be each provided independently as dry lyophilized powder, dry particles, or a liquid. In some embodiments, individual components of a composition may be provided as a single mixture in a container. In some such embodiments, a single mixture may be provided as dry lyophilized powder, dry particles, or a liquid (e.g., a homogenous liquid).

In some embodiments, a composition described herein may be provided as a pre-formed polymer network biomaterial (incorporated with a modulator of myeloid-derived suppressive cell function) in a container. In some embodiments, such a pre-formed polymer network biomaterial (e.g., a hydrogel) may be provided in a dried state. In some embodiments, such a pre-formed polymer network biomaterial (in a form of a viscous polymer solution) may be provided in a container.

In some embodiments, provided kits may optionally include a container comprising a pharmaceutical excipient for dilution or suspension of a composition or pharmaceutical composition described herein. In some embodiments, provided kits may include a container comprising an aqueous solution. In some embodiments, provided kits may include a container comprising a buffered solution.

In some embodiments, provided kits may comprise a payload such as a therapeutic agent described herein. For example, in some embodiments, a payload may be provided in a separate container such that it can be added to a biomaterial preparation liquid mixture (e.g., as described herein) prior to administration to a subject. In some embodiments, a payload may be incorporated in a biomaterial preparation described herein.

In certain embodiments, a kit described herein further includes instructions for practicing methods described herein. A kit described herein may also include information as required by a regulatory agency such as the U.S. Food and Drug Administration (FDA). In certain embodiments, information included in kits provided herein is prescribing information, e.g., for treatment for cancer. Instructions may be present in kits in a variety of forms, one or more of which may be present in the kits. One form in which these instructions may be present is as printed information on a suitable medium or substrate, e.g., a piece or pieces of paper on which the information is printed, in the packaging of kits, in a package insert, etc. Yet another means may be a computer readable medium, e.g., diskette, CD, USB drive, etc., on which instructional information has been recorded. Yet another means that may be present is a website address which may be used via the internet to access instructional information. Any convenient means may be present in the kits.

Other features of the invention will become apparent in the course of the following description of exemplary embodiments, which are given for illustration of the invention and are not intended to be limiting thereof.

### Exemplification

In order that the invention described herein may be more fully understood, the following examples are set forth. It should be understood that these examples are for illustrative purposes only and are not to be construed as limiting this invention in any manner.

### Example 1. Characterization of Exemplary Compositions for Incorporation and Release of Myeloid-Derived Suppressive Cell Modulators

In some embodiments, exemplary compositions can be useful to provide release of one or more payloads (e.g., myeloid-derived suppressive cell modulators) incorporated therein over a period of time. The present Example describes characterization of certain test compositions comprising biomaterial compositions as described herein (e.g., which may comprise a poloxamer and/or a carbohydrate polymer e.g., hyaluronic acid and/or chitosan or a variant thereof) with respect to release of a modulator of myeloid-derived suppressive cell function incorporated therein over a period of time. In some embodiments, an incorporated modulator of myeloid-derived suppressive cell function may be or comprise a hydrophilic agent. In some such embodiments, at least 10% (including, e.g., at least 10%, at least 20%, at least 30%, at least 40%, at least 50%, at least 60%, at least 70%, at least 80%, or more) of an incorporated hydrophilic modulator of myeloid-derived suppressive cell function may be released over a period of 6 hours, 12 hours, 18 hours, 24 hours, 48 hours, 72 hours, or longer. In some embodiments, an incorporated modulator of myeloid-derived suppressive cell function may be or comprise a lipophilic agent. In some such embodiments, at least 10% (including, e.g., at least 10%, at least 20%, at least 30%, at least 40%, at least 50%, at least 60%, at least 70%, at least 80%, or more) of an incorporated lipophilic modulator of myeloid-derived suppressive cell function may be released over a period of 6 hours, 12 hours, 18 hours, 24 hours, 48 hours, 72 hours, or longer.

In some embodiments, the release kinetics of an incorporated modulator of myeloid-derived suppressive cell function from exemplary compositions can be assessed in-vitro. In certain embodiments, in-vitro release rates of exemplary modulator of myeloid-derived suppressive cell function can be assessed at 37° C. in PBS (pH 7.4). In some embodiments, at least 10%, at least 20%, at least 30%, at least 40%, at least 50%, at least 60%, at least 70%, at least 80%, or at least 90% of an exemplary modulator of myeloid-derived suppressive cell function is released within about 12 hours from the composition preparation test starting time point. In some embodiments, less than 100%, less than 90%, less than 80%, less than 70%, less than 60%, less than 50%, less than 40%, less than 30%, less than 20%, or less than 10% an exemplary modulator of myeloid-derived suppressive cell function is released within about 3 hours from the composition preparation test starting time point.

In some embodiments, the release kinetics of an incorporated modulator of myeloid-derived suppressive cell function from exemplary compositions can be assessed in-vivo. In certain embodiments, in-vivo release rates of exemplary modulators of myeloid-derived suppressive cell function can be assessed by administering a composition to a mammary fat pad of a mouse subject. In some embodiments, at least 10%, at least 20%, at least 30%, at least 40%, at least 50%, at least 60%, at least 70%, at least 80%, or at least 90% of an exemplary modulator of myeloid-derived suppressive cell function is released in-vivo within about 12 hours from the composition implantation time point. In some embodiments, less than 100%, less than 90%, less than 80%, less than 70%, less than 60%, less than 50%, less than 40%, less than 30%, less than 20%, or less than 10% of an exemplary compositions volume and/or weight is present within about 4 month from the composition implantation time point.

### Example 2. Preparation and Uses of Exemplary Composition Described Herein

The present Example describes identification and/or characterization of an exemplary composition comprising a polymeric biomaterial and a modulator of myeloid-derived suppressive cell function, in particular by assessing its ability to extend survival of one or more subjects who have undergone a tumor resection. Accordingly, the present Example also describes identification and/or characterization of an exemplary composition comprising a polymeric biomaterial and a modulator of myeloid-derived suppressive cells that may be useful for cancer treatment (e.g., as described herein). In some embodiments, such an exemplary composition comprising a polymeric biomaterial and a modulator of myeloid-derived suppressive cells may inhibit, modulate, and/or deplete myeloid-derived suppressive cells (e.g., neutrophils).

In some embodiments, administration of a composition comprising a polymeric biomaterial and a modulator of myeloid-derived suppressive cells to a target site following a tumor resection increases survival of a subject who has undergone a tumor resection, as compared to that observed when such a composition is not administered (e.g., a polymeric biomaterial without a modulator of myeloid-derived suppressive cells).

In some embodiments, an animal model of cancer can be used to identify and/or characterize composition comprising a polymeric biomaterial and a modulator of myeloid-derived suppressive cells. For example, a tumor resection is performed on a tumor-bearing mouse, and a composition described herein, e.g., composition comprising a polymeric biomaterial and a modulator of myeloid-derived suppressive cells is administered to the tumor resection site. The survival of treated subjects is then monitored. In some embodiments, a composition comprising a polymeric biomaterial and a modulator of myeloid-derived suppressive cells is considered and/or determined to be useful in accordance with the present disclosure when it is characterized, in that when tested in vivo as described in the present Example, it extends survival of a treated subject, e.g., by at least 1 week, at least 2 weeks, at least 3 weeks, at least 4 weeks, at least 2 months, at least 3 months, at least 4 months, at least 5 months, at least 6 months, or longer, as compared to that observed in a control reference (e.g., a control in which a composition comprising a polymeric biomaterial and a modulator of myeloid-derived suppressive cells is not administered. For example, in some embodiments, a control reference may be administration of a polymeric biomaterial in the absence of a modulator of myeloid-derived suppressive cell function. In some embodiments, a control reference may be administration of a modulator of myeloid-derived suppressive cell function in solution. Alternatively, in some embodiments, a provided composition comprising a biomaterial preparation and a modulator of myeloid-derived suppressive cells is considered and/or determined to be useful for treatment of cancer (including, e.g., prevention or reduction in the likelihood of tumor relapse or metastasis) in accordance with the present disclosure when such a composition, after administration at a tumor resection site, reduces incidence of tumor recurrence and/or metastasis after the tumor resection (e.g., at least 1 month after tumor resection when the test subject is a mouse subject, or at least 3 months after tumor resection when the test subject is a human subject), for example, by at least 10% or more (comprising, e.g., at least 20%, at least 30%, at least 40%, at least 50%, at least 60%, at least 70%, at least 80%, at least 90%, or more) as compared to that observed in a control reference (e.g., as described above).

In some embodiments, female BALB/cJ mice are inoculated orthotopically with 100,000 breast cancer cells (e.g., 4T1-Luc2 cells). Ten days later, tumors are surgically resected, and either (i) a composition described herein, e.g., a composition comprising a polymeric biomaterial and a modulator of myeloid-derived suppressive cells, (ii) a composition comprising a polymeric biomaterial without a modulator of myeloid-derived suppressive cells, and/or (iii) a negative control composition (e.g., a buffered solution without such a composition) is administered into the resection cavity. Animal survival can be monitored to inspect for induction of antitumor immunity. In some embodiments, to confirm that a composition comprising a polymeric biomaterial and a modulator of myeloid-derived suppressive cells functions mechanistically, for example, by modulating recruitment and/or survival and/or proliferation of myeloid-derived suppressive cells (e.g., neutrophils), animal survival may be monitored following inhibition of recruitment and/or survival and/or proliferation of myeloid-derived suppressive cells (e.g., neutrophils). In some embodiments, to confirm that an administered composition comprising a polymeric biomaterial and a modulator of myeloid-derived suppressive cells functions mechanistically by modulating myeloid-derived suppressive cell effector function, animal survival may be monitored following modulating myeloid-derived suppressive cell effector function (e.g., as described herein).

To assess whether an administered composition induces an adaptive antitumor immune response, animal survival may be monitored following depletion of particular leukocyte subsets (e.g., NK cells, CD4⁺ T cells, or CD8⁺ T cells).

Exemplary liquidpreparations: In some embodiments, a liquid preparation of a composition comprising a polymeric biomaterial and a modulator of myeloid-derived suppressive cells is prepared as follows. For example, in one instance, a 1-5 weight percent (wt %) chitosan (e.g., but not limited to carboxymethyl chitosan) and Poloxamer 407 (P407) at a concentration of 12.5% or lower (e.g., in some embodiments 6-11%) is prepared in a buffered system that is appropriate for injection administration. In some embodiments, a 2.5 weight percent (wt %) carboxymethyl chitosan (CMCH) (e.g., obtained from Heppe Medical Chitosan, Part Number 43002, Lot Number 312-210519-02) and Poloxamer 407 (P407) at a concentration of 12.5% or lower (e.g., in some embodiments 6-11 wt %) is prepared in a buffered system that is appropriate for injection administration. In another instance, a 5 wt % CMCH (e.g., obtained from Heppe Medical Chitosan, Part Number 43002, Lot Number 312-210519-02) and P407 at a concentration of 12.5% or lower (e.g., in some embodiments 6-11 wt %) is prepared in a buffered system that is appropriate for injection administration. In another instance, a 1-10 wt % low molecular weight (<500 kDa (e.g., in some embodiments 100-200 kDa)) hyaluronic acid (HA) and P407 at a concentration of 6-11 wt % (e.g., in some embodiments 10 wt %) is prepared in a buffered system that is appropriate for injection administration. In another instance, a 1-5 wt % high molecular weight (>500 kDa (e.g., in some embodiments 700-800 kDa)) hyaluronic acid (HA) and P407 at a concentration of 6-11 wt % (e.g., in some embodiments 9 wt %) is prepared in a buffered system that is appropriate for injection administration. In another instance, a 1-5 wt % high molecular weight (>500 kDa (e.g., in some embodiments 700-800 kDa)) hyaluronic acid (HA) and P407 at a concentration of 6-11 wt % (e.g., in some embodiments 11 wt %) is prepared in a buffered system that is appropriate for injection administration. For example, in some embodiments, such a buffered system has a physiological pH. The liquid preparation is loaded into a 1 mL syringe for administration. Modulator(s) of myeloid-derived suppressive cells are mixed with the polymeric biomaterial compositions.

Exemplary mouse tumor models: In some embodiments, animal experiments are performed using 6-8 weeks old female BALB/c mice (Jackson Laboratories, #000651). For animal survival studies, approximately 10⁵ 4T1-Luc2 cells are inoculated orthotopically into the fourth mammary fat pad of a mouse. Tumor sizes are measured with calipers. Following size-matching, mice are randomly assigned to treatment groups, and surgery is performed on day 10 after tumor inoculation. For primary tumor resection, mice are anesthetized with 2% isoflurane, the tumor is resected, and a composition comprising a polymeric biomaterial and a modulator of myeloid-derived suppressive cells is administered to a tumor resection site at the time of surgery. In certain embodiments, a composition comprising a polymeric biomaterial and a modulator of myeloid-derived suppressive cells gels at body temperature and is administered to a tumor resection site at the time of surgery.

### Example 3. Preparation and Uses of Exemplary Composition Described Herein Comprising a BTK Inhibitor

In certain embodiments, a group of tumor resection mice (e.g., prepared as described in Example 2) receiving a composition as described herein comprising a polymeric biomaterial and a BTK inhibitor (e.g., zanubrutinib) at a tumor resection site survive over a longer period of time (e.g., by at least 10%, 20%, 30%, 40%, 50%, or more), as compared to the control tumor resection mice receiving a control reference composition without a BTK inhibitor. In addition, the group of tumor resection mice receiving said composition comprising a polymeric biomaterial and a BTK inhibitor (e.g., zanubrutinib) exhibit a higher survival rate as compared to the control tumor resection mice receiving a control reference composition without a BTK inhibitor.

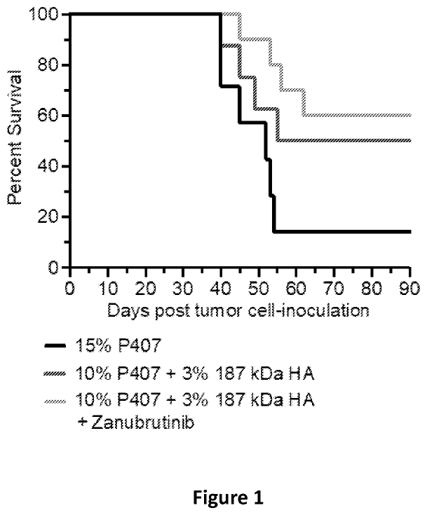

As shown in FIG. 1, the group of tumor resection mice receiving a composition comprising a polymeric biomaterial of 10% w/w poloxamer 407 and 3% w/w 187 kDa HA with a BTK inhibitor (e.g., zanubrutinib, for example, in some embodiments at a dose of 1.25 mg/mouse) at a tumor resection site survived over a longer period of time as compared to the control group receiving a composition comprising 10% w/w poloxamer 407 and 3% w/w 187 kDa HA without a BTK inhibitor, and to the control group receiving a composition comprising 15% w/w poloxamer 407.

### Example 4. Preparation and Uses of Exemplary Composition Described Herein Comprising a CSF1R Inhibitor

In certain embodiments, a group of tumor resection mice (e.g., prepared as described in Example 2) receiving a composition as described herein comprising a polymeric biomaterial and a CSF1R inhibitor (e.g., edicotinib) at a tumor resection site survive over a longer period of time (e.g., by at least 10%, 20%, 30%, 40%, 50%, or more), as compared to the control tumor resection mice receiving a control reference composition without a CSF1R inhibitor. In addition, the group of tumor resection mice receiving said composition comprising a polymeric biomaterial and a CSF1R inhibitor (e.g., edicotinib) exhibit a higher survival rate as compared to the control tumor resection mice receiving a control reference composition without a CSF1R inhibitor.

### Example 5. Preparation and Uses of Exemplary Composition Described Herein Comprising a Tyrosine Kinase Inhibitor

In certain embodiments, a group of tumor resection mice (e.g., prepared as described in Example 2) receiving a composition as described herein comprising a polymeric biomaterial and a tyrosine kinase inhibitor (e.g., dasatinib) at a tumor resection site survive over a longer period of time (e.g., by at least 10%, 20%, 30%, 40%, 50%, or more), as compared to the control tumor resection mice receiving a control reference composition without a tyrosine kinase inhibitor. In addition, the group of tumor resection mice receiving said composition comprising a polymeric biomaterial and a tyrosine kinase inhibitor (e.g., dasatinib) exhibit a higher survival rate as compared to the control tumor resection mice receiving a control reference composition without a tyrosine kinase inhibitor.

### Example 6. Preparation and Uses of Exemplary Composition Described Herein Comprising a COX1 and/or COX2 Inhibitor

In certain embodiments, a group of tumor resection mice (e.g., prepared as described in Example 2) receiving a composition as described herein comprising a polymeric biomaterial and a COX1 and/or COX2 inhibitor (e.g., ketorolac) at a tumor resection site survive over a longer period of time (e.g., by at least 10%, 20%, 30%, 40%, 50%, or more), as compared to the control tumor resection mice receiving a control reference composition without a COX1 and/or COX2 inhibitor. In addition, the group of tumor resection mice receiving said composition comprising a polymeric biomaterial and a COX1 and/or COX2 inhibitor (e.g., ketorolac) exhibit a higher survival rate as compared to the control tumor resection mice receiving a control reference composition without a COX1 and/or COX2 inhibitor.

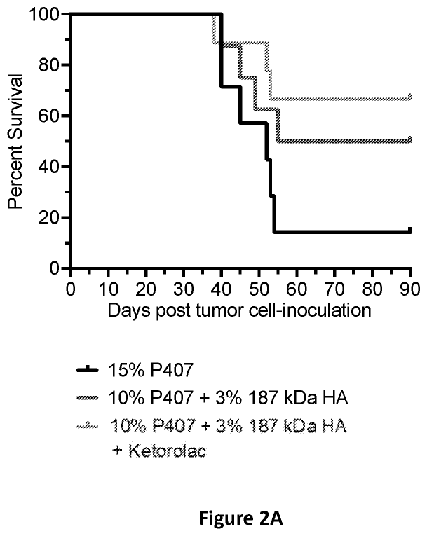

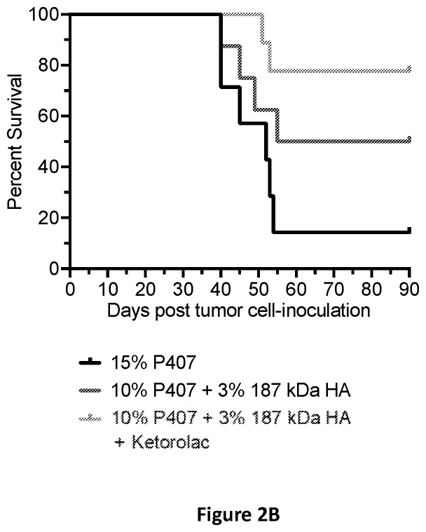

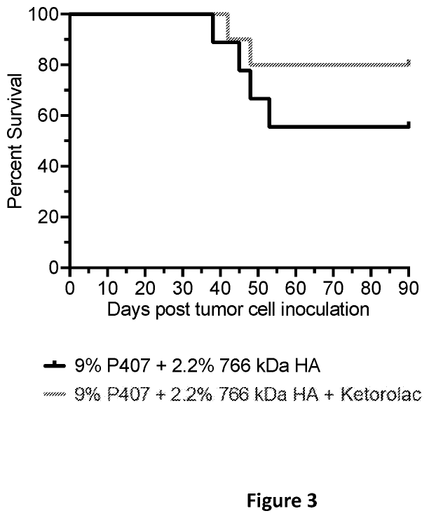

As shown in FIGS. 2A and 2B, the groups of tumor resection mice receiving a composition comprising a polymeric biomaterial of 10% w/w poloxamer 407 and 3% w/w 187 kDa HA with a COX1 and/or COX2 inhibitor (e.g., Ketorolac, for example in some embodiments at a dose of 6 mg/mouse or 9 mg/mouse) at a tumor resection site survived over a longer period of time as compared to the control groups receiving a composition of 10% w/w poloxamer 407 and 3% w/w 187 kDa HA without a COX1 and/or COX2 inhibitor, and to the control groups receiving a composition comprising 15% w/w poloxamer 407. As shown in FIG. 3, the group of tumor resection mice receiving a composition of a polymeric biomaterial of 9% w/w poloxamer 407 and 2.2% w/w 766 kDa HA with a COX1 and/or COX2 inhibitor (e.g., Ketorolac including, e.g., a salt of ketorolac such as, e.g., but not limited to ketorolac tromethamine, for example, in some embodiments at a dose of 1.2 mg/mouse) at a tumor resection site survived over a longer period of time as compared to the control group receiving a composition comprising 9% w/w poloxamer 407 and 2.2% w/w 766 kDa HA without a COX1 and/or COX2 inhibitor.

In certain embodiments, a group of tumor resection mice (e.g., prepared as described in Example 2) may receive a composition as described herein comprising a polymeric biomaterial (e.g., comprising a poloxamer) and a COX1 and/or COX2 inhibitor (e.g., lornoxicam) at a tumor resection site. In some embodiments, such a group of tumor resection mice may survive over a longer period of time (e.g., by at least 10%, 20%, 30%, 40%, 50%, or more), as compared to the control tumor resection mice receiving a control reference composition without a COX1 and/or COX2 inhibitor. In addition, the group of tumor resection mice receiving said composition comprising a polymeric biomaterial and a COX1 and/or COX2 inhibitor (e.g., lornoxicam) may exhibit a higher survival rate as compared to the control tumor resection mice receiving a control reference composition without a COX1 and/or COX2 inhibitor.

### Example 7. Preparation and Uses of Exemplary Composition Described Herein Comprising a Specialized Pro-Resolving Mediator

In certain embodiments, a group of tumor resection mice (e.g., prepared as described in Example 2) may receive a composition as described herein comprising a polymeric biomaterial (e.g., comprising a poloxamer) and a specialized pro-resolving mediator (e.g., RvD2) at a tumor resection site. In some embodiments, such a group of tumor resection mice may survive over a longer period of time (e.g., by at least 10%, 20%, 30%, 40%, 50%, or more), as compared to the control tumor resection mice receiving a control reference composition without a specialized pro-resolving mediator. In addition, the group of tumor resection mice receiving said composition comprising a polymeric biomaterial and a specialized pro-resolving mediator (e.g., RvD2) may exhibit a higher survival rate as compared to the control tumor resection mice receiving a control reference composition without a specialized pro-resolving mediator.

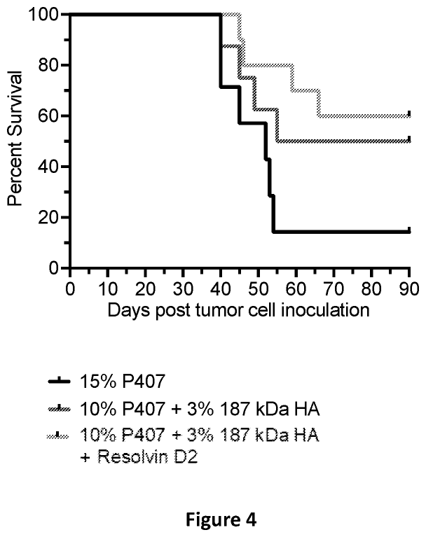

As shown in FIG. 4, the group of tumor resection mice receiving a composition comprising a polymeric biomaterial of 10% w/w poloxamer 407 and 3% w/w 187 kDa HA with a specialized pro-resolving mediator (e.g., Resolvin D2 (RvD2), for example, in some embodiments at a dose of 2.5 μg/mouse) at a tumor resection site survived over a longer period of time as compared to the control group receiving a composition of 10% w/w poloxamer 407 and 3% w/w 187 kDa HA without a specialized pro-resolving mediator, and to the control group receiving a composition comprising 15% w/w poloxamer 407.

In certain embodiments, a group of tumor resection mice (e.g., prepared as described in Example 2) may receive a composition as described herein comprising a polymeric biomaterial (e.g., comprising a poloxamer) and a specialized pro-resolving mediator (e.g., LXA4) at a tumor resection site. In some embodiments, such a group of tumor resection mice may survive over a longer period of time (e.g., by at least 10%, 20%, 30%, 40%, 50%, or more), as compared to the control tumor resection mice receiving a control reference composition without a specialized pro-resolving mediator. In addition, the group of tumor resection mice receiving said composition comprising a polymeric biomaterial and a specialized pro-resolving mediator (e.g., LXA4) may exhibit a higher survival rate as compared to the control tumor resection mice receiving a control reference composition without a specialized pro-resolving mediator.

### Example 8. Preparation and Uses of Exemplary Composition Described Herein Comprising a PDE5 Inhibitor

In certain embodiments, a group of tumor resection mice (e.g., prepared as described in Example 2) receiving a composition as described herein comprising a polymeric biomaterial and a PDE5 inhibitor (e.g., sildenafil) at a tumor resection site survive over a longer period of time (e.g., by at least 10%, 20%, 30%, 40%, 50%, or more), as compared to the control tumor resection mice receiving a control reference composition without a PDE5 inhibitor. In addition, the group of tumor resection mice receiving said composition comprising a polymeric biomaterial and a PDE5 inhibitor (e.g., sildenafil) exhibit a higher survival rate as compared to the control tumor resection mice receiving a control reference composition without a PDE5 inhibitor.

### Example 9. Preparation and Uses of Exemplary Composition Described Herein Comprising a IAP Inhibitor

In certain embodiments, a group of tumor resection mice (e.g., prepared as described in Example 2) receiving a composition as described herein comprising a polymeric biomaterial and an IAP inhibitor (e.g., birinapant) at a tumor resection site survive over a longer period of time (e.g., by at least 10%, 20%, 30%, 40%, 50%, or more), as compared to the control tumor resection mice receiving a control reference composition without an IAP inhibitor. In addition, the group of tumor resection mice receiving said composition comprising a polymeric biomaterial and an IAP inhibitor (e.g., birinapant) exhibit a higher survival rate as compared to the control tumor resection mice receiving a control reference composition without an IAP inhibitor.

### Example 10. Preparation and Uses of Exemplary Composition Described Herein Comprising a TGFβR1 Inhibitor

In certain embodiments, a group of tumor resection mice (e.g., prepared as described in Example 2) receiving a composition as described herein comprising a polymeric biomaterial and a TGFβR1 inhibitor (e.g., galunisertib) at a tumor resection site survive over a longer period of time (e.g., by at least 10%, 20%, 30%, 40%, 50%, or more), as compared to the control tumor resection mice receiving a control reference composition without a TGFβR1 inhibitor. In addition, the group of tumor resection mice receiving said composition comprising a polymeric biomaterial and a TGFβR1 inhibitor (e.g., galunisertib) exhibit a higher survival rate as compared to the control tumor resection mice receiving a control reference composition without a TGFβR1 inhibitor.

### Example 11. Preparation and Uses of Exemplary Composition Described Herein Comprising an Inhibitor of a C-C Motif Chemokine Signaling Pathway and/or a C-X-C Motif Chemokine Signaling Pathway

In certain embodiments, a group of tumor resection mice (e.g., prepared as described in Example 2) receiving a composition as described herein comprising a polymeric biomaterial and a CCR2 inhibitor (e.g., BMS-813160) at a tumor resection site survive over a longer period of time (e.g., by at least 10%, 20%, 30%, 40%, 50%, or more), as compared to the control tumor resection mice receiving a control reference composition without a CCR2 inhibitor. In addition, the group of tumor resection mice receiving said composition comprising a polymeric biomaterial and a CCR2 inhibitor (e.g., BMS-813160) exhibit a higher survival rate as compared to the control tumor resection mice receiving a control reference composition without a CCR2 inhibitor.

In certain embodiments, a group of tumor resection mice (e.g., prepared as described in Example 2) receiving a composition as described herein comprising a polymeric biomaterial and a CCR2 inhibitor (e.g., BMS CCR2 22) at a tumor resection site survive over a longer period of time (e.g., by at least 10%, 20%, 30%, 40%, 50%, or more), as compared to the control tumor resection mice receiving a control reference composition without a CCR2 inhibitor. In addition, the group of tumor resection mice receiving said composition comprising a polymeric biomaterial and a CCR2 inhibitor (e.g., BMS CCR2 22) exhibit a higher survival rate as compared to the control tumor resection mice receiving a control reference composition without a CCR2 inhibitor.

In certain embodiments, a group of tumor resection mice (e.g., prepared as described in Example 2) receiving a composition as described herein comprising a polymeric biomaterial and a CCR2 inhibitor (e.g., MK-0812) at a tumor resection site survive over a longer period of time (e.g., by at least 10%, 20%, 30%, 40%, 50%, or more), as compared to the control tumor resection mice receiving a control reference composition without a CCR2 inhibitor. In addition, the group of tumor resection mice receiving said composition comprising a polymeric biomaterial and a CCR2 inhibitor (e.g., MK-0812) exhibit a higher survival rate as compared to the control tumor resection mice receiving a control reference composition without a CCR2 inhibitor.

In certain embodiments, a group of tumor resection mice (e.g., prepared as described in Example 2) receiving a composition as described herein comprising a polymeric biomaterial and a CCR2 inhibitor (e.g., CCX872) at a tumor resection site survive over a longer period of time (e.g., by at least 10%, 20%, 30%, 40%, 50%, or more), as compared to the control tumor resection mice receiving a control reference composition without a CCR2 inhibitor. In addition, the group of tumor resection mice receiving said composition comprising a polymeric biomaterial and a CCR2 inhibitor (e.g., CCX872) exhibit a higher survival rate as compared to the control tumor resection mice receiving a control reference composition without a CCR2 inhibitor.

In certain embodiments, a group of tumor resection mice (e.g., prepared as described in Example 2) receiving a composition as described herein comprising a polymeric biomaterial and a CCR2 inhibitor (e.g., PF-04136309) at a tumor resection site survive over a longer period of time (e.g., by at least 10%, 20%, 30%, 40%, 50%, or more), as compared to the control tumor resection mice receiving a control reference composition without a CCR2 inhibitor. In addition, the group of tumor resection mice receiving said composition comprising a polymeric biomaterial and a CCR2 inhibitor (e.g., PF-04136309) exhibit a higher survival rate as compared to the control tumor resection mice receiving a control reference composition without a CCR2 inhibitor.

In certain embodiments, a group of tumor resection mice (e.g., prepared as described in Example 2) receiving a composition as described herein comprising a polymeric biomaterial and a CCL2 inhibitor (e.g., bindarit) at a tumor resection site survive over a longer period of time (e.g., by at least 10%, 20%, 30%, 40%, 50%, or more), as compared to the control tumor resection mice receiving a control reference composition without a CCL2 inhibitor. In addition, the group of tumor resection mice receiving said composition comprising a polymeric biomaterial and a CCL2 inhibitor (e.g., bindarit) exhibit a higher survival rate as compared to the control tumor resection mice receiving a control reference composition without a CCL2 inhibitor.

In certain embodiments, a group of tumor resection mice (e.g., prepared as described in Example 2) receiving a composition as described herein comprising a polymeric biomaterial and a CXCR1/2 inhibitor (e.g., reparixin) at a tumor resection site survive over a longer period of time (e.g., by at least 10%, 20%, 30%, 40%, 50%, or more), as compared to the control tumor resection mice receiving a control reference composition without a CXCR1/2 inhibitor. In addition, the group of tumor resection mice receiving said composition comprising a polymeric biomaterial and a CXCR1/2 inhibitor (e.g., reparixin) exhibit a higher survival rate as compared to the control tumor resection mice receiving a control reference composition without a CXCR1/2 inhibitor.

In certain embodiments, a group of tumor resection mice (e.g., prepared as described in Example 2) receiving a composition as described herein comprising a polymeric biomaterial and a CXCR4/CXCL12 signaling inhibitor (e.g., plerixafor) at a tumor resection site survive over a longer period of time (e.g., by at least 10%, 20%, 30%, 40%, 50%, or more), as compared to the control tumor resection mice receiving a control reference composition without a CXCR4/CXCL12 signaling inhibitor. In addition, the group of tumor resection mice receiving said composition comprising a polymeric biomaterial and a CXCR4/CXCL12 signaling inhibitor (e.g., plerixafor) exhibit a higher survival rate as compared to the control tumor resection mice receiving a control reference composition without a CXCR4/CXCL12 signaling inhibitor.

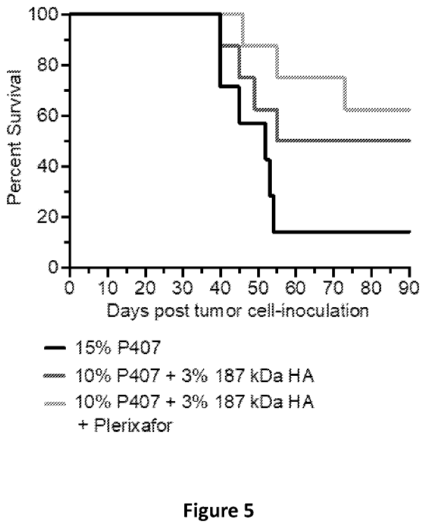

As shown in FIG. 5, the group of tumor resection mice receiving a composition of a polymeric biomaterial comprising 10% w/w poloxamer 407 and 3% w/w 187 kDa HA with a CXCR4/CXCL12 signaling inhibitor (e.g., Plerixafor, for example in some embodiments at a dose of 1.25 mg/mouse) at a tumor resection site survived over a longer period of time as compared to the control group receiving a composition of 10% w/w poloxamer 407 and 3% w/w 187 kDa HA without a CXCR4/CXCL12 signaling inhibitor, and to the control group receiving a composition of 15% w/w poloxamer 407.

### Example 12. Preparation and Uses of Exemplary Composition Described Herein Comprising Metformin

In certain embodiments, a group of tumor resection mice (e.g., prepared as described in Example 2) receiving a composition as described herein comprising a polymeric biomaterial and metformin at a tumor resection site survive over a longer period of time (e.g., by at least 10%, 20%, 30%, 40%, 50%, or more), as compared to the control tumor resection mice receiving a control reference composition without metformin. In addition, the group of tumor resection mice receiving said composition comprising a polymeric biomaterial and metformin exhibit a higher survival rate as compared to the control tumor resection mice receiving a control reference composition without metformin.

### Example 13. Preparation and Uses of Exemplary Composition Described Herein Comprising a NOD1/2 Inhibitor

In certain embodiments, a group of tumor resection mice (e.g., prepared as described in Example 2) receiving a composition as described herein comprising a polymeric biomaterial and a NOD1/2 inhibitor (e.g., M-TriDAP) at a tumor resection site survive over a longer period of time (e.g., by at least 10%, 20%, 30%, 40%, 50%, or more), as compared to the control tumor resection mice receiving a control reference composition without a NOD1/2 inhibitor. In addition, the group of tumor resection mice receiving said composition comprising a polymeric biomaterial and a NOD1/2 inhibitor (e.g., M-TriDAP) exhibit a higher survival rate as compared to the control tumor resection mice receiving a control reference composition without a NOD1/2 inhibitor.

### Example 14. Preparation and Uses of Exemplary Composition Described Herein Comprising a TREM-1 and/or TREM-2 Inhibitor

In certain embodiments, a group of tumor resection mice (e.g., prepared as described in Example 2) receiving a composition as described herein comprising a polymeric biomaterial and a TREM-1 inhibitor (e.g., PY159) at a tumor resection site survive over a longer period of time (e.g., by at least 10%, 20%, 30%, 40%, 50%, or more), as compared to the control tumor resection mice receiving a control reference composition without a TREM-1 inhibitor. In addition, the group of tumor resection mice receiving said composition comprising a polymeric biomaterial and a TREM-1 inhibitor (e.g., PY159) exhibit a higher survival rate as compared to the control tumor resection mice receiving a control reference composition without a TREM-1 inhibitor.

In certain embodiments, a group of tumor resection mice (e.g., prepared as described in Example 2) receiving a composition as described herein comprising a polymeric biomaterial and a TREM-2 inhibitor (e.g., PY314) at a tumor resection site survive over a longer period of time (e.g., by at least 10%, 20%, 30%, 40%, 50%, or more), as compared to the control tumor resection mice receiving a control reference composition without a TREM-2 inhibitor. In addition, the group of tumor resection mice receiving said composition comprising a polymeric biomaterial and a TREM-2 inhibitor (e.g., PY314) exhibit a higher survival rate as compared to the control tumor resection mice receiving a control reference composition without a TREM-2 inhibitor.

### Example 15. Preparation and Uses of Exemplary Composition Described Herein Comprising a Cathepsin G Inhibitor

In certain embodiments, a group of tumor resection mice (e.g., prepared as described in Example 2) receiving a composition as described herein comprising a polymeric biomaterial and a cathepsin G inhibitor (e.g., aprotinin) at a tumor resection site survive over a longer period of time (e.g., by at least 10%, 20%, 30%, 40%, 50%, or more), as compared to the control tumor resection mice receiving a control reference composition without a cathepsin G inhibitor. In addition, the group of tumor resection mice receiving said composition comprising a polymeric biomaterial and a cathepsin G inhibitor (e.g., aprotinin) exhibit a higher survival rate as compared to the control tumor resection mice receiving a control reference composition without a cathepsin G inhibitor.

### Example 16. Preparation and Uses of Exemplary Composition Described Herein Comprising a Elastase Inhibitor

In certain embodiments, a group of tumor resection mice (e.g., prepared as described in Example 2) receiving a composition as described herein comprising a polymeric biomaterial and an elastase inhibitor (e.g., BMS-P5) at a tumor resection site survive over a longer period of time (e.g., by at least 10%, 20%, 30%, 40%, 50%, or more), as compared to the control tumor resection mice receiving a control reference composition without an elastase inhibitor. In addition, the group of tumor resection mice receiving said composition comprising a polymeric biomaterial and an elastase inhibitor (e.g., BMS-P5) exhibit a higher survival rate as compared to the control tumor resection mice receiving a control reference composition without an elastase inhibitor.

In certain embodiments, a group of tumor resection mice (e.g., prepared as described in Example 2) receiving a composition as described herein comprising a polymeric biomaterial and an elastase inhibitor (e.g., GSK199) at a tumor resection site survive over a longer period of time (e.g., by at least 10%, 20%, 30%, 40%, 50%, or more), as compared to the control tumor resection mice receiving a control reference composition without an elastase inhibitor. In addition, the group of tumor resection mice receiving said composition comprising a polymeric biomaterial and an elastase inhibitor (e.g., GSK199) exhibit a higher survival rate as compared to the control tumor resection mice receiving a control reference composition without an elastase inhibitor.

In certain embodiments, a group of tumor resection mice (e.g., prepared as described in Example 2) receiving a composition as described herein comprising a polymeric biomaterial and an elastase inhibitor (e.g., GSK484) at a tumor resection site survive over a longer period of time (e.g., by at least 10%, 20%, 30%, 40%, 50%, or more), as compared to the control tumor resection mice receiving a control reference composition without an elastase inhibitor. In addition, the group of tumor resection mice receiving said composition comprising a polymeric biomaterial and an elastase inhibitor (e.g., GSK484) exhibit a higher survival rate as compared to the control tumor resection mice receiving a control reference composition without an elastase inhibitor.

### Example 17. Preparation and Uses of Exemplary Composition Described Herein Comprising a CD47 Inhibitor

In certain embodiments, a group of tumor resection mice (e.g., prepared as described in Example 2) receiving a composition as described herein comprising a polymeric biomaterial and a CD47 inhibitor (e.g., magrolimab) at a tumor resection site survive over a longer period of time (e.g., by at least 10%, 20%, 30%, 40%, 50%, or more), as compared to the control tumor resection mice receiving a control reference composition without a CD47 inhibitor. In addition, the group of tumor resection mice receiving said composition comprising a polymeric biomaterial and a CD47 inhibitor (e.g., magrolimab) exhibit a higher survival rate as compared to the control tumor resection mice receiving a control reference composition without a CD47 inhibitor.

### Example 18. Preparation and Uses of Exemplary Composition Described Herein Comprising a MMP Inhibitor

In certain embodiments, a group of tumor resection mice (e.g., prepared as described in Example 2) receiving a composition as described herein comprising a polymeric biomaterial and a MMP inhibitor (e.g., JNJ0966) at a tumor resection site survive over a longer period of time (e.g., by at least 10%, 20%, 30%, 40%, 50%, or more), as compared to the control tumor resection mice receiving a control reference composition without a MMP inhibitor. In addition, the group of tumor resection mice receiving said composition comprising a polymeric biomaterial and a MMP inhibitor (e.g., JNJ0966) exhibit a higher survival rate as compared to the control tumor resection mice receiving a control reference composition without a MMP inhibitor.

### Example 19. Preparation and Uses of Exemplary Composition Described Herein Comprising an Adenosine Pathway Inhibitor

In certain embodiments, a group of tumor resection mice (e.g., prepared as described in Example 2) receiving a composition as described herein comprising a polymeric biomaterial and an A2A and/or A2B adenosine receptor inhibitor (e.g., AB928, aka etrumadenant) at a tumor resection site survive over a longer period of time (e.g., by at least 10%, 20%, 30%, 40%, 50%, or more), as compared to the control tumor resection mice receiving a control reference composition without an A2A and/or A2B receptor inhibitor. In addition, the group of tumor resection mice receiving said composition comprising a polymeric biomaterial and an A2A and/or A2B adenosine receptor inhibitor (e.g., AB928) exhibit a higher survival rate as compared to the control tumor resection mice receiving a control reference composition without an A2A and/or A2B receptor inhibitor.

As shown in FIG. 6, the group of tumor resection mice receiving a composition comprising a polymeric biomaterial that comprises 10% w/w poloxamer 407 and 3% w/w 187 kDa HA with an A2A and/or A2B adenosine receptor inhibitor (e.g., AB928, for example in some embodiments at a dose of 1.25 mg/mouse) at a tumor resection site survived over a longer period of time as compared to the control group receiving a composition comprising 10% w/w poloxamer 407 and 3% w/w 187 kDa HA without an A2A and/or A2B adenosine receptor inhibitor, and to the control group receiving a composition comprising 15% w/w poloxamer 407.

In certain embodiments, a group of tumor resection mice (e.g., prepared as described in Example 2) receiving a composition as described herein comprising a polymeric biomaterial and an A2A and/or A2B adenosine receptor inhibitor (e.g., theophylline) at a tumor resection site survive over a longer period of time (e.g., by at least 10%, 20%, 30%, 40%, 50%, or more), as compared to the control tumor resection mice receiving a control reference composition without an A2A and/or A2B receptor inhibitor. In addition, the group of tumor resection mice receiving said composition comprising a polymeric biomaterial and an A2A and/or A2B receptor inhibitor (e.g., theophylline) exhibit a higher survival rate as compared to the control tumor resection mice receiving a control reference composition without an A2A and/or A2B receptor inhibitor.

In certain embodiments, a group of tumor resection mice (e.g., prepared as described in Example 2) receiving a composition as described herein comprising a polymeric biomaterial and a A2A inhibitor (e.g., istradefylline, AZD4635, MK-3814, and/or any combination thereof) at a tumor resection site survive over a longer period of time (e.g., by at least 10%, 20%, 30%, 40%, 50%, or more), as compared to the control tumor resection mice receiving a control reference composition without a A2A inhibitor. In addition, the group of tumor resection mice receiving said composition comprising a polymeric biomaterial and an A2A inhibitor (e.g., istradefylline, AZD4635, MK-3814, and/or any combination thereof) exhibit a higher survival rate as compared to the control tumor resection mice receiving a control reference composition without an A2A inhibitor.

In certain embodiments, a group of tumor resection mice (e.g., prepared as described in Example 2) receiving a composition as described herein comprising a polymeric biomaterial and a A2B inhibitor (e.g., alloxazine) at a tumor resection site survive over a longer period of time (e.g., by at least 10%, 20%, 30%, 40%, 50%, or more), as compared to the control tumor resection mice receiving a control reference composition without an A2B inhibitor. In addition, the group of tumor resection mice receiving said composition comprising a polymeric biomaterial and an A2B inhibitor (e.g., alloxazine) exhibit a higher survival rate as compared to the control tumor resection mice receiving a control reference composition without an A2B inhibitor.

In certain embodiments, a group of tumor resection mice (e.g., prepared as described in Example 2) receiving a composition as described herein comprising a polymeric biomaterial and a CD73 inhibitor (e.g., AB680, BMS-986179, MEDI9447, and/or any combination thereof) at a tumor resection site survive over a longer period of time (e.g., by at least 10%, 20%, 30%, 40%, 50%, or more), as compared to the control tumor resection mice receiving a control reference composition without a CD73 inhibitor. In addition, the group of tumor resection mice receiving said composition comprising a polymeric biomaterial and a CD73 inhibitor (e.g., AB680, BMS-986179, MEDI9447, and/or any combination thereof) exhibit a higher survival rate as compared to the control tumor resection mice receiving a control reference composition without a CD73 inhibitor. In certain embodiments, a group of tumor resection mice (e.g., prepared as described in Example 2) receiving a composition as described herein comprising a polymeric biomaterial and a P2RX7 signaling inhibitor (e.g., GSK1482160, JNJ-5417544, JNJ-479655, and/or any combination thereof) at a tumor resection site survive over a longer period of time (e.g., by at least 10%, 20%, 30%, 40%, 50%, or more), as compared to the control tumor resection mice receiving a control reference composition without a P2RX7 signaling inhibitor. In addition, the group of tumor resection mice receiving said composition comprising a polymeric biomaterial and a P2RX7 signaling inhibitor (e.g., GSK1482160, JNJ-5417544, JNJ-479655, and/or any combination thereof) exhibit a higher survival rate as compared to the control tumor resection mice receiving a control reference composition without a P2RX7 signaling inhibitor.

### Example 20. Preparation and Uses of Exemplary Composition Described Herein Comprising an ADAR1 Inhibitor

In certain embodiments, a group of tumor resection mice (e.g., prepared as described in Example 2) receiving a composition as described herein comprising a polymeric biomaterial and an ADAR1 inhibitor (e.g., 8-azaadenosine) at a tumor resection site survive over a longer period of time (e.g., by at least 10%, 20%, 30%, 40%, 50%, or more), as compared to the control tumor resection mice receiving a control reference composition without an ADAR1 inhibitor. In addition, the group of tumor resection mice receiving said composition comprising a polymeric biomaterial and an ADAR1 inhibitor (e.g., 8-azaadenosine) exhibit a higher survival rate as compared to the control tumor resection mice receiving a control reference composition without an ADAR1 signaling inhibitor.

### Example 21. Preparation and Uses of Exemplary Composition Described Herein Comprising an Angiotensin H Receptor Antagonist

In certain embodiments, a group of tumor resection mice (e.g., prepared as described in Example 2) receiving a composition as described herein comprising a polymeric biomaterial and an angiotensin II receptor antagonist (e.g., Valsartan) at a tumor resection site survive over a longer period of time (e.g., by at least 10%, 20%, 30%, 40%, 50%, or more), as compared to the control tumor resection mice receiving a control reference composition without an angiotensin II receptor antagonist. In addition, the group of tumor resection mice receiving said composition comprising a polymeric biomaterial and an angiotensin II receptor antagonist (e.g., Valsartan) exhibit a higher survival rate as compared to the control tumor resection mice receiving a control reference composition without an angiotensin II receptor antagonist.

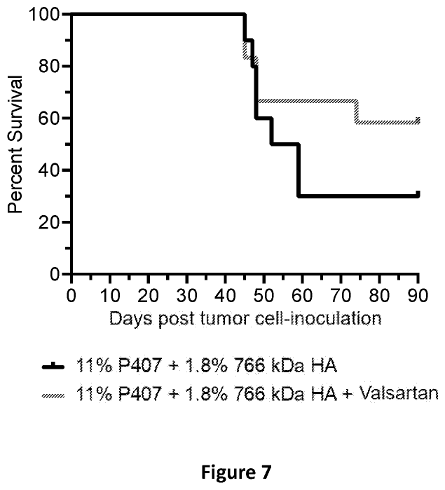

As shown in FIG. 7, the group of tumor resection mice receiving a composition of a polymeric biomaterial of 11% w/w poloxamer 407 and 1.8% w/w 766 kDa HA with an angiotensin II receptor antagonist (e.g., Valsartan, for example, in some embodiments at a dose of 1 mg/mouse) at a tumor resection site survived over a longer period of time as compared to the control group receiving a composition of 11% w/w poloxamer 407 and 1.8% 766 kDa HA without angiotensin II receptor antagonist.

### Example 22. Preparation and Uses of Exemplary Composition Described Herein Comprising a Dopaminergic Receptor Inhibitor and/or an Antipsychotic Agent

In certain embodiments, a group of tumor resection mice (e.g., prepared as described in Example 2) receiving a composition as described herein comprising a polymeric biomaterial and a dopaminergic receptor inhibitor and/or an antipsychotic agent (e.g., Prochlorperazine) at a tumor resection site survive over a longer period of time (e.g., by at least 10%, 20%, 30%, 40%, 50%, or more), as compared to the control tumor resection mice receiving a control reference composition without a dopaminergic receptor inhibitor and/or an antipsychotic agent. In addition, the group of tumor resection mice receiving said composition comprising a polymeric biomaterial and a dopaminergic receptor inhibitor and/or an antipsychotic agent (e.g., Prochlorperazine) exhibit a higher survival rate as compared to the control tumor resection mice receiving a control reference composition without a dopaminergic receptor inhibitor and/or an antipsychotic agent.

### Example 23. Preparation and Uses of Exemplary Composition Described Herein Comprising a TAM Family Receptor Tyrosine Kinase Signaling Pathway Inhibitor

In certain embodiments, a group of tumor resection mice (e.g., prepared as described in Example 2) receiving a composition as described herein comprising a polymeric biomaterial and a TAM family receptor tyrosine kinase signaling pathway inhibitor (e.g., Cabozantinib, Merestinib, BMS-77607, S49076, ONO-7476, RXDX-106, LDC1267, Sitravatinib, UNC2025, and/or any combination thereof) at a tumor resection site survive over a longer period of time (e.g., by at least 10%, 20%, 30%, 40%, 50%, or more), as compared to the control tumor resection mice receiving a control reference composition without a TAM family receptor tyrosine kinase signaling pathway inhibitor. In addition, the group of tumor resection mice receiving said composition comprising a polymeric biomaterial and a TAM family receptor tyrosine kinase signaling pathway inhibitor (e.g., Cabozantinib, Merestinib, BMS-77607, S49076, ONO-7476, RXDX-106, LDC1267, Sitravatinib, UNC2025, and/or any combination thereof) exhibit a higher survival rate as compared to the control tumor resection mice receiving a control reference composition without a TAM family receptor tyrosine kinase signaling pathway inhibitor.

### Example 24. Preparation and Uses of Exemplary Composition Described Herein Comprising an IL-4R Signaling Inhibitor

In certain embodiments, a group of tumor resection mice (e.g., prepared as described in Example 2) receiving a composition as described herein comprising a polymeric biomaterial and a interleukin-4 receptor (IL-4R) signaling inhibitor (e.g., vorinostat) at a tumor resection site survive over a longer period of time (e.g., by at least 10%, 20%, 30%, 40%, 50%, or more), as compared to the control tumor resection mice receiving a control reference composition without a IL-4R signaling inhibitor. In addition, the group of tumor resection mice receiving said composition comprising a polymeric biomaterial and an IL-4R signaling inhibitor (e.g., vorinostat) exhibit a higher survival rate as compared to the control tumor resection mice receiving a control reference composition without an IL-4R signaling inhibitor.

### Example 25. Preparation and Uses of Exemplary Composition Described Herein Comprising a Corticosteroid

In certain embodiments, a group of tumor resection mice (e.g., prepared as described in Example 2) receiving a composition as described herein comprising a polymeric biomaterial and a corticosteroid (e.g., dexamethasone) at a tumor resection site survive over a longer period of time (e.g., by at least 10%, 20%, 30%, 40%, 50%, or more), as compared to the control tumor resection mice receiving a control reference composition without a corticosteroid. In addition, the group of tumor resection mice receiving said composition comprising a polymeric biomaterial and a corticosteroid (e.g., dexamethasone) exhibit a higher survival rate as compared to the control tumor resection mice receiving a control reference composition without a corticosteroid.

### Example 26. Preparation and Uses of Exemplary Composition Described Herein Comprising a Glutamate-Gated Chloride Channel Activator and/or a P2RX4, P2RX7, and/or α7 nAChR Positive Allosteric Effector

In certain embodiments, a group of tumor resection mice (e.g., prepared as described in Example 2) receiving a composition as described herein comprising a polymeric biomaterial and a glutamate-gated chloride channel activator and/or a purinergic receptor P2X4 (P2RX4), purinergic receptor P2X7 (P2RX7), and/or alpha7 nicotinic acetylcholine receptor (α7 nAChR) positive allosteric effector (e.g., ivermectin) at a tumor resection site survive over a longer period of time (e.g., by at least 10%, 20%, 30%, 40%, 50%, or more), as compared to the control tumor resection mice receiving a control reference composition without a glutamate-gated chloride channel activator and/or P2RX4, P2RX7, and/or α7 nAChR positive allosteric effector. In addition, the group of tumor resection mice receiving said composition comprising a polymeric biomaterial and a glutamate-gated chloride channel activator and/or P2RX4, P2RX7, and/or α7 nAChR positive allosteric effector (e.g., ivermectin) exhibit a higher survival rate as compared to the control tumor resection mice receiving a control reference composition without a glutamate-gated chloride channel activator and/or P2RX4, P2RX7, and/or α7 nAChR positive allosteric effector.

### Example 27. Preparation and Uses of Exemplary Composition Described Herein Comprising a Beta-Adrenergic Receptor Antagonist

In certain embodiments, a group of tumor resection mice (e.g., prepared as described in Example 2) receiving a composition as described herein comprising a polymeric biomaterial and a beta-adrenergic receptor antagonist (e.g., propranolol and/or timolol) at a tumor resection site survive over a longer period of time (e.g., by at least 10%, 20%, 30%, 40%, 50%, or more), as compared to the control tumor resection mice receiving a control reference composition without a beta-adrenergic receptor antagonist. In addition, the group of tumor resection mice receiving said composition comprising a polymeric biomaterial and a beta-adrenergic receptor antagonist (e.g., propranolol) exhibit a higher survival rate as compared to the control tumor resection mice receiving a control reference composition without a beta-adrenergic receptor antagonist.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

1. A method comprising a step of:
intraoperative administration at a tumor resection site of a subject suffering from cancer:
a combination of a biomaterial preparation and a modulator of myeloid-derived suppressive cell function.

2. The method of claim 1, wherein the modulator of myeloid-derived suppressive cell function is or comprises a modulator of neutrophil function.

3. The method of claim 2, wherein the modulator of neutrophil function is or comprises an agent that (i) inhibits neutrophil recruitment, (ii) inhibits neutrophil survival and/or proliferation, and/or (iii) modulates neutrophil-associated effector function.

4. The method of claim 3, wherein the agent that modulates neutrophil-associated effector function is characterized by its ability to: (i) modulate production and/or secretion of one or more immunomodulatory cytokines and/or chemokines, and/or (ii) inhibit modification of extracellular matrix by neutrophils at the tumor resection site.

5-7. (canceled)

8. The method of claim 2, wherein the modulator of neutrophil function is selected from the group consisting of: cathepsin G inhibitors, elastase inhibitors, CD74 inhibitors, CD47 inhibitors, adenosine pathway (CD39, CD73, A2AR, A2BR) inhibitors, ADAR1 inhibitors, matrix metalloproteinase (MMP) inhibitors, protein arginine deiminases 4 (PAD4) inhibitors, tyrosine kinases inhibitors, inhibitors of apoptosis proteins (IAP) inhibitors, bruton tyrosine kinase (BTK) inhibitors, purinergic receptor P2X 7 (P2RX7) inhibitors, colony stimulating factor 1 receptor (CSF1R) inhibitors, phosphodiesterase-5 (PDE5) inhibitors, activators of specialized pro-resolving mediators (SPMs), TGF TGFβR1R1 inhibitors, CC chemokine inhibitors (e.g., CCR inhibitors, CCL inhibitors), CXC chemokine inhibitors (e.g., CXCR inhibitors, CXCL inhibitors), metformin, TREM-1 and/or TREM-2 inhibitors, interleukin 34 (IL-34) signaling inhibitors, purinergic receptor P2X4 (P2RX4) inhibitors, interleukin 1α (IL-1α) signaling inhibitors, dopaminergic receptor inhibitors and/or antipsychotic agents, neutropenia causing agents, TAM family receptor tyrosine kinase signaling pathway inhibitors, leukocyte-associated immunoglobulin-like receptor 1 (LAIR-1) inhibitors, leukocyte immunoglobulin-like receptor (LILR) associated signaling pathway modulators, c-Kit related signaling pathway inhibitors, MET related signaling pathway inhibitors, interleukin-4 receptor (IL-4R) signaling inhibitors, monoamine oxidase A (MAO-A) inhibitors, complement component C5a and/or C5a receptor inhibitors, corticosteroids, glutamate-gated chloride channel activator and/or P2RX4, P2RX7, and/or alpha7 nicotinic acetylcholine receptor (α7 nAChR) positive allosteric effectors, beta-adrenergic receptor antagonists, renin-angiotensin system inhibitors, angiopoietin signaling modulators, and any combinations thereof.

9. The method of claim 1, wherein the biomaterial preparation ii)comprises one or more polymers, and/or (ii) is temperature-responsive.

10. (canceled)

11. The method of claim 9, wherein the temperature-responsive biomaterial preparation; (i) has a critical gelation temperature (CGT) of 20-39° C., (ii) comprises a poloxamer, and/or (iii) comprises a poloxamer and a second polymer component that (a) is not a poloxamer and/or (b) is or comprises a carbohydrate polymer.

12-16. (canceled)

17. The method of claim 13, wherein the carbohydrate polymer is or comprises (i) hyaluronic acid or (ii) chitosan or a modified chitosan.

18-20. (canceled)

21. The method of claim 1, wherein the combination is administered at or within 2 cm of the tumor resection site.

22. The method of claim 1, wherein the tumor resection site is characterized by absence of gross residual tumor antigen.

23. The method of claim 1, wherein the biomaterial preparation is administered in (i) a polymer network state, or (ii) a precursor state, wherein the precursor state transitions to a polymer network state upon the administration at the tumor resection site.

24. The method of claim 23, wherein the polymer network state is(ii a hydrogel or (ii) a viscous solution or colloid.

25-26. (canceled)

27. The method of claim 1, wherein the administration:
(i) is by implantation or injection; and/or
(ii) is performed concurrently with or subsequent to: (i) laparoscopy, (ii) minimally invasive surgery, and/or (iii) robotic surgery.

28-32. (canceled)

33. The method of claim 1, wherein the biomaterial preparation:
(i) is characterized by a storage modulus of about 100 Pa to about 50,000 Pa;
(ii) is biodegradable in vivo; and/or
(iii) is characterized in that, when tested in vivo by administering the biomaterial preparation at a mammary fat pad of a mouse subject, less than or equal to 10% of the biomaterial preparation remains in vivo 4 months after the administration.

34. The method of claim 1, wherein the step of administration excludes (i) adoptive transfer of T cells to the subject; (ii) administration of a tumor antigen to the subject; and/or (iii) administration of a microparticle to the subject.

35-36. (canceled)

37. The method of claim 1, wherein the combination further comprises an additional immunomodulatory payload.

38. The method of claim 37, wherein the additional immunomodulatory payload is or comprises: a modulator of innate immunity, a modulator of myeloid cell function, a modulator of adaptive immunity, a modulator of inflammation, or a combination thereof.

39-43. (canceled)

44. The method of claim 1, wherein when the combination is characterized in that a test animal group with spontaneous metastases having, at a tumor resection site, the combination, has a higher percent survival than a comparable test animal group having, at a tumor resection site, a biomaterial preparation without the modulator of myeloid-derived suppressive cell function, as assessed at 2 months or 3 months after the administration.

45. The method of claim 24, wherein the polymer network state of the biomaterial preparation is characterized in that:
(a) when tested in vitro by placing the combination in PBS (pH 7.4), less than 100% of the modulator of myeloid-derived suppressive cell function is released within 3 hours from the biomaterial preparation;
(b) when tested in vitro by placing the combination in PBS (pH 7.4), at least 10% of the modulator of myeloid-derived suppressive cell function is released within 12 hours from the biomaterial preparation, or at least 40% of the modulator of myeloid-derived suppressive cell function is released within 48 hours from the biomaterial preparation;
(c) when tested in vivo by administering the combination at a mammary fat pad of a mouse subject, less than or equal to 50% of the modulator of myeloid-derived suppressive cell function is released in vivo 8 hours after the administration;
(d) it extends release of the modulator of myeloid-derived suppressive cell function so that, when assessed at 24 hours after administration, more modulator of myeloid-derived suppressive cell function is present in the tumor resection site than is observed when the modulator of myeloid-derived suppressive cell function is administered in solution; or
(e) a combination thereof.

46-48. (canceled)

49. A method comprising steps of:
resecting a tumor in a subject suffering from cancer; and
administering at the tumor resection site an extended-release, monotherapeutic polymeric biomaterial formulation of a modulator of myeloid-derived suppressive cell function,
wherein the step of administration excludes (i) adoptive transfer of T cells to the subject; (ii) administration of a tumor antigen to the subject; and (iii) administration of a microparticle to the subject.
